(19) United States
(12) Patent Application Publication
(10) Pub. No.: US 2024/0190857 A1
(43) Pub. Date: Jun. 13, 2024

(54) METHODS FOR MANUFACTURE OF SMALL MOLECULE ACTIVATORS OF TIE-2

(71) Applicant: EyePoint Pharmaceuticals, Inc., Watertown, MA (US)

(72) Inventors: John JANUSZ, Watertown, MA (US); Jared RANDALL, Watertown, MA (US); James COPP, Watertown, MA (US)

(21) Appl. No.: 18/257,712

(22) PCT Filed: Dec. 17, 2021
(86) PCT No.: PCT/US2021/064063

§ 371 Date: Jan. 4, 2024

Related U.S. Application Data
(60) Provisional application No. 63/127,411, filed on Dec. 18, 2020.

(51) Int. Cl. C07D 417/14; C07D 417/04

(52) U.S. Cl. CPC C07D 417/14; C07D 417/04

(57) ABSTRACT

Disclosed herein are compounds effective for modulation of Tie-2 activity and inhibition of HPTP-beta, and methods of preparation thereof. The compounds can provide effective therapy for vascular disorders that can include, for example, retinopathies, ocular edema, and ocular neovascularization.

### CROSS-REFERENCE

This application claims the benefit of U.S. Provisional Application No. 63/127,411, filed Dec. 18, 2020, which is incorporated by reference herein in its entirety.

### BACKGROUND

The human vascular system is an organ system responsible for the delivery of nutrients and removal of waste products from tissues, and the maintenance of homeostasis throughout the body. Tie-2 is a transmembrane tyrosine-protein kinase receptor expressed in the vascular endothelium that regulates vascular stability. Disease states associated with the deactivation of Tie-2 include vascular inflammation and leakage, pathologic neovascularization, and angiogenesis.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the invention provides a pharmaceutical composition comprising a mixture of a Tie-2 modulator and a second compound, wherein: (a) each of the Tie-2 modulator and the second compound has a core structure and a nitrogen atom substituent bound to the core structure at a position on the core structure; (b) the core structure of the Tie-2 modulator is identical to the core structure of the second compound; (c) the position on the core structure of the Tie-2 modulator to which the nitrogen atom substituent is bound is identical to the position on the core structure of the second compound to which the nitrogen atom substituent is bound; (d) the nitrogen atom substituent of the Tie-2 modulator is —N(H)(E), wherein E is a group that contains a sulfur atom bound to the nitrogen atom; (e) the nitrogen atom substituent of the second compound is —NH₂; and (f) the pharmaceutical composition is substantially free of solvent.

In some embodiments, the invention provides a process for preparing a composition, the process comprising: (i) contacting an initial quantity of an amine with a sulfur trioxide source in a solvent to afford a first mixture, wherein the first mixture comprises a quantity of a first ion pair that is a sulfamate anion and an organic cation; and (ii) contacting the first ion pair with a sodium cation source to provide a second mixture, wherein the second mixture comprises a second ion pair and the amine, wherein the second ion pair is a sodium cation and the sulfamate anion, wherein the initial quantity of the amine is at least 1 kg, and a ratio of the sulfamate anion to the amine in the second mixture is at least 99:1 (a/a) as determined by a liquid chromatography assay.

In some embodiments, the invention provides a process comprising reducing a nitro compound in presence of a solvent to provide a reaction mixture comprising an amino compound, wherein the amino compound is a desulfonylation congener of a Tie-2 modulator, and a solubility of the solvent in water is less than about 20 grams of the solvent per 100 grams of water at 20° C.

In some embodiments, the invention provides a process comprising contacting an acid of formula (V):

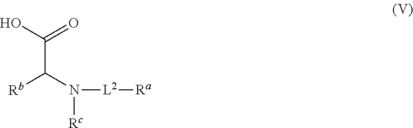

with an amine compound of formula (VI):

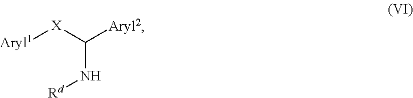

or a salt thereof,
in presence of an amide coupling reagent and a solvent to provide a reaction mixture, the reaction mixture comprising an amide of formula (VII):

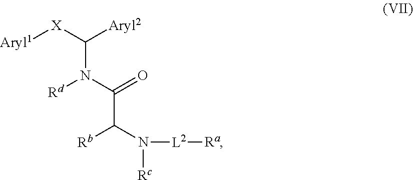

wherein
Aryl¹ is an aryl group which is substituted or unsubstituted;
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond;
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,
wherein a solubility of the solvent in water is less than about 20 grams of the solvent per 100 grams of water at 20° C.

In some embodiments, the invention provides a process comprising contacting a quantity of L-phenylalanine with a quantity of methyl chloroformate in presence of a base and a solvent to form a reaction mixture, wherein the reaction mixture comprises a quantity of a compound of formula (Va1):

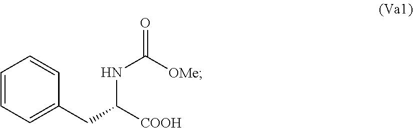 [SMILES: COC(=O)N[C@@H](Cc1ccccc1)C(=O)O]

and a quantity of a side-product of formula (VIII):

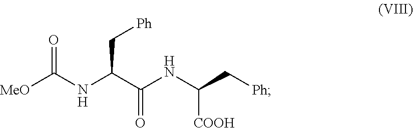 [SMILES: COC(=O)N[C@@H](Cc1ccccc1)C(=O)N[C@@H](Cc1ccccc1)C(=O)O]

and
wherein an area/area ratio of the quantity of the compound of formula (Va1) to the quantity of the side-product of formula (VIII) is at least about 95:5 as determined by a liquid chromatography assay, wherein the assay is performed on a sample of the reaction mixture that is obtained at least about 1 hour after initiation of the contacting, and wherein the quantity of methyl chloroformate is at least 1 kg.

In some embodiments, the invention provides a composition comprising:
a) a compound of formula (Ia6):

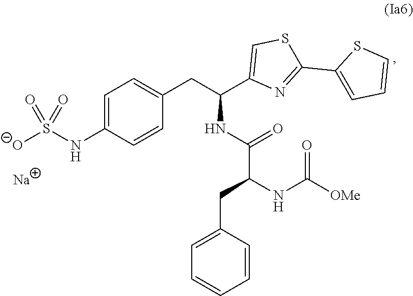 [SMILES: [Na+] ; COC(=O)N[C@@H](Cc1ccccc1)C(=O)N[C@@H](Cc1ccc(NS(=O)(=O)[O-])cc1)c1csc(-c2cccs2)n1]

and
b) a compound of formula (IIa6):

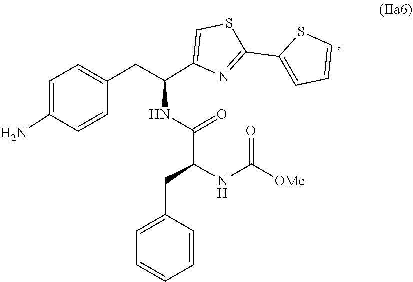 [SMILES: COC(=O)N[C@@H](Cc1ccccc1)C(=O)N[C@@H](Cc1ccc(N)cc1)c1csc(-c2cccs2)n1]

in a mixture, wherein the compound of formula (Ia6) forms at least about 99.0% (a/a) of the composition as determined by UPLC, and wherein the compound of formula (IIa6) forms from about 0.001% to about 0.5% (a/a) of the composition as determined by UPLC, wherein the composition is substantially free of solvent.

In some embodiments, the invention provides a compound of formula (G-2):

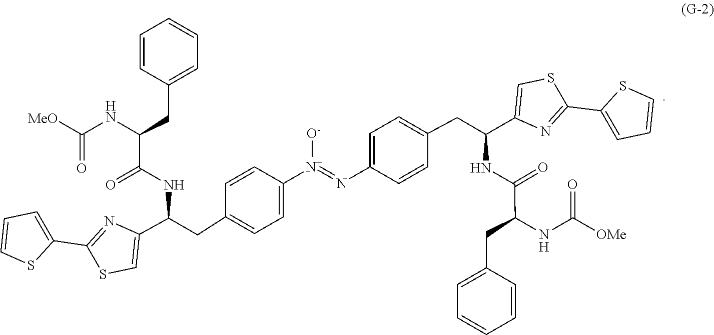 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

### DETAILED DESCRIPTION

Described herein are compounds that can modulate Tie-2 activity, and methods of production thereof. A Tie-2 activator of the disclosure can activate Tie-2 signaling by promoting protein phosphorylation, such as phosphorylation of the Tie-2 protein.

Tie-2 (tyrosine kinase with immunoglobulin and epidermal growth factor homology domains 2) is a membrane receptor tyrosine kinase expressed primarily in vascular endothelial cells and a subset of hematopoietic stem cells (HSCs) and macrophages. The principal regulators of Tie-2 phosphorylation are angiopoietin 1 (Ang-1) and angiopoietin 2 (Ang-2). Ang-1 is an agonist of Tie-2, and binding of Ang-1 to Tie-2 promotes receptor phosphorylation. Ang-2 is a Tie-2 ligand that acts in a context-dependent antagonistic or agonistic manner. Binding of Ang-1 to Tie-2 increases the level of endogenous Tie-2 receptor phosphorylation and initiates downstream AKT signaling. This binding initiates a signaling cascade that can induce distinctive vascular remodeling through highly organized angiogenesis and tightening of the endothelial cell junctions (endothelium cell proximity). Within the vascular endothelium, Ang-1-Tie-2 signaling promotes endothelial cell proximity. In the HSC microenvironment, Ang-1-Tie-2 signaling contributes in a paracrine manner to the long-term repopulation of HSCs.

Under physiological conditions, the duration of Tie-2 phosphorylation is regulated by the human protein tyrosine phosphatase beta (often abbreviated as HPTPβ or HPTP beta), which removes the phosphate from the Tie-2 receptor. By inhibiting HPTPβ, the level of Tie-2 phosphorylation substantially increases, restoring proper cell proximity. HPTPβ plays a functional role in endothelial cell proliferation, viability, differentiation, vasculogenesis, and angiogenesis. HPTPβ and vascular endothelial protein tyrosine phosphatase (VE-PTP; the mouse orthologue of HPTPβ) are expressed in vascular endothelial cells throughout development. A small molecule of the disclosure can activate Tie-2 downstream signaling by inhibiting HPTPβ/VE-PTP.

Compounds that activate Tie-2 can treat disorders and injuries associated with vascular instability, which include, for example, nephropathy, acute kidney injury, cancer, systemic vascular leak syndromes including acute lung injury (ALI) and acute respiratory distress syndrome (ARDS), hypertension including hypertensive crisis/urgency, pulmonary artery hypertension, hepatorenal syndrome, cerebrovascular leakage, and brain edema.

Compounds that activate Tie-2 can treat disorders of the vascular networks of the eye that include, for example, retinopathies, ocular edema, and ocular neovascularization. Non-limiting examples of diseases or conditions that involve retinopathy, ocular edema, or neovascularization can include, for example, diabetic macular edema, age-related macular degeneration (wet form), choroidal neovascularization, diabetic retinopathy, retinal vein occlusion (central or branch), ocular trauma, surgery induced edema, surgery induced neovascularization, cystoid macular edema, ocular ischemia, and uveitis. These diseases or conditions are characterized by changes in the ocular vasculature whether progressive or non-progressive, whether a result of an acute disease or condition, or a chronic disease or condition.

Compounds that activate Tie-2 can also treat disorders related to the impairment of aqueous humor outflow from the anterior chamber of the eye, which can include, for example, glaucoma, primary glaucoma, pseudoexfoliative glaucoma, pigmentary glaucoma, primary juvenile glaucoma, open angle glaucoma, wide-angle glaucoma, close-angle glaucoma, congenital glaucoma, acquired glaucoma, secondary glaucoma, inflammatory glaucoma, phacogenic glaucoma, or neovascular glaucoma. In some embodiments, a Tie-2 activator of the disclosure can stabilize vasculature associated with the trabecular meshwork, thereby reducing intraocular pressure and treating ocular hypertension.

### Activators of Tie-2.

Compounds disclosed herein can be effective as HPTPβ inhibitors and Tie-2 modulators. The compounds can modulate HPTPβ and Tie-2 activity, for example, by binding to or inhibiting HPTPβ. Such compounds can bind to HPTPβ, for example, by mimicking the binding mechanism of a native substrate, such as a phosphorylated compound. A compound can be a phosphate mimetic or bioisostere, for example, a sulfamic acid. The compound could also be derived from an amino acid building block or comprise an amino acid backbone for efficiency and economy of synthesis.

In some embodiments, a compound disclosed herein is a compound of the formula:

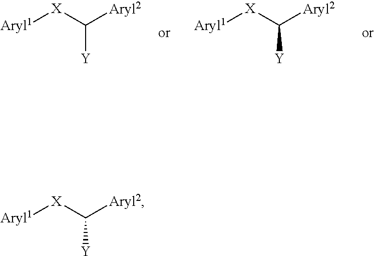

wherein:
Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

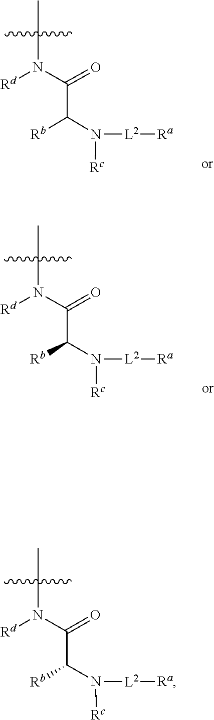

wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted; R^{c} is H or alkyl which is substituted or unsubstituted, or together with any of L², R^{a}, R^{b}, and R^{d} forms a ring that is substituted or unsubstituted; R^{d} is H or alkyl which is substituted or unsubstituted, or together with any of L², R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted, or a pharmaceutically-acceptable salt, tautomer, or zwitterion thereof.

In some embodiments, Aryl¹ is substituted or unsubstituted phenyl, Aryl² is substituted or unsubstituted heteroaryl, and X is alkylene. In some embodiments, Aryl¹ is substituted phenyl, Aryl² is substituted heteroaryl, and X is methylene.

In some embodiments, a compound is of the formula:

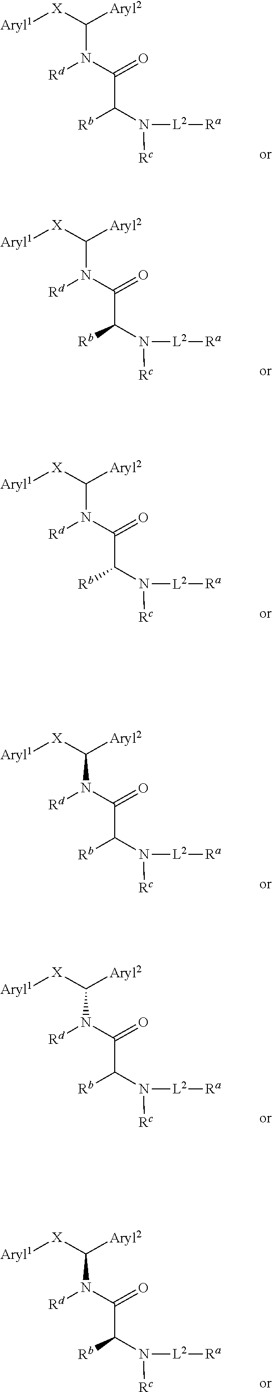

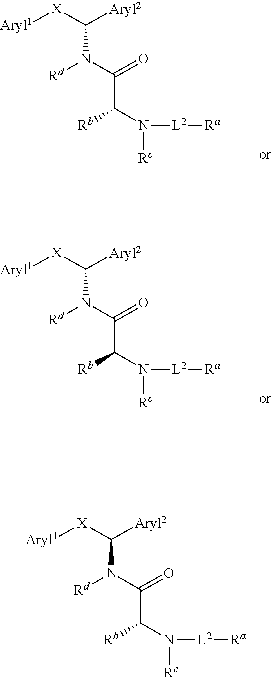

wherein Aryl¹ is para-substituted phenyl, Aryl² is substituted heteroaryl; X is methylene; L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, Aryl¹ is para-substituted phenyl; Aryl² is a substituted thiazole moiety; X is methylene; L² together with the nitrogen atom to which L² is bound forms a carbamate linkage; R^{a} is alkyl, which is substituted or unsubstituted; R^{b} is arylalkyl, which is substituted or unsubstituted; R^{c} is H; and R^{d} is H.

In some embodiments, Aryl² is:

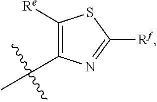

wherein R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, or an alkoxy group, any of which is substituted or unsubstituted and R^{f} is alkyl, aryl, heterocyclyl, or heteroaryl, any of which is substituted or unsubstituted. In some embodiments, Aryl¹ is 4-phenylsulfamic acid; R^{a} is alkyl, which is substituted or unsubstituted; R^{b} is arylalkyl, which is substituted or unsubstituted; R^{e} is H; and R^{f} is heteroaryl. In some embodiments, Aryl¹ is 4-phenylsulfamic acid; R^{a} is alkyl; which is substituted or unsubstituted; R^{b} is arylalkyl, which is substituted or unsubstituted; R^{e} is H; and R^{f} is alkyl.

In some embodiments, Aryl² is:

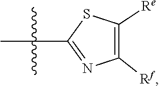

wherein R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted, R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted. In some embodiments, Aryl¹ is 4-phenylsulfamic acid; R^{a} is alkyl, which is substituted or unsubstituted; R^{b} is arylalkyl, which is substituted or unsubstituted; R^{e} is H; and R^{f} is heteroaryl.

In some embodiments, a substituted phenyl group is:

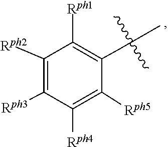

wherein:
each of R^{ph1}, R^{ph2}, R^{ph3}, R^{ph4}, and R^{ph5} is independently H, OH, F, Cl, Br, I, CN, sulfamic acid, tosylate, mesylate, triflate, besylate, alkyl, alkenyl, alkynyl, an alkoxy group, a sulfhydryl group, a nitro group, a nitroso group, an azido group, a sulfoxide group, a sulfone group, a sulfonamide group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl.

Illustrative compounds include the following:

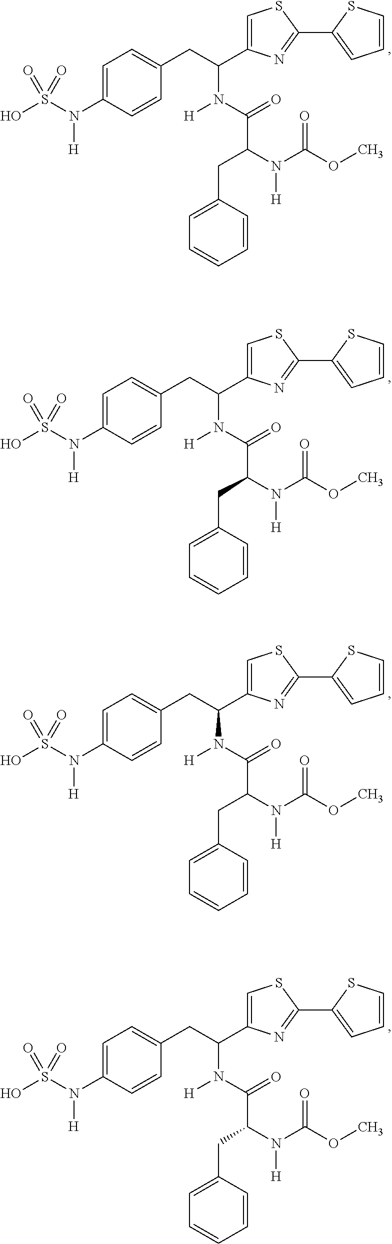 [SMILES: COC(=O)NC(Cc1ccccc1)C(=O)NC(Cc1ccc(NS(=O)(=O)O)cc1)c1csc(-c2cccs2)n1 ; COC(=O)N[C@@H](Cc1ccccc1)C(=O)NC(Cc1ccc(NS(=O)(=O)O)cc1)c1csc(-c2cccs2)n1 ; COC(=O)NC(Cc1ccccc1)C(=O)N[C@@H](Cc1ccc(NS(=O)(=O)O)cc1)c1csc(-c2cccs2)n1 ; COC(=O)N[C@H](Cc1ccccc1)C(=O)NC(Cc1ccc(NS(=O)(=O)O)cc1)c1csc(-c2cccs2)n1]

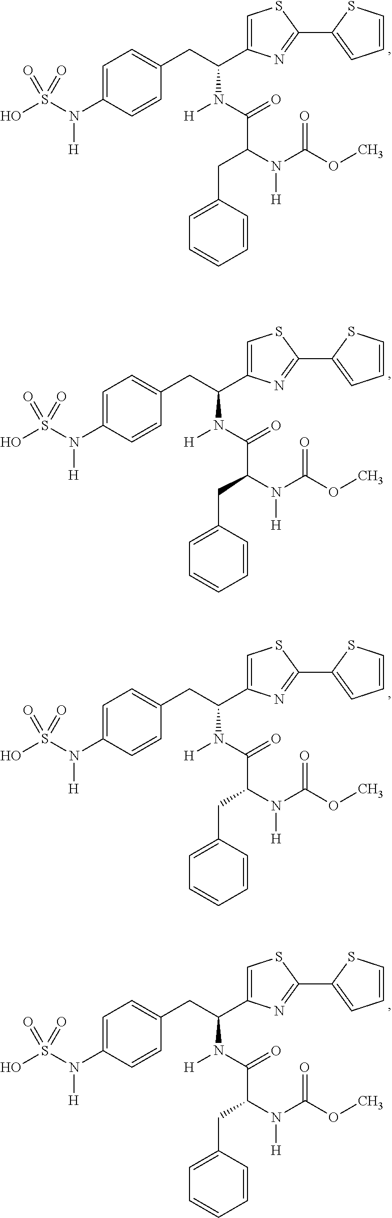 [SMILES: COC(=O)NC(Cc1ccccc1)C(=O)N[C@H](Cc1ccc(NS(=O)(=O)O)cc1)c1csc(-c2cccs2)n1 ; COC(=O)N[C@@H](Cc1ccccc1)C(=O)N[C@@H](Cc1ccc(NS(=O)(=O)O)cc1)c1csc(-c2cccs2)n1 ; COC(=O)N[C@H](Cc1ccccc1)C(=O)N[C@H](Cc1ccc(NS(=O)(=O)O)cc1)c1csc(-c2cccs2)n1 ; COC(=O)N[C@H](Cc1ccccc1)C(=O)N[C@@H](Cc1ccc(NS(=O)(=O)O)cc1)c1csc(-c2cccs2)n1]

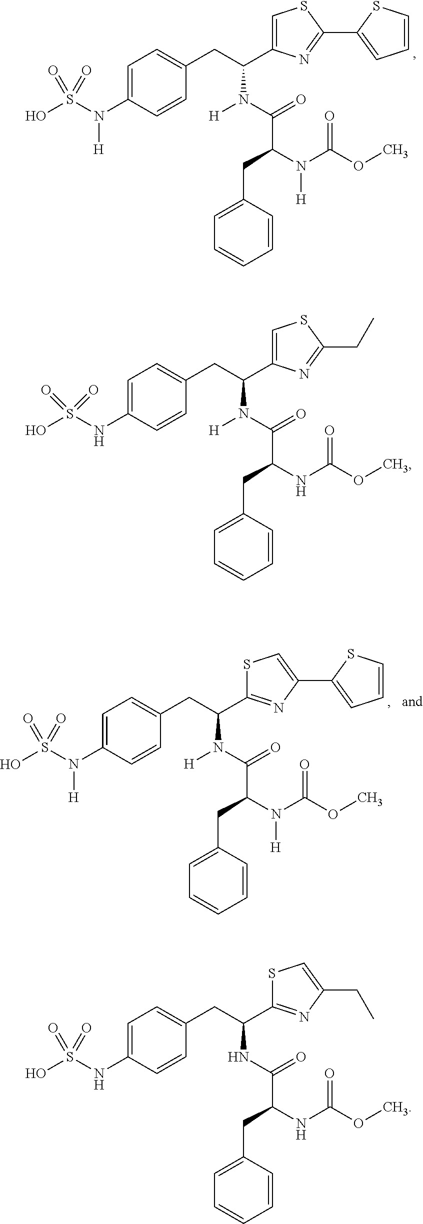 [SMILES: COC(=O)N[C@@H](Cc1ccccc1)C(=O)N[C@H](Cc1ccc(NS(=O)(=O)O)cc1)c1csc(-c2cccs2)n1 ; CCc1nc([C@H](Cc2ccc(NS(=O)(=O)O)cc2)NC(=O)[C@H](Cc2ccccc2)NC(=O)OC)cs1 ; COC(=O)N[C@@H](Cc1ccccc1)C(=O)N[C@@H](Cc1ccc(NS(=O)(=O)O)cc1)c1nc(-c2cccs2)cs1 ; CCc1csc([C@H](Cc2ccc(NS(=O)(=O)O)cc2)NC(=O)[C@H](Cc2ccccc2)NC(=O)OC)n1]

### Optional Substituents for Chemical Groups.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, and ester groups.

Non-limiting examples of alkyl and alkylene groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of alkynyl or alkynylene groups include straight, branched, and cyclic alkynyl groups. The triple bond of an alkynyl or alkynylene group can be internal or terminal. An alkynyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinimide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

### Pharmaceutically-Acceptable Salts.

The present disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrrole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a pyrrole salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

A compound herein can be a salt of an acidic group, for example:

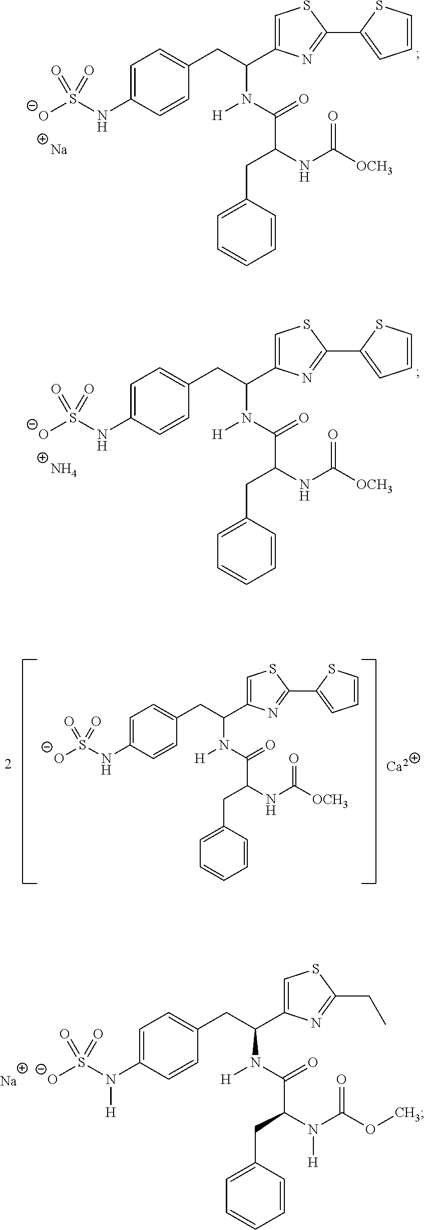

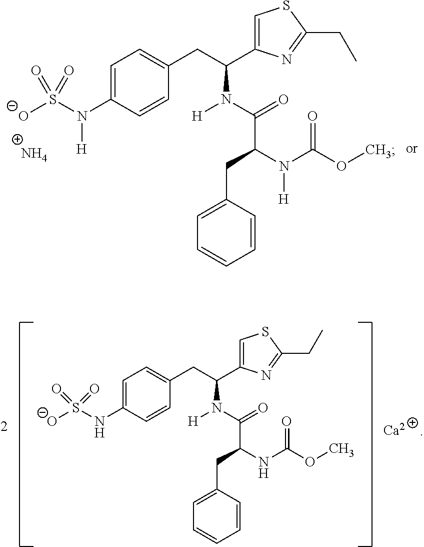

A compound herein can be a salt of a basic group formed from a strong acid, for example:

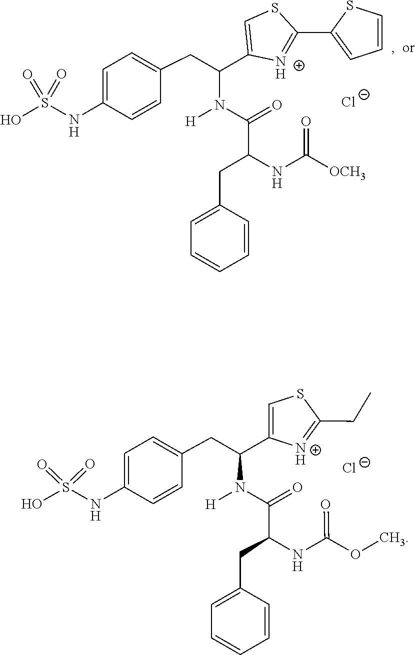 [SMILES: COC(=O)NC(Cc1ccccc1)C(=O)NC(Cc1ccc(NS(=O)(=O)O)cc1)c1csc(-c2cccs2)[nH+]1 ; [Cl-] ; CCc1[nH+]c([C@H](Cc2ccc(NS(=O)(=O)O)cc2)NC(=O)[C@H](Cc2ccccc2)NC(=O)OC)cs1 ; [Cl-]]

A compound herein can also exist in a zwitterionic form, for example:

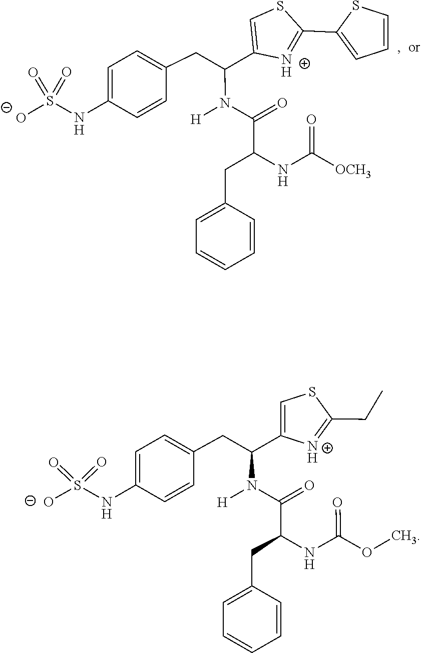 [SMILES: COC(=O)NC(Cc1ccccc1)C(=O)NC(Cc1ccc(NS(=O)(=O)[O-])cc1)c1csc(-c2cccs2)[nH+]1 ; CCc1[nH+]c([C@H](Cc2ccc(NS(=O)(=O)[O-])cc2)NC(=O)[C@H](Cc2ccccc2)NC(=O)OC)cs1]

### Processes for Production of Tie-2 Modulators and Synthetic Intermediates.

### Carbamate Formation.

The present disclosure provides processes for preparation of activators of Tie-2 and pharmaceutically-acceptable salts thereof. The processes disclosed herein include reactions and methods of purification that can produce Tie-2 activators in substantially high yield and purity.

In some embodiments, the processes disclosed herein can provide a synthetic method for acylating phenylalanine with methyl chloroformate to produce intermediate F-2 while avoiding significant formation of acylated dimer G-1 as shown below:

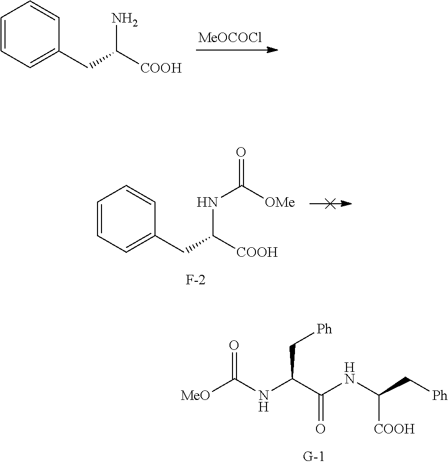

In some embodiments, the reaction is controlled in a manner such that not all of the phenylalanine is converted into another molecular species in the course of the reaction and the formation of dimer G-1 is suppressed. Limited conversion can be achieved by, for example, closely monitoring the process of the reaction via HPLC or another analytical technique and terminating the reaction when the amount of phenylalanine in the reaction mixture is reduced by a certain proportion. Limited conversion can also be achieved by adding a certain amount of methyl chloroformate relative to the amount of phenylalanine such that a limited amount of phenylalanine is converted to another molecular species.

In some embodiments, the disclosure provides a process comprising contacting an initial quantity of L-phenylalanine with an initial quantity of methyl chloroformate in presence of a base and a solvent to form a reaction mixture, wherein the reaction mixture comprises a quantity of a compound of formula (Va1):

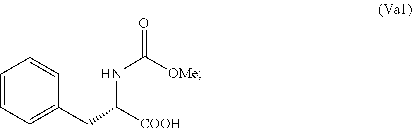 [SMILES: COC(=O)N[C@@H](Cc1ccccc1)C(=O)O]

and a quantity of a side-product of formula (VIII):

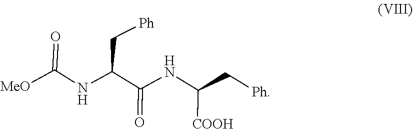 [SMILES: COC(=O)N[C@@H](Cc1ccccc1)C(=O)N[C@@H](Cc1ccccc1)C(=O)O]

In some embodiments, an area/area ratio as determined by a liquid chromatography assay of the quantity of the compound of formula (Va1) to the quantity of the side-product of formula (VIII) is at least about 90:10, at least about 95:5, at least about 99.8:0.2, or at least about 99.9:0.1. In some embodiments, an area/area ratio of the quantity of the compound of formula (Va1) to the quantity of the side-product of formula (VIII) is from about 95:5 to about 99.995:0.005, as determined by an assay. In some embodiments, the assay is performed on a sample of the reaction mixture that is obtained at least about 1 hour after initiation of the contacting. In some embodiments, the assay is performed on a sample of the reaction mixture that is obtained from about 1 hour to about 5 hours after initiation of the contacting. In some embodiments, the assay is a liquid chromatography assay, a gas chromatography assay, a NMR assay, an IR spectroscopy assay, or a Raman spectroscopy assay. In some embodiments, the assay is a HPLC or UPLC assay.

In some embodiments, the initial quantity of methyl chloroformate is at least 0.5 kg. In some embodiments, the initial quantity of methyl chloroformate is at least 1 kg. In some embodiments, the initial quantity of methyl chloroformate is at least 5 kg. In some embodiments, the initial quantity of methyl chloroformate is from about 1 kg to about 100 kg.

In some embodiments, the initial quantity of methyl chloroformate is less than about 1.7 molar equivalents, less than about 1.6 molar equivalents, or less than about 1.5 molar equivalents with respect to the quantity of L-phenylalanine. In some embodiments, the initial quantity of methyl chloroformate is from about 1 to about 1.4 molar equivalents, about 1.1 to about 1.4 molar equivalents, about 1.2 to about 1.4 molar equivalents, about 1.3 to about 1.5 molar equivalents, about 1.3 to about 1.6 molar equivalents, about 1.3 to about 1.7, or about 1.4 to about 1.7 molar equivalents with respect to the quantity of L-phenylalanine. In some embodiments, the initial quantity of methyl chloroformate is from about 1.3 to about 1.4 molar equivalents with respect to the quantity of L-phenylalanine. In some embodiments, the initial quantity of methyl chloroformate is about 1.35 molar equivalents with respect to the quantity of L-phenylalanine.

In some embodiments, an initial quantity of the base is at least about 1.9 molar equivalents, at least about 2 molar equivalents, at least about 2 molar equivalents, at least about 2 molar equivalents, at least about 2.1 molar equivalents, at least about 2.2 molar equivalents, at least about 2.3 molar equivalents, at least about 2.4 molar equivalents, at least about 2.5 molar equivalents, at least about 2.6 molar equivalents, at least about 2.7 molar equivalents, at least about 2.8 molar equivalents, at least about 2.9 molar equivalents, or at least about 3 molar equivalents, with respect to the quantity of L-phenylalanine. In some embodiments, an initial quantity of the base is from about 2.8 to about 3.3 molar equivalents with respect to the quantity of L-phenylalanine. In some embodiments, an initial quantity of the base is from about 2.9 to about 3.1 molar equivalents with respect to the quantity of L-phenylalanine. In some embodiments, an initial quantity of the base is from about 3 to about 3.05 molar equivalents with respect to the quantity of L-phenylalanine. In some embodiments, an initial quantity of the base is about 3.02 molar equivalents with respect to the quantity of L-phenylalanine.

In some embodiments, the contacting comprises: (i) dissolving the quantity of L-phenylalanine and the base in the solvent to provide a basic solution; and (ii) adding the quantity of the methyl chloroformate to the basic solution to form the reaction mixture. In some embodiments, the reaction mixture is maintained at a temperature of less than about 10° C. during (ii). In some embodiments, the reaction mixture is maintained at a temperature from about −20° C. to about 0° C. during (ii). In some embodiments, the adding of (ii) occurs at rate of less than about 2 molar equivalents of methyl chloroformate with respect to the quantity of L-phenylalanine per hour. In some embodiments, the adding of (ii) occurs at rate from about 0.05 to about 1 molar equivalents of methyl chloroformate with respect to the quantity of L-phenylalanine per hour. In some embodiments, the adding of (ii) occurs at rate from about 0.5 to about 0.9 molar equivalents of methyl chloroformate with respect to the quantity of L-phenylalanine per hour. In some embodiments, the adding of (ii) occurs at rate from about 0.6 to about 0.8 molar equivalents of methyl chloroformate with respect to the quantity of L-phenylalanine per hour. In some embodiments, the adding of (ii) occurs at rate of about 0.7 molar equivalents of methyl chloroformate with respect to the quantity of L-phenylalanine per hour.

In some embodiments, the process further comprises adding an organic solvent that is immiscible with water to the reaction mixture when less than about 95%, less than about 96%, or less than about 97% of the quantity of L-phenylalanine has been consumed. In some embodiments, the organic solvent that is immiscible with water is methyl tert-butyl ether (MTBE).

### Synthesis of Compounds of Formula (VII).

In some embodiments, the disclosure provides a process comprising contacting an acid of formula (V):

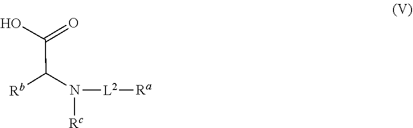

with an amine compound of formula (VI):

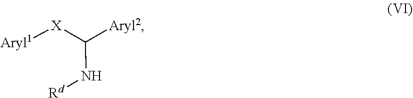

or a salt thereof,
in presence of an amide coupling reagent and a solvent to provide a reaction mixture, wherein the reaction mixture comprises an amide of formula (VII):

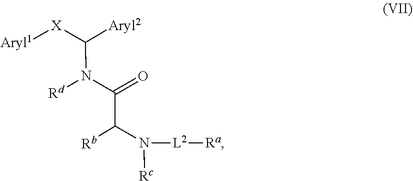

wherein Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted; R^{c} is H or alkyl which is substituted or unsubstituted, or together with any of L², R^{a}, R^{b}, and R^{d} forms a ring that is substituted or unsubstituted; R^{d} is H or alkyl which is substituted or unsubstituted, or together with any of L², R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted, wherein a solubility of the solvent in water is less than about 20 grams of the solvent per 100 grams of water at 20° C.

In some embodiments, Aryl¹ is substituted or unsubstituted phenyl; Aryl² is substituted or unsubstituted heteroaryl; and X is alkylene.

In some embodiments, Aryl¹ is substituted phenyl; Aryl² is substituted heteroaryl; and X is methylene.

In some embodiments,
i) The acid is of formula (Va):

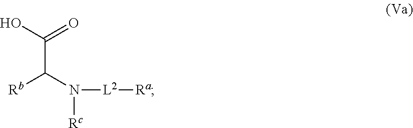

ii) The amine is of formula (VIa):

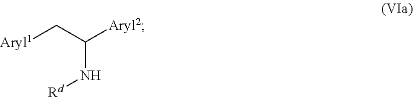

iii) The amide is of formula (VIIa):

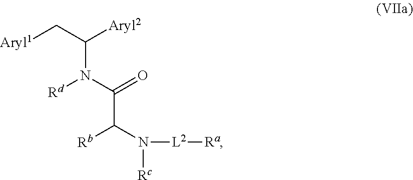

wherein:
Aryl¹ is para-substituted phenyl; Aryl² is substituted heteroaryl; L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, Aryl¹ is para-substituted phenyl; Aryl² is a substituted thiazole moiety; X is methylene; L² together with the nitrogen atom to which L² is bound forms a carbamate linkage; R^{a} is alkyl, which is substituted or unsubstituted; R^{b} is arylalkyl, which is substituted or unsubstituted; R^{c} is H; and R^{d} is H.

In some embodiments, Aryl² is:

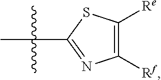

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, Aryl¹ is 4-nitrophenyl; R^{a} is alkyl, which is substituted or unsubstituted; R^{b} is arylalkyl, which is substituted or unsubstituted; R^{e} is H; and R^{f} is heteroaryl.

In some embodiments,
i) The acid is of formula (Va):

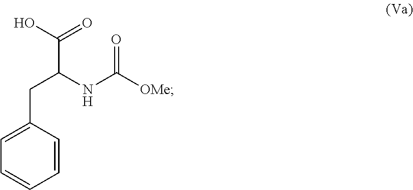 [SMILES: COC(=O)NC(Cc1ccccc1)C(=O)O]

ii) The amine is of formula (VIa):

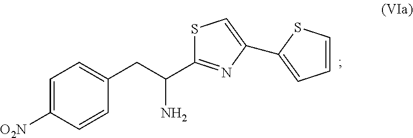 [SMILES: NC(Cc1ccc([N+](=O)[O-])cc1)c1nc(-c2cccs2)cs1]

and
iii) The amide is of formula (VIIa):

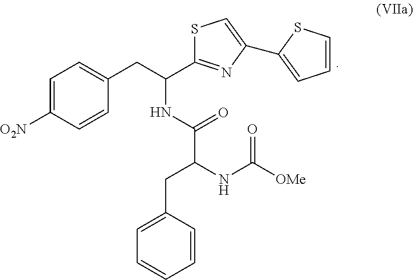 [SMILES: COC(=O)NC(Cc1ccccc1)C(=O)NC(Cc1ccc([N+](=O)[O-])cc1)c1nc(-c2cccs2)cs1]

In some embodiments,
i) The acid is of formula (Va1):

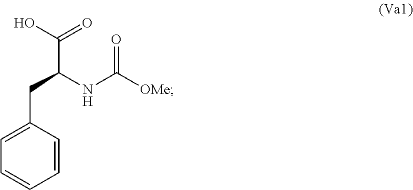 [SMILES: COC(=O)N[C@@H](Cc1ccccc1)C(=O)O]

ii) The amine is of formula (VIa1):

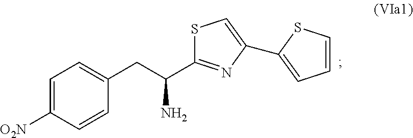 [SMILES: N[C@@H](Cc1ccc([N+](=O)[O-])cc1)c1nc(-c2cccs2)cs1]

and
iii) The amide is of formula (VIIa1):

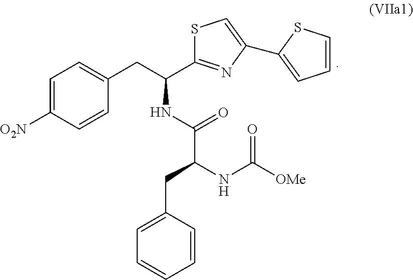 [SMILES: COC(=O)N[C@@H](Cc1ccccc1)C(=O)N[C@@H](Cc1ccc([N+](=O)[O-])cc1)c1nc(-c2cccs2)cs1]

In some embodiments, Aryl² is:

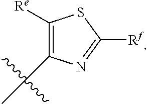

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, Aryl¹ is 4-nitrophenyl; R^{a} is alkyl, which is substituted or unsubstituted; R^{b} is arylalkyl, which is substituted or unsubstituted; R^{e} is H; and R^{f} is alkyl.

In some embodiments,
i) The acid is of formula (Va1):

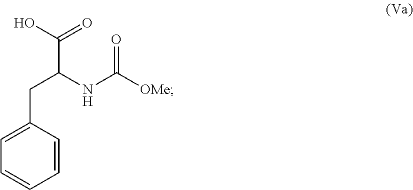 [SMILES: COC(=O)NC(Cc1ccccc1)C(=O)O]

ii) The amine is of formula (VIb):

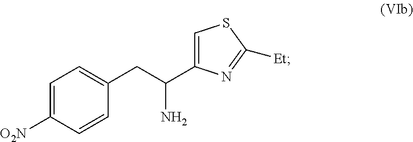 [SMILES: CCc1nc(C(N)Cc2ccc([N+](=O)[O-])cc2)cs1]

and
iii) The amide is of formula (VIIb):

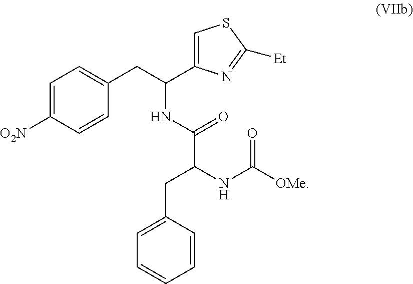 [SMILES: CCc1nc(C(Cc2ccc([N+](=O)[O-])cc2)NC(=O)C(Cc2ccccc2)NC(=O)OC)cs1]

In some embodiments,
i) The acid is of formula (Va1):

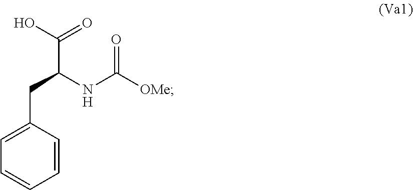 [SMILES: COC(=O)N[C@@H](Cc1ccccc1)C(=O)O]

ii) The amine is of formula (VIb1):

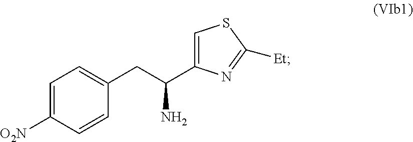 [SMILES: CCc1nc([C@@H](N)Cc2ccc([N+](=O)[O-])cc2)cs1]

and
iii) The amide is of formula (VIIb1):

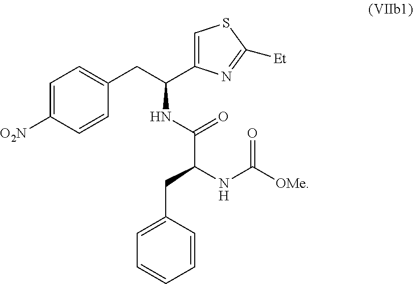 [SMILES: CCc1nc([C@H](Cc2ccc([N+](=O)[O-])cc2)NC(=O)[C@H](Cc2ccccc2)NC(=O)OC)cs1]

In some embodiments, Aryl¹ is 4-nitrophenyl; R^{a} is alkyl, which is substituted or unsubstituted; R^{b} is arylalkyl, which is substituted or unsubstituted; R^{e} is H; and R^{f} is heteroaryl.

In some embodiments,
i) The acid is of formula (Va):

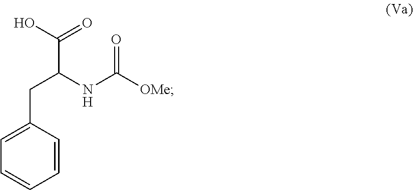 [SMILES: COC(=O)NC(Cc1ccccc1)C(=O)O]

ii) The amine is of formula (VIc):

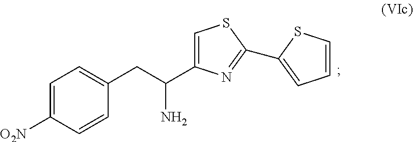 [SMILES: NC(Cc1ccc([N+](=O)[O-])cc1)c1csc(-c2cccs2)n1]

and
iii) The amide is of formula (VIIc):

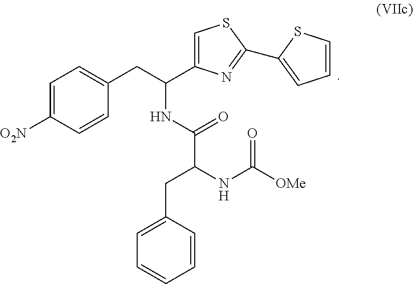 [SMILES: COC(=O)NC(Cc1ccccc1)C(=O)NC(Cc1ccc([N+](=O)[O-])cc1)c1csc(-c2cccs2)n1]

In some embodiments,
i) The acid is of formula (Va1):

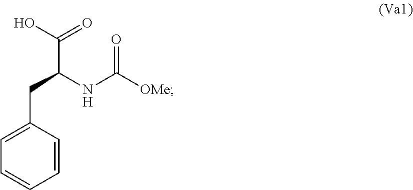 [SMILES: COC(=O)N[C@@H](Cc1ccccc1)C(=O)O]

ii) The amine is of formula (VIc1):

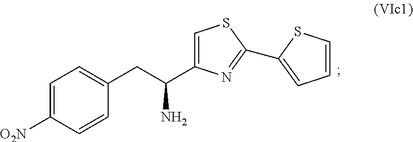 [SMILES: N[C@@H](Cc1ccc([N+](=O)[O-])cc1)c1csc(-c2cccs2)n1]

and
iii) The amide is of formula (VIIc1):

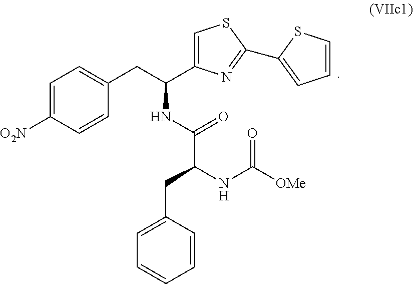 [SMILES: COC(=O)N[C@@H](Cc1ccccc1)C(=O)N[C@@H](Cc1ccc([N+](=O)[O-])cc1)c1csc(-c2cccs2)n1]

In some embodiments, the amine has a solubility of less than about 50 mg/mL in the solvent at a temperature of from about 55° C. to about 60° C. In some embodiments, the amide has a solubility of less than about 30 mg/mL in the solvent at a temperature of from about 60° C. to about 65° C. In some embodiments, the solvent is immiscible in water that has a temperature of 40° C. In some embodiments, the solvent comprises an ether moiety. In some embodiments, the solvent comprises 2-methyltetrahydrofuran. In some embodiments, the base comprises an amine moiety. In some embodiments, the base is N-methylmorpholine.

In some embodiments, the solvent comprises 2-methyltetrahydrofuran, and the reaction mixture comprises a slurry of the amide. In some embodiments, the slurry is further purified by liquid-to-liquid extraction with aqueous washes that are optionally heated to at least about 30° C. In some embodiments, the slurry is diluted with 2-MeTHF prior to liquid-to-liquid extraction. After liquid-liquid extraction, the suspension in 2-MeTHF can be azeodried by iterative distillation of 2-MeTHF/water and redilution with 2-MeTHF. After the azeodrying, the resulting mixture can be diluted with MTBE, filtered, and the resulting retentate can be further washed with MTBE.

In some embodiments, the amide coupling reagent is a carbodiimide, a phosphonium salt, an aminium salt, a uronium salt, or a substituted 1,3,5-triazine. Non-limiting examples of carbodiimide coupling reagents include dicyclohexylcarbodiimide, diisopropylcarbodiimide, and N-(3-dimethylaminopropyl)-N′-ethylcarbodiimide hydrochloride. Non-limiting examples of phosphonium salts include benzotriazol-1-yloxy-tris(dimethylamino)-phosphonium hexafluorophosphate, benzotriazol-1-yloxy-tripyrrolidino-phosphonium hexafluorophosphate, 7-aza-benzotriazol-1-yloxy-tripyrrolidinophosphonium hexafluorophosphate, ethyl cyano(hydroxyimino)acetato-O₂)-tri-(1-pyrrolidinyl)-phosphonium hexafluorophosphate, and 3-(diethoxy-phosphoryloxy)-1,2,3-benzo[d]triazin-4(3H)-one. Non-limiting examples of aminium salts include (2-(1H-benzotriazol-1-yl)-N,N,N′,N′-tetramethylaminium tetrafluoroborate or hexafluorophosphate, 2-(7-aza-1H-benzotriazol-1-yl)-N,N,N′,N′-tetramethylaminium tetrafluoroborate or hexafluorophosphate, and 2-(6-chloro-1H-benzotriazol-1-yl)-N,N,N′,N′-tetramethylaminium hexafluorophosphate. Non-limiting examples of uronium salts include N-[(5-chloro-1H-benzotriazol-1-yl)-dimethylamino-morpholino]-uronium hexafluorophosphate N-oxide, (1-[1-(cyano-2-ethoxy-2-oxoethylideneaminooxy)-dimethylamino-morpholino]-uronium hexafluorophosphate, 2-(1-oxy-pyridin-2-yl)-1,1,3,3-tetramethylisothiouronium tetrafluoroborate, and tetramethylfluoroformamidinium hexafluorophosphate. Non-limiting examples of substituted 1,3,5-triazines include 2-chloro-4,6-dimethoxy-1,3,5-triazine (CDMT), 4-(4,6-dimethoxy-1,3,5-triazin-2-yl)-4-methylmorpholinium halides, 4-(4,6-dimethoxy-1,3,5-triazin-2-yl)-4-methylmorpholinium tetrafluoroborate, polymer-supported 2,4-dichloro-1,3,5-triazine (PS-DCT), and fluorous CDMT. Non-limiting examples of other coupling reagents that can be used include N-ethoxycarbonyl-2-ethoxy-1,2-dihydroquinoline, 2-propanephosphonic acid anhydride, triphosgene, and 1,1′-carbonyldiimidazole.

Each of the aforementioned amide coupling reagents can be used with or without additives that can enhance reactivity and reduce epimerization of substrates and formation of side-products. Such additives can include 1-hydroxybenzotriazole, 1-hydroxybenzotriazole-6-sulfonamidomethyl resin HCl, hydroxy-3,4-dihydro-4-oxo-1,2,3-benzotriazine, N-hydroxysuccinimide, 1-hydroxy-7-aza-1H-benzotriazole, ethyl 2-cyano-2-(hydroximino)acetate, and 4-(N,N-Dimethylamino)pyridine.

The amine of formula (VIc1) can be a free amine base or an ammonium chloride, bromide, or acetate, or another ammonium salt. In some embodiments, the amine is an ammonium bromide salt.

### Reduction.

In some embodiments, the disclosure provides a process comprising reducing a nitro compound in presence of a solvent to provide a reaction mixture comprising an amino compound, wherein the amino compound is a desulfonylation congener of a Tie-2 modulator, and a solubility of the solvent in water is less than about 20 grams of the solvent per 100 grams of water at 20° C.

In some embodiments, the nitro compound comprises a para-nitroarylene moiety.

In some embodiments, the nitro compound is of formula (IV):

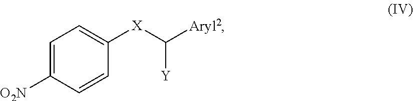

and
the amine is of formula (II):

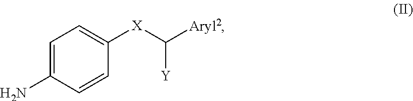

wherein
Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

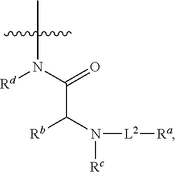

wherein:
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted; R^{c} is H or alkyl which is substituted or unsubstituted, or together with any of L², R^{a}, R^{b}, and R^{d} forms a ring that is substituted or unsubstituted; R^{d} is H or alkyl which is substituted or unsubstituted, or together with any of L², R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted.

In some embodiments, Aryl² is substituted or unsubstituted heteroaryl; and X is alkylene. In some embodiments, Aryl² is substituted heteroaryl; and X is methylene.

In some embodiments,
i) The nitro compound is of formula (IVa):

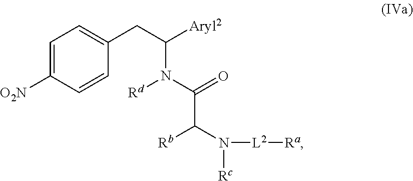

and
ii) The amine is of formula (IIa):

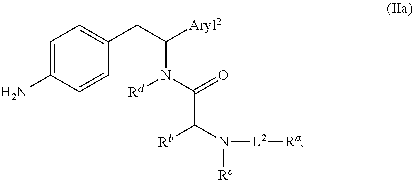

wherein:
Aryl² is substituted heteroaryl; L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, Aryl² is a substituted thiazole moiety; L² together with the nitrogen atom to which L² is bound forms a carbamate linkage; R^{a} is alkyl, which is substituted or unsubstituted; R^{b} is arylalkyl, which is substituted or unsubstituted; R^{c} is H; and R^{d} is H.

In some embodiments, Aryl² is:

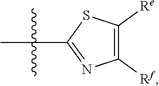

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, R^{a} is alkyl, which is substituted or unsubstituted; R^{b} is arylalkyl, which is substituted or unsubstituted; R^{e} is H; and R^{f} is heteroaryl.

In some embodiments,
i) The nitro compound is of formula (IVa1):

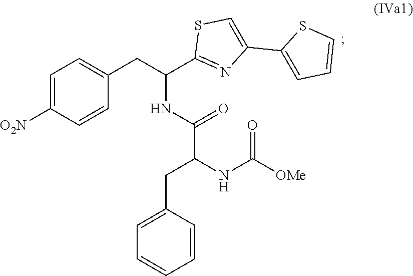 [SMILES: COC(=O)NC(Cc1ccccc1)C(=O)NC(Cc1ccc([N+](=O)[O-])cc1)c1nc(-c2cccs2)cs1]

and
ii) The amine is of formula (IIa1):

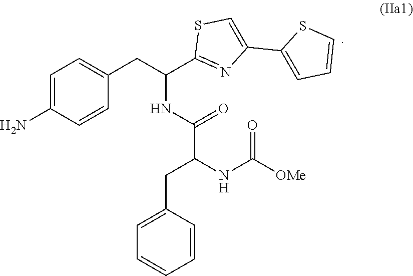 [SMILES: COC(=O)NC(Cc1ccccc1)C(=O)NC(Cc1ccc(N)cc1)c1nc(-c2cccs2)cs1]

In some embodiments,
i) The nitro compound is of formula (Ia2):

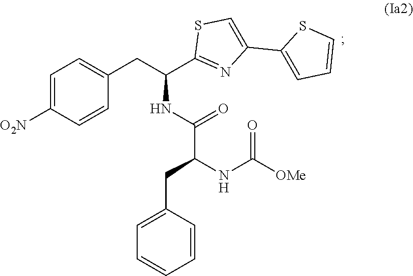 [SMILES: COC(=O)N[C@@H](Cc1ccccc1)C(=O)N[C@@H](Cc1ccc([N+](=O)[O-])cc1)c1nc(-c2cccs2)cs1]

and
ii) The amine is of formula (IIa2):

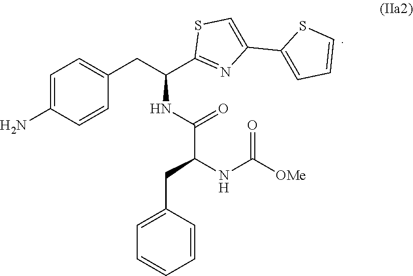 [SMILES: COC(=O)N[C@@H](Cc1ccccc1)C(=O)N[C@@H](Cc1ccc(N)cc1)c1nc(-c2cccs2)cs1]

In some embodiments, Aryl² is:

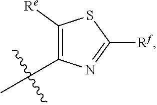

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, R^{a} is alkyl, which is substituted or unsubstituted; R^{b} is arylalkyl, which is substituted or unsubstituted; R^{e} is H; and R^{f} is alkyl.

In some embodiments,
i) The nitro compound is of formula (IVa3):

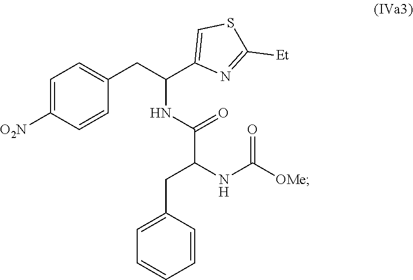 [SMILES: CCc1nc(C(Cc2ccc([N+](=O)[O-])cc2)NC(=O)C(Cc2ccccc2)NC(=O)OC)cs1]

and
ii) The amine is of formula (IIa3):

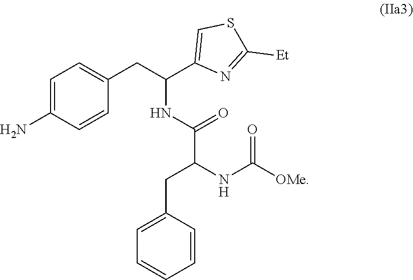 [SMILES: CCc1nc(C(Cc2ccc(N)cc2)NC(=O)C(Cc2ccccc2)NC(=O)OC)cs1]

In some embodiments,
i) The nitro compound is of formula (IVa4):

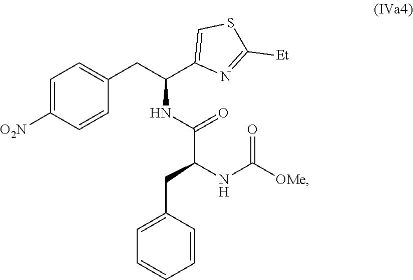 [SMILES: CCc1nc([C@H](Cc2ccc([N+](=O)[O-])cc2)NC(=O)[C@H](Cc2ccccc2)NC(=O)OC)cs1]

and
ii) The amine is of formula (IIa4):

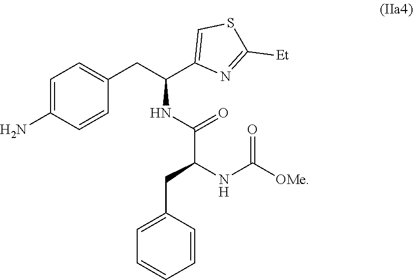 [SMILES: CCc1nc([C@H](Cc2ccc(N)cc2)NC(=O)[C@H](Cc2ccccc2)NC(=O)OC)cs1]

In some embodiments, R^{a} is alkyl, which is substituted or unsubstituted; R^{b} is arylalkyl, which is substituted or unsubstituted; R^{e} is H; and R^{f} is heteroaryl.

In some embodiments,
i) The nitro compound is of formula (IVa5):

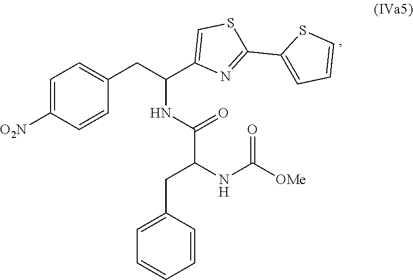 [SMILES: COC(=O)NC(Cc1ccccc1)C(=O)NC(Cc1ccc([N+](=O)[O-])cc1)c1csc(-c2cccs2)n1]

and
ii) The amine is of formula (IIa5):

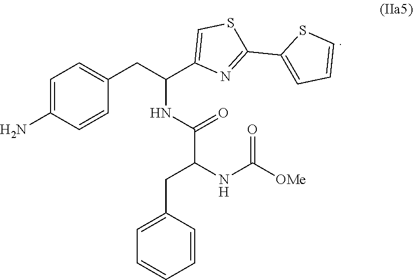 [SMILES: COC(=O)NC(Cc1ccccc1)C(=O)NC(Cc1ccc(N)cc1)c1csc(-c2cccs2)n1]

In some embodiments,
i) The nitro compound is of formula (IVa6):

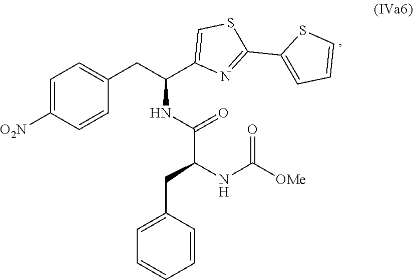 [SMILES: COC(=O)N[C@@H](Cc1ccccc1)C(=O)N[C@@H](Cc1ccc([N+](=O)[O-])cc1)c1csc(-c2cccs2)n1]

and
ii) The amine is of formula (IIa6):

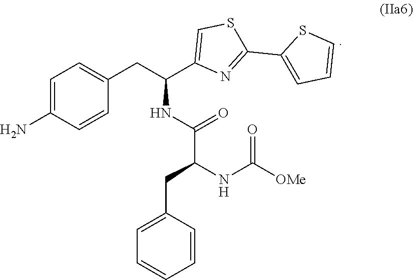 [SMILES: COC(=O)N[C@@H](Cc1ccccc1)C(=O)N[C@@H](Cc1ccc(N)cc1)c1csc(-c2cccs2)n1]

In some embodiments, the solvent comprises 2-methyltetrahydrofuran.

In some embodiments, the reducing comprises contacting the nitro compound with a catalyst in the presence of H₂. In some embodiments, the reducing comprises contacting the nitro compound with a metal-containing catalyst in the presence of H₂. In some embodiments, the reducing comprises contacting the nitro compound with a heterogeneous catalyst in the presence of H₂, wherein the heterogeneous catalyst is palladium on carbon, platinum on carbon, or Raney nickel. In some embodiments, the reducing comprises contacting the nitro compound with a palladium-based catalyst in the presence of H₂. In some embodiments, the reducing comprises contacting the nitro compound with a palladium on carbon in the presence of H₂. In some embodiments, the reducing comprises contacting the nitro compound with palladium on carbon in the presence of H₂ wherein the palladium on carbon comprises from about 3% to about 8% palladium by weight. In some embodiments, the reducing comprises contacting the nitro compound with palladium on carbon in the presence of H₂, wherein the palladium on carbon comprises about 5% palladium by weight. In some embodiments, the reducing is conducted at a temperature of from about 35° C. to about 55° C. In some embodiments, the reducing is conducted at a temperature of about 45° C.

In some embodiments, the catalyst is removed from the reaction mixture by filtration. In some embodiments, the process further comprises precipitating the amine via concentration of the reaction mixture to provide a concentrate, cooling the concentrate to a temperature of about 50° C. to about 60° C., and adding methyl tert-butyl ether (MTBE) to provide a suspension. In some embodiments, the process further comprises stirring the suspension at about 45° C. to about 60° C., and cooling the suspension to about 5° C. to about 15° C. The cooled suspension can be filtered, and the retentate can be washed with MTBE cooled to about 5° C. to about 15° C. The washed retentate can then be dried at about 40° C. to about 50° C. under reduced pressure.

In some embodiments, the reaction mixture further comprises an azoxy compound. In some embodiments, the azoxy compound is a reduction congener of the nitro compound. In some embodiments, the azoxy compound is

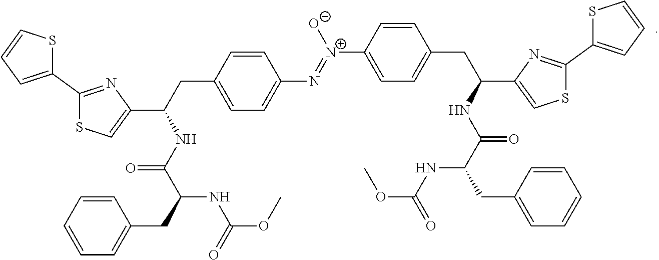 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

### Sulfonylation and Ion Exchange.

In some embodiments, the disclosure provides a process for preparing a composition, the process comprising: (i) contacting an initial quantity of an amine with a sulfur trioxide source in a solvent to afford a first mixture, wherein the first mixture comprises a first ion pair that is a sulfamate anion and an organic cation; and (ii) contacting the first ion pair with a sodium cation source to provide a second mixture, wherein the second mixture comprises a second ion pair and the amine, wherein the second ion pair is a sodium cation and the sulfamate anion, wherein the initial quantity of the amine is at least 1 kg, and a ratio of the sulfamate anion to the amine in the second mixture is at least 99:1 (a/a) as determined by a liquid chromatography assay.

In some embodiments, the initial quantity of amine is contacted with the sulfur trioxide source in the presence of an organic solvent. In some embodiments, the initial quantity of amine is contacted with the sulfur trioxide source in the presence of tetrahydrofuran.

In some embodiments, the initial quantity of amine is contacted with the sulfur trioxide source in the presence of an amine base. In some embodiments, the initial quantity of amine is contacted with the sulfur trioxide source in the presence of triethylamine.

In some embodiments, the process further comprises filtering the second mixture through activated carbon to provide a filtrate. In some embodiments, the process further comprises treating the filtrate to provide a solid, wherein the solid comprises the second ion pair and the amine. In some embodiments, the treating comprises adding an antisolvent to the filtrate to provide a suspension and isolating the solid from the suspension. In some embodiments, the antisolvent is MTBE.

In some embodiments, the process further comprises treating the second mixture to provide a solid, wherein the solid comprises the second ion pair and the amine. In some embodiments, the treating comprises adding an antisolvent to the second mixture to provide a suspension and isolating the solid from the suspension. In some embodiments, the antisolvent is MTBE.

In some embodiments, the amine comprises an arylene moiety substituted with a primary amine.

In some embodiments,
a) the sulfamate anion comprises the moiety:

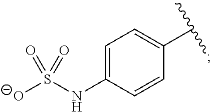

and
b) the amine comprises the moiety:

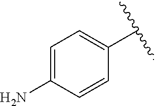

In some embodiments,
a) the sulfamate anion is of formula (I):

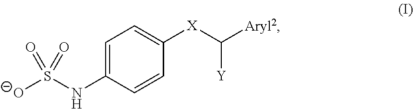

and
b) the amine is of formula (II):

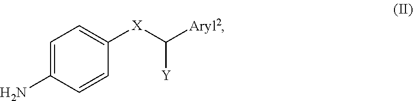

wherein
Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

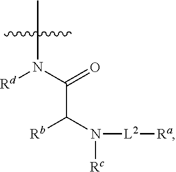

wherein:
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted; R^{c} is H or alkyl which is substituted or unsubstituted, or together with any of L², R^{a}, R^{b}, and R^{d} forms a ring that is substituted or unsubstituted; R^{d} is H or alkyl which is substituted or unsubstituted, or together with any of L², R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted.

In some embodiments, Aryl² is substituted or unsubstituted heteroaryl; and X is alkylene. In some embodiments, Aryl² is substituted heteroaryl; and X is methylene.

In some embodiments,
i) the sulfamate anion is of formula (Ia):

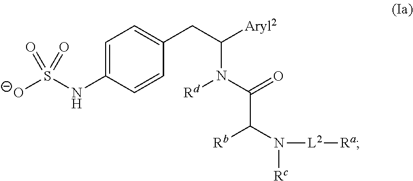

and
ii) the amine is of formula (IIa):

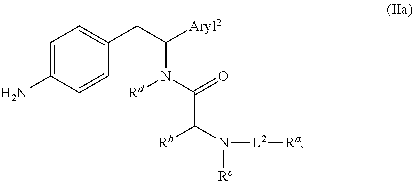

wherein:
Aryl² is substituted heteroaryl; L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, Aryl² is a substituted thiazole moiety; L² together with the nitrogen atom to which L² is bound forms a carbamate linkage; R^{a} is alkyl, which is substituted or unsubstituted; R^{b} is arylalkyl, which is substituted or unsubstituted; R^{c} is H; and R^{d} is H.

In some embodiments, Aryl² is:

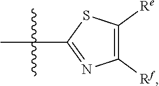

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, R^{a} is alkyl, which is substituted or unsubstituted; R^{b} is arylalkyl, which is substituted or unsubstituted; R^{e} is H; and R^{f} is heteroaryl.

In some embodiments,
i) the sulfamate anion is of formula (Ia1):

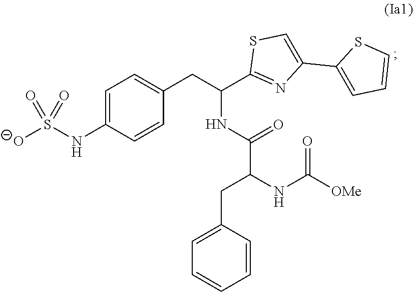 [SMILES: COC(=O)NC(Cc1ccccc1)C(=O)NC(Cc1ccc(NS(=O)(=O)[O-])cc1)c1nc(-c2cccs2)cs1]

and
ii) the amine is of formula (IIa1):

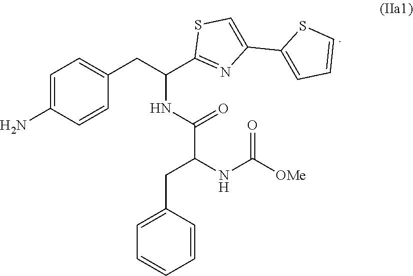 [SMILES: COC(=O)NC(Cc1ccccc1)C(=O)NC(Cc1ccc(N)cc1)c1nc(-c2cccs2)cs1]

In some embodiments,
i) the sulfamate anion is of formula (Ia2):

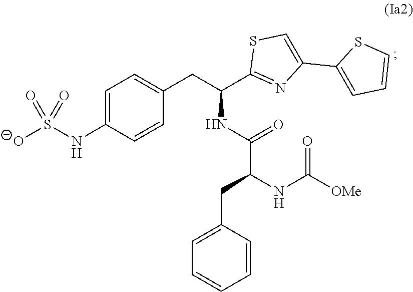 [SMILES: COC(=O)N[C@@H](Cc1ccccc1)C(=O)N[C@@H](Cc1ccc(NS(=O)(=O)[O-])cc1)c1nc(-c2cccs2)cs1]

and
ii) the amine is of formula (IIa2):

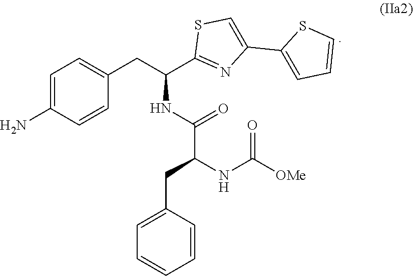 [SMILES: COC(=O)N[C@@H](Cc1ccccc1)C(=O)N[C@@H](Cc1ccc(N)cc1)c1nc(-c2cccs2)cs1]

In some embodiments, Aryl² is:

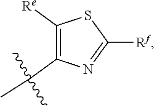

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, R^{a} is alkyl, which is substituted or unsubstituted; R^{b} is arylalkyl, which is substituted or unsubstituted; R^{e} is H; and R^{f} is alkyl.

In some embodiments,
i) the sulfamate anion is of formula (Ia3):

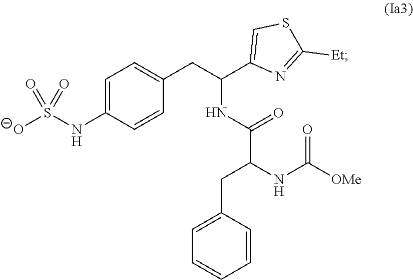 [SMILES: CCc1nc(C(Cc2ccc(NS(=O)(=O)[O-])cc2)NC(=O)C(Cc2ccccc2)NC(=O)OC)cs1]

and
ii) the amine is of formula (IIa3):

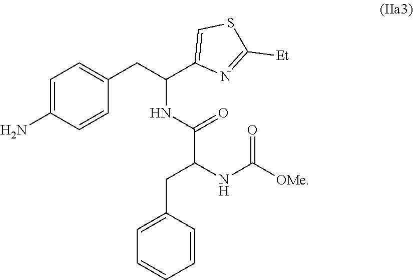 [SMILES: CCc1nc(C(Cc2ccc(N)cc2)NC(=O)C(Cc2ccccc2)NC(=O)OC)cs1]

In some embodiments,
i) the sulfamate anion is of formula (Ia4):

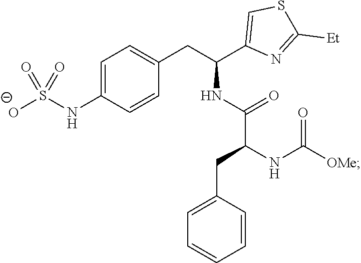 [SMILES: CCc1nc([C@H](Cc2ccc(NS(=O)(=O)[O-])cc2)NC(=O)[C@H](Cc2ccccc2)NC(=O)OC)cs1]

and
ii) the amine is of formula (IIa4):

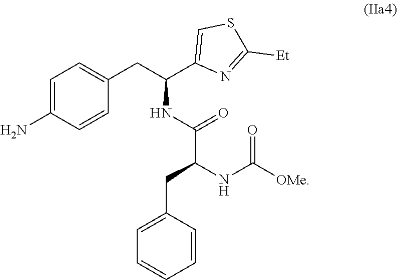 [SMILES: CCc1nc([C@H](Cc2ccc(N)cc2)NC(=O)[C@H](Cc2ccccc2)NC(=O)OC)cs1]

In some embodiments, R^{a} is alkyl, which is substituted or unsubstituted; R^{b} is arylalkyl, which is substituted or unsubstituted; R^{e} is H; and R^{f} is heteroaryl.

In some embodiments,
i) the sulfamate anion is of formula (Ia5):

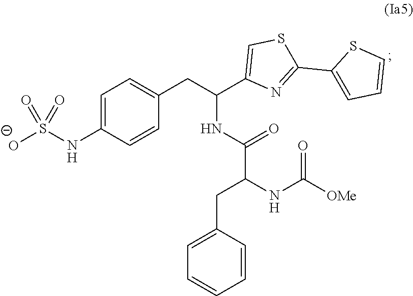 [SMILES: COC(=O)NC(Cc1ccccc1)C(=O)NC(Cc1ccc(NS(=O)(=O)[O-])cc1)c1csc(-c2cccs2)n1]

and
ii) the amine is of formula (IIa5):

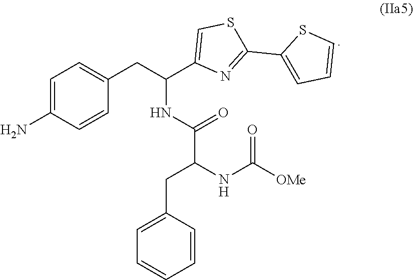 [SMILES: COC(=O)NC(Cc1ccccc1)C(=O)NC(Cc1ccc(N)cc1)c1csc(-c2cccs2)n1]

In some embodiments,
i) the sulfamate anion is of formula (Ia6):

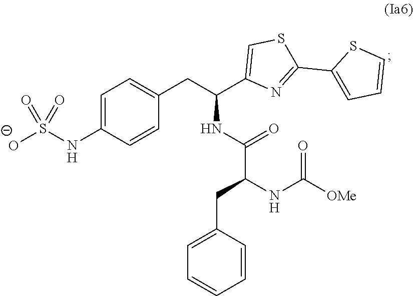 [SMILES: COC(=O)N[C@@H](Cc1ccccc1)C(=O)N[C@@H](Cc1ccc(NS(=O)(=O)[O-])cc1)c1csc(-c2cccs2)n1]

ii) the amine is of formula (IIa6):

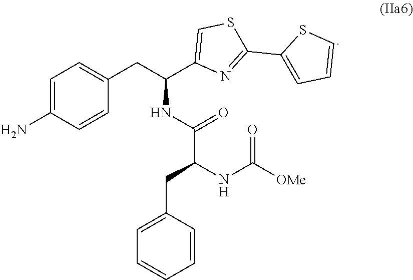 [SMILES: COC(=O)N[C@@H](Cc1ccccc1)C(=O)N[C@@H](Cc1ccc(N)cc1)c1csc(-c2cccs2)n1]

In some embodiments, the organic cation is a trialkylammonium cation. In some embodiments, the organic cation is HNMe₃⁺.

In some embodiments, an area/area ratio of the sulfamate anion to the amine in the second mixture is at least 99.1:0.9, at least 99.2:0.8, or at least 99.3:0.7 as determined by the liquid chromatography assay.

In some embodiments, the second ion pair forms at least about 99.2% (a/a), 99.3% (a/a), or 99.4% (a/a) of the solid as determined by the liquid chromatography assay. In some embodiments, the second ion pair forms from about 99.3% to about 99.5% (a/a) of the solid as determined by the liquid chromatography assay.

In some embodiments, the amine forms about 0.001% to about 0.1% (a/a), from about 0.01% to about 0.1% (a/a), or from about 0.01% to about 0.03% (a/a) of the solid as determined by the liquid chromatography assay.

In some embodiments, the solid comprises no more than about 100 ppm, no more than about 50 ppm, no more than about 10 ppm, no more than about 5 ppm, or no more than about 1 ppm of an azoxy compound. In some embodiments, the solid comprises from about 0.01 ppm to about 100 ppm, from about 0.01 ppm to about 10 ppm, about 0.01 ppm to about 5 ppm, from about 0.01 ppm to about 1 ppm, or from about 0.001 ppm to about 100 ppm of an azoxy compound. In some embodiments, the azoxy compound is

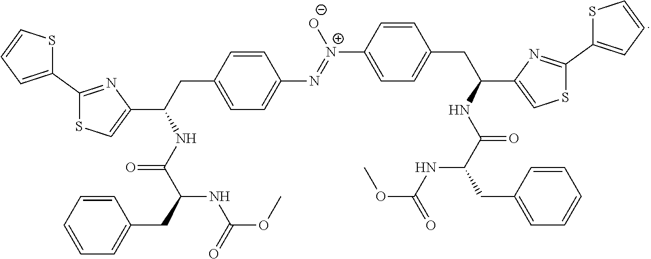 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

In some embodiments, the initial quantity of the amine is at least 10 kg. In some embodiments, the initial quantity of the amine is from about 1 kg to about 100 kg.

In some embodiments, a chemical yield of the second ion pair is at least about 60%, 70%, 80%, or 90% with respect to the initial quantity of the amine. In some embodiments, a chemical yield of the second ion pair is from about 70% to about 90%, from about 70% to about 95%, or from about 70% to about 99% with respect to the initial quantity of the amine.

In some embodiments, a chemical yield of the second ion pair is at least about 1 kg, at least about 2 kg, at least about 3 kg, at least about 4 kg, at least about 5 kg, or at least about 10 kg. In some embodiments, a chemical yield of the second ion pair is from about 10 kg to about 100 kg. In some embodiments, a chemical yield of the second ion pair is from about 10 kg to about 20 kg.

In some embodiments, the sulfur trioxide source is a complex of sulfur trioxide and an organic molecule that comprises a nitrogen atom. In some embodiments, the molecule that comprises the nitrogen atom is trimethylamine, triethylamine, diisopropylethylamine, dimethylaniline, diethylaniline, quinoline, pyridine, tributylamine, N-methylpyrrolidine, dimethylformamide, or dimethylacetamide. In some embodiments, the sulfur trioxide source is a complex of sulfur trioxide and trimethylamine.

In some embodiments, a chemical yield of the second ion pair is at least about 80%, at least about 90%, or at least about 95% with respect to the quantity of the first ion pair. In some embodiments, a chemical yield of the second ion pair is from about 95% to about 99%, with respect to the quantity of the first ion pair.

In some embodiments, the sodium cation source comprises an alkoxide salt, a carbonate salt, a hydroxide salt, an alkoxide salt, a phosphate salt, or a hexamethyldisilazide salt. In some embodiments, the sodium cation source comprises sodium methoxide, sodium ethoxide, or sodium tert-butoxide.

In some embodiments, the process further comprises heating the first mixture to about 25° C. to about 45° C. In some embodiments, the process further comprises filtering the second mixture to provide a filtrate. The filtrate can be added to MTBE to provide a suspension that can be filtered to provide a retentate comprising the second ion pair.

In some embodiments, the contacting the first ion pair with a sodium cation source comprises mixing the first ion pair with about 0.01% to about 0.1% w/w methanolic sodium methoxide to provide a first solution, and contacting the first solution with about 1% to about 4% methanolic sodium methoxide to provide the second mixture.

In some embodiments, the process further comprises filtering the second mixture to provide a filtrate. The filtrate can be further purified by passage of the filtrate through charcoal, such as activated carbon or activated charcoal to provide a treated filtrate. In some embodiments, the treatment of the filtrate with charcoal reduces an azoxy impurity content.

In some embodiments, the treated filtrate is concentrated under reduced pressure and rediluted with MTBE to provide a suspension. The suspension can then be filtered to provide the second ion pair as the retentate.

### Pharmaceutical Compositions.

In some embodiments, the disclosure provides a pharmaceutical composition comprising a mixture of a Tie-2 modulator and a second compound, wherein:
(a) each of the Tie-2 modulator and the second compound has a core structure and a nitrogen atom substituent bound to the core structure at a position on the core structure;
(b) the core structure of the Tie-2 modulator is identical to the core structure of the second compound;
(c) the position on the core structure of the Tie-2 modulator to which the nitrogen atom substituent is bound is identical to the position on the core structure of the second compound to which the nitrogen atom substituent is bound;
(d) the nitrogen atom substituent of the Tie-2 modulator is —N(H)(E), wherein E is a group that contains a sulfur atom bound to the nitrogen atom;
(e) the nitrogen atom substituent of the second compound is —NH₂; and
(f) the pharmaceutical composition is substantially free of solvent.

In some embodiments, the Tie-2 modulator forms at least about 99.0% (a/a) of the mixture as determined by a liquid chromatography assay, and wherein the second compound forms from about 0.001% to about 0.5% (a/a), from about 0.001% to about 0.1% (a/a), from about 0.01% to about 0.1% (a/a), or from about 0.01% to about 0.03% (a/a) of the mixture as determined by the liquid chromatography assay.

In some embodiments, the nitrogen atom substituent of the Tie-2 modulator is a sulfamate group. In some embodiments, the second compound is a desulfonylation congener of the Tie-2 modulator.

In some embodiments, the composition comprises no more than about 100 ppm, no more than about 50 ppm, no more than about 10 ppm, no more than about 5 ppm, or no more than about 1 ppm of a third compound as determined by HPLC, wherein the third compound comprises an azoxy moiety. In some embodiments, the solid comprises from about 0.01 ppm to about 100 ppm, from about 0.01 ppm to about 10 ppm, about 0.01 ppm to about 5 ppm, from about 0.01 ppm to about 1 ppm, or from about 0.001 ppm to about 100 ppm of the third compound. In some embodiments, the composition is substantially free of a third compound as determined by HPLC, wherein the third compound comprises an azoxy moiety.

In some embodiments, the composition comprises a third compound that has a structure of Z-J-Z, wherein J is

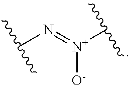 [SMILES: */N=[N+](/*)[O-]]

In some embodiments, the composition comprises no more than about 100 ppm, no more than about 50 ppm, no more than about 10 ppm, no more than about 5 ppm, or no more than about 1 ppm of a third compound as determined by HPLC, wherein the third compound has a structure of Z-J-Z, wherein J is

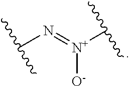 [SMILES: */N=[N+](/*)[O-]]

In some embodiments, the Tie-2 modulator has a structure of Q-Z; and the second compound has a structure of W—Z, wherein

Q is

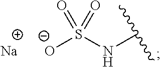 [SMILES: [Na+] ; *NS(=O)(=O)[O-] ; ";"]

W is H₂N—; and
each Z is the core structure.

In some embodiments, each Z is:

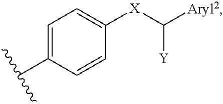

wherein
Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; and Y is H, aryl, heteroaryl, NH(aryl), NH(heteroaryl), or NHCOR^{g}, any of which is substituted or unsubstituted, or

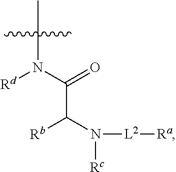

wherein:
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted; R^{c} is H or alkyl which is substituted or unsubstituted, or together with any of L², R^{a}, R^{b}, and R^{d} forms a ring that is substituted or unsubstituted; R^{d} is H or alkyl which is substituted or unsubstituted, or together with any of L², R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted.

In some embodiments, the Tie-2 modulator forms at least about 99.0% (w/w) of the mixture, and wherein the second compound forms from about 0.001% to about 0.5%, from about 0.001% to about 0.1% (w/w), from about 0.01% to about 0.1% (w/w), or from about 0.01% to about 0.03% (w/w) of the mixture.

In some embodiments, Aryl² is substituted or unsubstituted heteroaryl; and X is alkylene.

In some embodiments, Aryl² is substituted heteroaryl; and X is methylene.

In some embodiments, each Z is:

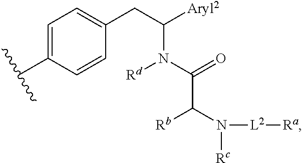

wherein:
Aryl² is substituted heteroaryl; L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, Aryl² is a substituted thiazole moiety; L² together with the nitrogen atom to which L² is bound forms a carbamate linkage; R^{a} is alkyl, which is substituted or unsubstituted; R^{b} is arylalkyl, which is substituted or unsubstituted; R^{c} is H; and R^{d} is H.

In some embodiments, Aryl² is:

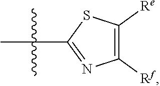

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, R^{a} is alkyl, which is substituted or unsubstituted; R^{b} is arylalkyl, which is substituted or unsubstituted; R^{e} is H; and R^{f} is heteroaryl.

In some embodiments, each Z is:

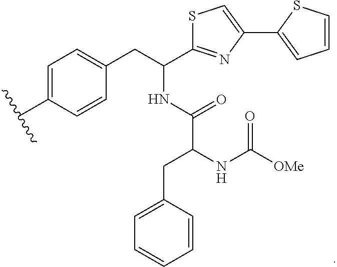 [SMILES: *c1ccc(CC(NC(=O)C(Cc2ccccc2)NC(=O)OC)c2nc(-c3cccs3)cs2)cc1]

In some embodiments, each Z is:

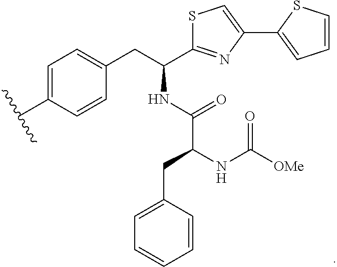 [SMILES: *c1ccc(C[C@H](NC(=O)[C@H](Cc2ccccc2)NC(=O)OC)c2nc(-c3cccs3)cs2)cc1]

In some embodiments, Aryl² is:

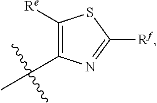

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, R^{a} is alkyl, which is substituted or unsubstituted; R^{b} is arylalkyl, which is substituted or unsubstituted; R^{e} is H; and R^{f} is alkyl.

In some embodiments, each Z is:

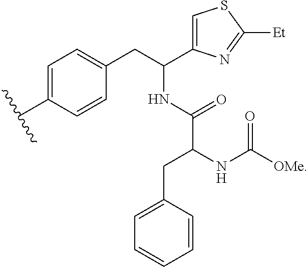 [SMILES: *c1ccc(CC(NC(=O)C(Cc2ccccc2)NC(=O)OC)c2csc(CC)n2)cc1]

In some embodiments, each Z is:

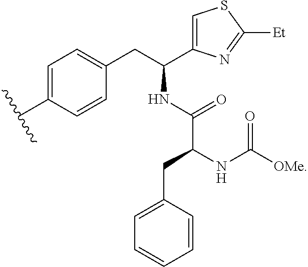 [SMILES: *c1ccc(C[C@H](NC(=O)[C@H](Cc2ccccc2)NC(=O)OC)c2csc(CC)n2)cc1]

In some embodiments, R^{a} is alkyl, which is substituted or unsubstituted; R^{b} is arylalkyl, which is substituted or unsubstituted; R^{e} is H; and R^{f} is heteroaryl.

In some embodiments, each Z is:

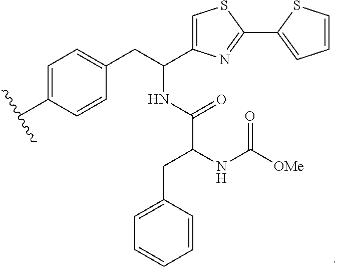 [SMILES: *c1ccc(CC(NC(=O)C(Cc2ccccc2)NC(=O)OC)c2csc(-c3cccs3)n2)cc1]

In some embodiments, each Z is:

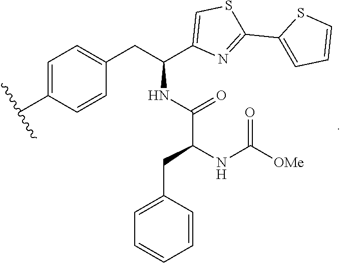 [SMILES: *c1ccc(C[C@H](NC(=O)[C@H](Cc2ccccc2)NC(=O)OC)c2csc(-c3cccs3)n2)cc1]

In some embodiments, the Tie-2 modulator forms at least about 99.2%, at least about 99.3%, at least about 99.4%, at least about 99.5%, at least about 99.6%, at least about 99.7%, or at least about 99.8% (a/a) of the mixture as determined by a liquid chromatography assay.

In some embodiments, the Tie-2 modulator forms from about 99.3% to about 99.5%, about 99.3% to about 99.6%, or about 99.3% to about 99.7% (a/a) of the mixture as determined by a liquid chromatography assay.

In some embodiments, the second compound forms from about 0.001% to about 0.10%, about 0.01% to about 0.10%, or about 0.01% to about 0.03% (a/a) of the mixture as determined by a liquid chromatography assay.

In some embodiments, the Tie-2 modulator is a Tie-2 activator. In some embodiments, the Tie-2 modulator is a HPTPβ inhibitor.

### Azoxy Compound.

In some embodiments, the disclosure provides an azoxy compound that has a structure of U-J-U, wherein J is

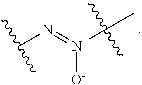 [SMILES: */N=[N+](/*)[O-]]

In some embodiments, each U is:

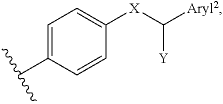

wherein
Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; and Y is H, aryl, heteroaryl, NH(aryl), NH(heteroaryl), or NHCOR^{g}, any of which is substituted or unsubstituted, or

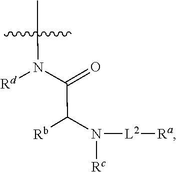

wherein:
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted; R^{c} is H or alkyl which is substituted or unsubstituted, or together with any of L², R^{a}, R^{b}, and R^{d} forms a ring that is substituted or unsubstituted; R^{d} is H or alkyl which is substituted or unsubstituted, or together with any of L², R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted.

In some embodiments, Aryl² is substituted or unsubstituted heteroaryl; and X is alkylene.

In some embodiments, Aryl² is substituted heteroaryl; and X is methylene.

In some embodiments, each U is:

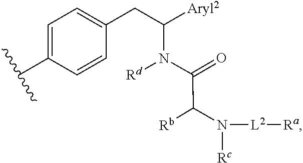

wherein:
Aryl² is substituted heteroaryl; L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, Aryl² is a substituted thiazole moiety; L² together with the nitrogen atom to which L² is bound forms a carbamate linkage; R^{a} is alkyl, which is substituted or unsubstituted; R^{b} is arylalkyl, which is substituted or unsubstituted; R^{c} is H; and R^{d} is H.

In some embodiments, Aryl² is:

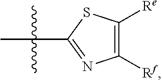

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, R^{a} is alkyl, which is substituted or unsubstituted; R^{b} is arylalkyl, which is substituted or unsubstituted; R^{e} is H; and R^{f} is heteroaryl.

In some embodiments, each U is:

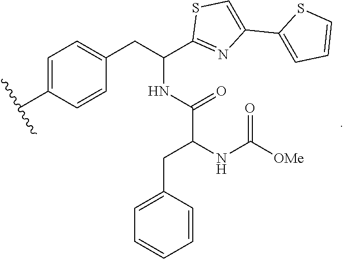 [SMILES: *c1ccc(CC(NC(=O)C(Cc2ccccc2)NC(=O)OC)c2nc(-c3cccs3)cs2)cc1]

In some embodiments, each U is:

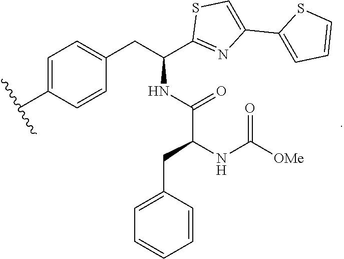 [SMILES: *c1ccc(C[C@H](NC(=O)[C@H](Cc2ccccc2)NC(=O)OC)c2nc(-c3cccs3)cs2)cc1]

In some embodiments, Aryl² is:

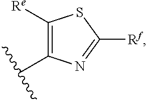

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted.

In some embodiments, R^{a} is alkyl, which is substituted or unsubstituted; R^{b} is arylalkyl, which is substituted or unsubstituted; R^{e} is H; and R^{f} is alkyl.

In some embodiments, each U is:

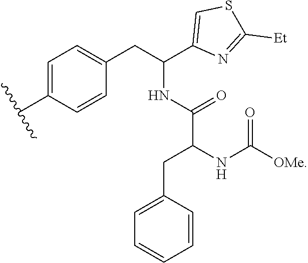 [SMILES: *c1ccc(CC(NC(=O)C(Cc2ccccc2)NC(=O)OC)c2csc(CC)n2)cc1]

In some embodiments, each U is:

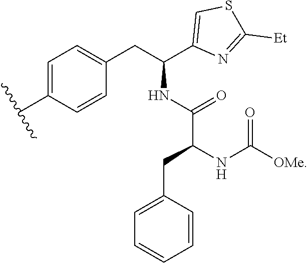 [SMILES: *c1ccc(C[C@H](NC(=O)[C@H](Cc2ccccc2)NC(=O)OC)c2csc(CC)n2)cc1]

In some embodiments, R^{a} is alkyl, which is substituted or unsubstituted; R^{b} is arylalkyl, which is substituted or unsubstituted; R^{e} is H; and R^{f} is heteroaryl.

In some embodiments, each U is:

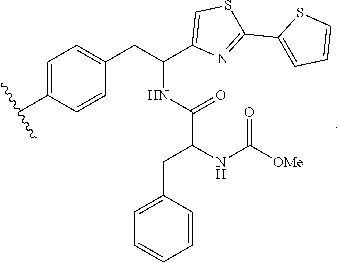 [SMILES: *c1ccc(CC(NC(=O)C(Cc2ccccc2)NC(=O)OC)c2csc(-c3cccs3)n2)cc1]

In some embodiments, each U is:

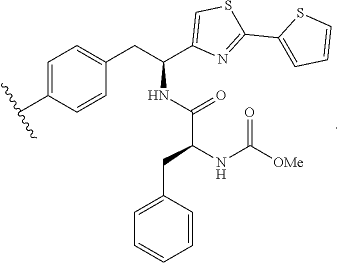 [SMILES: *c1ccc(C[C@H](NC(=O)[C@H](Cc2ccccc2)NC(=O)OC)c2csc(-c3cccs3)n2)cc1]

In some embodiments, the azoxy compound is

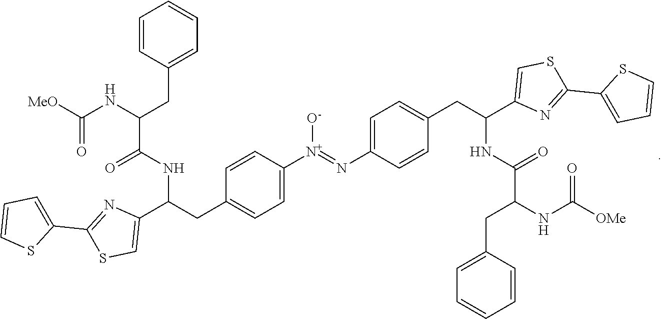 [SMILES: COC(=O)NC(Cc1ccccc1)C(=O)NC(Cc1ccc(/N=[N+](\[O-])c2ccc(CC(NC(=O)C(Cc3ccccc3)NC(=O)OC)c3csc(-c4cccs4)n3)cc2)cc1)c1csc(-c2cccs2)n1]

In some embodiments, the azoxy compound is

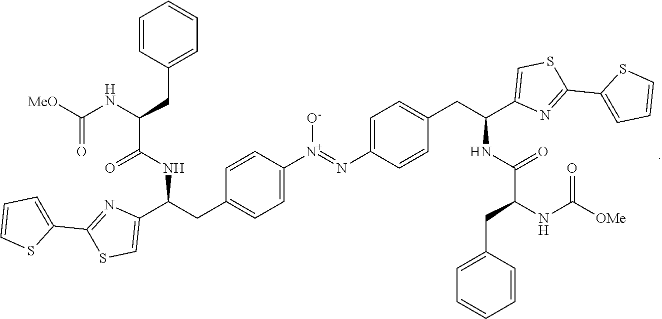 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

### Formulations.

A pharmaceutical composition of the disclosure can provide a therapeutically-effective amount of an inhibitor of HPTPβ. A pharmaceutical composition of the disclosure can provide a therapeutically-effective amount of an activator of Tie-2.

The disclosed formulations can comprise one or more pharmaceutically-acceptable agents, which alone or in combination can solubilize a compound herein or a pharmaceutically-acceptable salt thereof.

In some embodiments, a compound or pharmaceutically-acceptable salt thereof is present in a formulation in an amount of from about 0.1 mg/mL to about 100 mg/mL, from about 0.1 mg/mL to about 1 mg/mL, from about 0.1 mg/mL to about 5 mg/mL, from about 5 mg/mL to about 10 mg/mL, from about 10 mg/mL to about 15 mg/mL, from about 15 mg/mL to about 20 mg/mL, from about 20 mg/mL to about 25 mg/mL, from about 25 mg/mL to about 30 mg/mL, from about 30 mg/mL to about 35 mg/mL, from about 35 mg/mL to about 40 mg/mL, from about 40 mg/mL to about 45 mg/mL, about 45 mg/mL to about 50 mg/mL, from about 50 mg/mL to about 55 mg/mL, from about 55 mg/mL to about 60 mg/mL, from about 60 mg/mL to about 65 mg/mL, from about 65 mg/mL to about 70 mg/mL, from about 70 mg/mL to about 75 mg/mL, about 75 mg/mL to about 80 mg/mL, from about 80 mg/mL to about 85 mg/mL, from about 85 mg/mL to about 90 mg/mL, from about 90 mg/mL to about 95 mg/mL, or from about 95 mg/mL to about 100 mg/mL.

In some embodiments, a compound or pharmaceutically-acceptable salt thereof is present in a formulation in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL about 72 mg/mL, about 73 mg/mL, about 74 mg/mL, about 75 mg/mL, about 76 mg/mL, about 77 mg/mL, about 78 mg/mL, about 79 mg/mL, about 80 mg/mL, about 81 mg/mL about 82 mg/mL, about 83 mg/mL, about 84 mg/mL, about 85 mg/mL, about 86 mg/mL, about 87 mg/mL, about 88 mg/mL, about 89 mg/mL, about 90 mg/mL, about 91 mg/mL, about 92 mg/mL, about 93 mg/mL, about 94 mg/mL, about 95 mg/mL, about 96 mg/mL, about 97 mg/mL, about 98 mg/mL, about 99 mg/mL, or about 100 mg/mL.

A formulation that is disclosed herein can be made more soluble by the addition of an additive or agent, for example, a cyclodextrin moiety. The improvement of solubility of the formulation can increase by about 5%, about 10%, about 15%, about 20%, about 25%, about 30%, about 35%, about 40%, about 45%, about 50%, about 55%, about 60%, about 65%, about 70%, about 75% about 80%, about 85%, about 90%, about 95%, about 100%, about 110%, about 120%, about 130%, about 140%, about 150%, about 160%, about 170%, about 180%, about 190%, about 200%, about 225%, about 250%, about 275%, about 300%, about 325%, about 350%, about 375%, about 400%, about 450%, or about 500%.

A formulation disclosed herein can be stable for about 1 day, about 2 days, about 3 days, about 4 days, about 5 days, about 6 days, about 7 days, about 8 days, about 9 days, about 10 days, about 2 weeks, about 4 weeks, about 6 weeks, about 8 weeks, about 10 weeks, about 12 weeks, about 3 months, about 4 months, about 5 months, about 6 months, about 7 months, about 8 months, about 9 months, about 10 months, about 11 months, or about one year. A formulation disclosed herein can be stable, for example, at about 0° C., about 5° C., about 10° C., about 15° C., about 20° C., about 25° C., about 30° C., about 35° C., about 40° C., about 45° C., about 50° C., about 60° C., about 70° C., or about 80° C.

### Alcohols.

A non-limiting example of a solubilizing agent includes an organic solvent. Non-limiting examples of organic solvents include alcohols, for example, C₁-C₄ linear alkyl, C₃-C₄ branched alkyl, ethanol, glycerin, 2-hydroxypropanol, propylene glycol, maltitol, sorbitol, xylitol; substituted or unsubstituted aryl, and benzyl alcohol.

### Cyclodextrins.

Non-limiting examples of cyclodextrins include β-cyclodextrin, methyl β-cyclodextrin, 2-hydroxypropyl-β-cyclodextrin (HPβCD), hydroxyethyl-β-cyclodextrin (HE-β-CD), heptakis (2,6-di-O-methyl)-β-cyclodextrin (DMPCD), α-cyclodextrin, γ-cyclodextrin, 2-hydroxypropyl-γ-cyclodextrin (HPγCD), and sulfobutylether-β-cyclodextrin (SBECD) sodium salt. A cyclodextrin can possess a large cyclic structure with a channel passing through the center of the structure. The interior of the cyclodextrin can be hydrophobic, and interact favorably with hydrophobic molecules. The exterior of the cyclodextrin can be highly hydrophilic owing to the several hydroxyl groups exposed to bulk solvent. Capture of a hydrophobic molecule, such as a compound disclosed herein, in the channel of the cyclodextrin can result in the formation of a complex stabilized by non-covalent hydrophobic interactions. The complex can be soluble in water, and carry the captured hydrophobic molecule into the bulk solvent.

Formulations of the disclosure can comprise randomly methylated β-cyclodextrins (RAMEB or RMCD). The formulations of the disclosure can comprise RAMEB comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21 methyl groups.

The disclosed solubilizing systems comprise 2-hydroxypropyl-beta-cyclodextrin (HPβCD). 2-Hydroxypropyl-β-cyclodextrin [CAS No. 128446-35-5] is commercially available as Cavitron™. 2-Hydroxypropyl-β-cyclodextrin, also described known as hydroxypropyl-β-cyclodextrin, 2-hydroxypropyl-beta-cyclodextrin, hydroxypropyl-beta-cyclodextrin or HPβCD, can be represented by either of the following formulae:

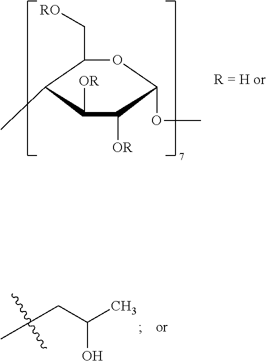

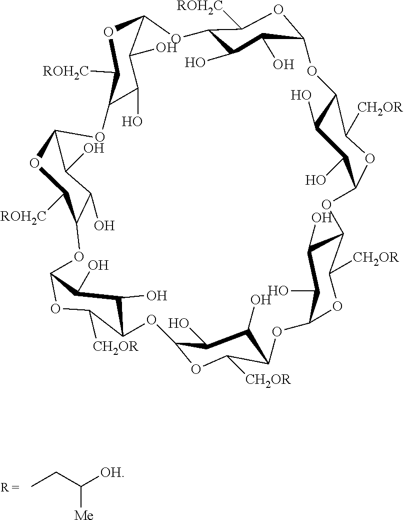

The average molecular weight of Cavitron™, is approximately 1396 Da, wherein the average degree of substitution is from about 0.5 to about 1.3 units of 2-hydroxypropyl per ring glucose unit.

In one embodiment, a formulation disclosed herein can comprise a ratio of about 20 parts of a compound herein or a pharmaceutically-acceptable salt thereof to about 1 part solubilizing system (about 20:about 1), to about 1 part of the compound herein or a pharmaceutically-acceptable salt thereof to about 20 parts solubilizing system (about 1:about 20). For example, a formulation containing about 100 mg of a compound herein or a pharmaceutically-acceptable salt thereof can contain from about 5 mg to about 2000 mg of a solubilizing agent, such as a cyclodextrin. In another embodiment, the ratio can be based on number, or moles, or compound compared to number, or moles, of the solubilizing system.

The following are non-limiting examples of ratios of a compound herein and a solubilizing agent, such as a cyclodextrin. The following examples alternatively describe the ratio of a solubilizing agent, such as a cyclodextrin, and a compound herein. The ratio can be: about 20:about 1; about 19.9:about 1; about 19.8:about 1; about 19.7:about 1; about 19.6:about 1; about 19.5:about 1; about 19.4:about 1; about 19.3:about 1; about 19.2:about 1; about 19.1:about 1; about 19:about 1; about 18.9:about 1; about 18.8:about 1; about 18.7:about 1; about 18.6:about 1; about 18.5:about 1; about 18.4:about 1; about 18.3:about 1; about 18.2:about 1; about 18.1:about 1; about 18:about 1; about 17.9:about 1; about 17.8:about 1; about 17.7:about 1; about 17.6:about 1; about 17.5:about 1; about 17.4:about 1; about 17.3:about 1; about 17.2:about 1; about 17.1:about 1; about 17:about 1; about 16.9:about 1; about 16.8:about 1; about 16.7:about 1; about 16.6:about 1; about 16.5:about 1; about 16.4:about 1; about 16.3:about 1; about 16.2:about 1; about 16.1:about 1; about 16:about 1; about 15.9:about 1; about 15.8:about 1; about 15.7:about 1; about 15.6:about 1; about 15.5:about 1; about 15.4:about 1; about 15.3:about 1; about 15.2:about 1; about 15.1:about 1; about 15:about 1; about 14.9:about 1; about 14.8:about 1; about 14.7:about 1; about 14.6:about 1; about 14.5:about 1; about 14.4:about 1; about 14.3:about 1; about 14.2:about 1; about 14.1:about 1; about 14:about 1; about 13.9:about 1; about 13.8:about 1; about 13.7:about 1; about 13.6:about 1; about 13.5:about 1; about 13.4:about 1; about 13.3:about 1; about 13.2:about 1; about 13.1:about 1; about 13:about 1; about 12.9:about 1; about 12.8:about 1; about 12.7:about 1; about 12.6:about 1; about 12.5:about 1; about 12.4:about 1; about 12.3:about 1; about 12.2:about 1; about 12.1:about 1; about 12:about 1; about 11.9:about 1; about 11.8:about 1; about 11.7:about 1; about 11.6:about 1; about 11.5:about 1; about 11.4:about 1; about 11.3:about 1; about 11.2:about 1; about 11.1:about 1; about 11:about 1; about 10.9:about 1; about 10.8:about 1; about 10.7:about 1; about 10.6:about 1; about 10.5:about 1; about 10.4:about 1; about 10.3:about 1; about 10.2:about 1; about 10.1:about 1; about 10:about 1; about 9.9:about 1; about 9.8:about 1; about 9.7:about 1; about 9.6:about 1; about 9.5:about 1; about 9.4:about 1; about 9.3:about 1; about 9.2:about 1; about 9.1 about 1; about 9:about 1; about 8.9:about 1; about 8.8:about 1; about 8.7:about 1; about 8.6:about 1; about 8.5:about 1; about 8.4:about 1; about 8.3:about 1; about 8.2:about 1; about 8.1:about 1; about 8:about 1; about 7.9:about 1; about 7.8:about 1; about 7.7 about 1; about 7.6:about 1; about 7.5:about 1; about 7.4:about 1; about 7.3:about 1; about 7.2:about 1; about 7.1:about 1; about 7:about 1; about 6.9:about 1; about 6.8 about 1; about 6.7:about 1; about 6.6:about 1; about 6.5:about 1; about 6.4:about 1; about 6.3:about 1; about 6.2:about 1; about 6.1:about 1; about 6:about 1; about 5.9 about 1; about 5.8:about 1; about 5.7:about 1; about 5.6:about 1; about 5.5:about 1; about 5.4:about 1; about 5.3:about 1; about 5.2:about 1; about 5.1:about 1; about 5 about 1; about 4.9:about 1; about 4.8:about 1; about 4.7:about 1; about 4.6:about 1; about 4.5:about 1; about 4.4:about 1; about 4.3:about 1; about 4.2:about 1; about 4.1 about 1; about 4:about 1; about 3.9:about 1; about 3.8:about 1; about 3.7:about 1; about 3.6:about 1; about 3.5:about 1; about 3.4:about 1; about 3.3:about 1; about 3.2:about 1; about 3.1:about 1; about 3:about 1; about 2.9:about 1; about 2.8:about 1; about 2.7:about 1; about 2.6:about 1; about 2.5:about 1; about 2.4:about 1; about 2.3:about 1; about 2.2:about 1; about 2.1:about 1; about 2:about 1; about 1.9:about 1; about 1.8:about 1; about 1.7:about 1; about 1.6:about 1; about 1.5:about 1; about 1.4:about 1; about 1.3:about 1; about 1.2:about 1; about 1.1:about 1; or about 1:about 1.

### Polyvinylpyrrolidone.

Another non-limiting example of a solubilizing agent is polyvinylpyrrolidone (PVP), having the formula:

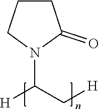

wherein the index n is from about 40 to about 200. PVP's can have an average molecular weight from about 5500 to about 28,000 g/mol. One non-limiting example is PVP-10, having an average molecular weight of approximately 10,000 g/mol.

### Polyalkyleneoxides and Ethers Thereof.

Another non-limiting example of solubilizing agents includes polyalkyleneoxides, and polymers of alcohols or polyols. Polymers can be mixed, or contain a single monomeric repeat subunit. For example, polyethylene glycols having an average molecular weight of from about 200 to about 20,000, for example, PEG 200, PEG 400, PEG 600, PEG 1000, PEG 1450, PEG 1500, PEG 4000, PEG 4600, and PEG 8000. In a same embodiment, a composition comprises one or more polyethylene glycols chosen from PEG 400, PEG 1000, PEG 1450, PEG 4600 and PEG 8000.

Other polyalkyleneoxides are polypropylene glycols having the formula:

HO[CH(CH₃)CH₂O]ₓH
wherein the index x represents the average number of propyleneoxy units in the polymer. The index x can be represented by a whole number or a fraction. For example, a polypropylene glycol having an average molecular weight of 8,000 g/mole (PEG 8000) can be represented by the formulae:

HO[CH(CH₃)CH₂O]₁₃₈H or HO[CH(CH₃)CH₂O]_{137.6}H
or the polypropylene glycol can be represented by the common, short hand notation: PPG 8000.

Another example of polypropylene glycols can have an average molecular weight from about 1200 g/mol to about 20,000 g/mol, i.e., a polypropylene glycol having an average molecular weight of about 8,000 g/mol, for example, PPG 8000.

Another solubilizing agent is Polysorbate 80 (Tween™ 80), which is an oleate ester of sorbitol and its anhydrides copolymerized with approximately 20 moles of ethylene oxide for each mole of sorbitol and sorbitol anhydrides. Polysorbate 80 is made up of sorbitan mono-9-octadecanoate poly(oxy-1,2-ethandiyl) derivatives.

Solubilizing agents also include poloxamers having the formula:

HO(CH₂CH₂)_{y1}(CH₂CH₂CH₂O)_{y2}(CH₂CH₂O)_{y3}OH

which are nonionic block copolymers composed of a polypropyleneoxy unit flanked by two polyethyleneoxy units. The indices y¹, y², and y³ have values such that the poloxamer has an average molecular weight of from about 1000 g/mol to about 20,000 g/mol.

### Excipients.

A pharmaceutical composition of the disclosure can be a combination of any pharmaceutical compounds described herein with other chemical components, such as carriers, stabilizers, diluents, dispersing agents, suspending agents, thickening agents, or excipients. The pharmaceutical composition facilitates administration of the compound to an organism. Pharmaceutical compositions can be administered in therapeutically-effective amounts as pharmaceutical compositions by various forms and routes including, for example, intravenous, intravitreal, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, suspensions, and the like, for oral ingestion by a subject.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient such as gum arabic, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, or lubricants such as talc or magnesium stearate and, stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parenteral injections can be formulated for bolus injection or continuous infusion. The pharmaceutical compositions can be in a form suitable for parenteral injection as a sterile suspension, solution, or emulsion in oily or aqueous vehicles, and can contain formulatory agents such as suspending, stabilizing, or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on demand delivery of pharmaceutical compounds. Transdermal delivery can be accomplished by means of iontophoretic patches. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a mist, or a powder. Pharmaceutical compositions are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compounds and a suitable powder base such as lactose or starch.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone and PEG. In suppository forms of the compositions, a low-melting wax such as a mixture of fatty acid glycerides or cocoa butter can be used.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, granulating, dragee-making, levigating, emulsifying, encapsulating, entrapping, or compression processes.

The pharmaceutical compositions can include at least one pharmaceutically-acceptable carrier, diluent, or excipient and compounds described herein as free-acids or pharmaceutically-acceptable salt forms. The methods and pharmaceutical compositions described herein include the use of crystalline forms (also known as polymorphs), and active metabolites of these compounds having the same type of activity.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the present disclosure include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

The disclosure can be administered as an eye drop. The average volume of each drop administered to a subject can be about 5 μl, about 10 μl, about 15 μl, about 20 μl, about 30 μl, about 40 μl, about 50 μl, about 60 μl, about 70 μl, about 80 μl, about 90 μl, or about 100 μl. The eye drops can contain about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 10.5%, about 11%, about 11.5%, about 12%, about 120.5%, about 13%, about 130.5%, about 14%, about 140.5%, about 15%, about 15.5%, about 16%, about 160.5%, about 17%, about 170.5%, about 18%, about 180.5%, about 19%, about 190.5%, or about 20% of a compound of the disclosure. The drops can contain about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 60 mg/ml, about 70 mg/ml, about 80 mg/ml, about 90 mg/ml, about 100 mg/ml, about 120 mg/ml, about 140 mg/ml, about 160 mg/ml, about 180 mg/ml, or about 200 mg/ml of a compound of the disclosure. The individual dose administered to a subject can be about 0.5 μg, about 1 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1 mg, about 1.1 mg, about 1.2 mg, 1.3 mg, about 1.4 mg, about 1.5 mg, about 1.6 mg, about 1.7 mg, about 1.8 mg, about 1.9 mg, or about 2 mg of a compound of the disclosure. In some embodiments, more than one drop can be administered to an eye either at one time or at multiple times throughout the day.

Non-limiting examples of excipients suitable for use in eye drops in the present disclosure include cyclodextrin, α-cyclodextrin, β-cyclodextrin, 2-hydroxypropyl-β-cyclodextrin (HP-β-CD), random methyl-β-cyclodextrin (RM-β-CD), sulfobutyl ether β-cyclodextrin (SBE-β-CD), γ-cyclodextrin, hydroxypropyl-γ-cyclodextrin (HP-γ-CD), hydroxyethyl-β-cyclodextrin (HE-β-CD), heptakis (2,6-di-O-methyl)-β-cyclodextrin (DMPCD), saline, sodium bisulfate, metabisulfate, ascorbic acid, acetylcysteine, benzalkonium chloride, boric acid, hyaluronic acid, hypromellose, propylene glycol, potassium sorbate, sodium chloride, sodium acetate, disodium edetate, sodium dihydrogen phosphate monohydrate, disodium phosphate, sodium hydroxide, hydrochloric acid, glycerol, mannitol, trometamol, tyloxapol, and any combination thereof.

The individual dose administered to a subject can be about 0.5 mg, about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg of a compound of the present disclosure. The individual dose administered to a subject can be from about 0.1 mg to about 25 mg, about 0.1 mg to about 50 mg, about 0.1 mg to about 75 mg, or about 0.1 mg to about 100 mg. The individual dose administered to a subject can be from about 0.5 mg to about 10 mg, about 0.5 mg to about 20 mg, or about 0.5 mg to about 30 mg. In some embodiments, the individual dose administered to a subject can be about 10 mg of a compound of the present disclosure. In some embodiments, the individual dose administered to a subject can be about 15 mg of a compound of the present disclosure. In some embodiments, the individual dose administered to a subject can be about 20 mg of a compound of the present disclosure. In some embodiments, the individual dose administered to a subject can be about 30 mg of a compound of the present disclosure. In some embodiments, the individual dose of a compound of the present disclosure administered to a subject can be about 15 mg twice per day or about 30 mg per day.

Non-limiting examples of pharmaceutically-acceptable excipients suitable for use in the present disclosure include granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, anti-microbial agents, plant cellulosic material and spheronization agents, and any combination thereof.

A composition of the present disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

The disclosed compositions can optionally comprise from about 0.001% to about 0.005% weight by volume pharmaceutically-acceptable preservatives. One non-limiting example of a suitable preservative is benzyl alcohol.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

The disclosed methods include administration of an HPTPβ inhibitor, or a pharmaceutically-acceptable salt thereof, in combination with a pharmaceutically-acceptable carrier. The carrier can be selected to minimize any degradation of the active ingredient and to minimize any adverse side effects in the subject.

The disclosed methods include administration of a Tie-2 activator, or a pharmaceutically-acceptable salt thereof, in combination with a pharmaceutically-acceptable carrier. The carrier can be selected to minimize any degradation of the active ingredient and to minimize any adverse side effects in the subject.

The Tie-2 activator or a pharmaceutically-acceptable salt thereof herein can be conveniently formulated into pharmaceutical compositions composed of one or more pharmaceutically-acceptable carriers. See e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the compound described herein and which is incorporated by reference herein. Such pharmaceuticals can be standard carriers for administration of compositions to humans and non-humans, including solutions such as sterile water, saline, and buffered solutions at physiological pH. Other compositions can be administered according to standard procedures. For example, pharmaceutical compositions can also include one or more additional active ingredients such as antimicrobial agents, anti-inflammatory agents, and anesthetics.

Non-limiting examples of pharmaceutically-acceptable carriers include saline solution, Ringer's solution, and dextrose solution. The pH of the solution can be from about 5 to about 8, and can be from about 7 to about 7.5. Further carriers include sustained release preparations such as semipermeable matrices of solid hydrophobic polymers containing the Tie-2 activator or a pharmaceutically-acceptable salt thereof, where the matrices are in the form of shaped articles, such as films, liposomes, microparticles, and microcapsules.

The disclosed methods relate to administering the Tie-2 activator or a pharmaceutically-acceptable salt thereof as part of a pharmaceutical composition. The disclosed methods relate to administering the HPTPβ inhibitor or a pharmaceutically-acceptable salt thereof as part of a pharmaceutical composition. In various embodiments, compositions of the present disclosure can comprise a liquid comprising an active agent in solution, in suspension, or both. Liquid compositions can include gels. In one embodiment, the liquid composition is aqueous. Alternatively, the composition can take form of an ointment. In another embodiment, the composition is an in situ gellable aqueous composition. In some embodiments, the composition is an in situ gellable aqueous solution.

Pharmaceutical formulations can include additional carriers, as well as thickeners, diluents, buffers, preservatives, and surface active agents in addition to the compounds disclosed herein. Pharmaceutical formulations can also include one or more additional active ingredients such as antimicrobial agents, anti-inflammatory agents, or anesthetics.

An excipient can fill a role as simple and direct as being an inert filler, or an excipient as used herein can be part of a pH stabilizing system or coating to ensure delivery of the ingredients safely to the stomach.

The Tie-2 activator or a pharmaceutically-acceptable salt thereof can also be present in liquids, emulsions, or suspensions for delivery of active therapeutic agents in aerosol form to cavities of the body such as the nose, throat, or bronchial passages. The ratio of Tie-2 activator or a pharmaceutically-acceptable salt thereof to the other compounding agents in these preparations can vary as the dosage form requires.

Depending on the intended mode of administration, the pharmaceutical compositions administered as part of the disclosed methods can be in the form of solid, semi-solid or liquid dosage forms, such as, for example, tablets, suppositories, pills, capsules, powders, liquids, suspensions, lotions, creams, gels, for example, in unit dosage form suitable for single administration of a precise dosage. The compositions can contain, as noted above, an effective amount of the Tie-2 activator or a pharmaceutically-acceptable salt thereof in combination with a pharmaceutically-acceptable carrier and, in addition, can include other medicinal agents, pharmaceutical agents, carriers, adjuvants, diluents, etc.

For solid compositions, nontoxic solid carriers include, for example, pharmaceutical grades of mannitol, lactose, starch, magnesium stearate, sodium saccharin, talc, cellulose, glucose, sucrose, and magnesium carbonate. In one embodiment, a composition comprising the Tie-2 activator or a pharmaceutically-acceptable salt thereof in an amount of approximately 4 mg per 0.1 mL liquid is prepared. The liquid phase comprises sterile water and an appropriate amount of a saccharide or polysaccharide.

### Pharmaceutical Compositions.

Pharmaceutical compositions containing the compounds described herein can be administered for prophylactic or therapeutic treatments. Compositions can contain any number of active agents. In therapeutic applications, the compositions can be administered to a subject already suffering from a disease or condition, in an amount sufficient to cure or at least partially arrest the symptoms of the disease or condition, or to cure, heal, improve, reduce, lessen, ameliorate, or reduce a likelihood of the disease or condition. Compounds can also be administered to lessen or reduce a likelihood of developing, contracting, or worsening a condition. Amounts effective for this use can vary based on the severity and course of the disease or condition, previous therapy, the subject's health status, weight, response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills or injections. The compounds can be packed together or separately, in a single package or in a plurality of packages. One or all of the therapeutic agents can be given in multiple doses. If not simultaneous, then the timing between the multiple doses can vary.

Compounds and compositions described herein can be packaged as a kit. In some embodiments, the present disclosure provides a kit comprising a compound disclosed herein, or a pharmaceutically-acceptable salt thereof, and written instructions on use of the kit in the treatment of a condition described herein. In some embodiments, the present disclosure provides a kit comprising a compound disclosed herein, or a pharmaceutically-acceptable salt thereof, an antibody, and written instructions on use of the kit in the treatment of a condition described herein.

The compounds described herein can be administered before, during, or after the occurrence of a disease or condition, and the timing of administering the composition containing a compound can vary. For example, the compounds can be used as a prophylactic and can be administered continuously to subjects with a propensity to conditions or diseases to lessen or reduce a likelihood of the occurrence of the disease or condition. The compounds and compositions can be administered to a subject during or as soon as possible after the onset of the symptoms. The administration of the compounds can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, a compound disclosed herein is administered for the lifetime of a subject. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

A Tie-2 activator described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, or from about 250 mg to about 300 mg.

A Tie-2 activator described herein can be present in a composition in an amount of about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, or about 300 mg.

Treatment of Subjects with a Tie-2 Activator.

The present disclosure provides methods for treating a subject afflicted with vascular disorders with an activator of Tie-2 or an inhibitor of HPTPβ. The subject can be a human. Treatment can include treating a human in a clinical trial. A treatment can comprise administering to a subject a pharmaceutical composition comprising one or more of the activators of Tie-2 described throughout the disclosure. A treatment can comprise administrating to a subject a therapy that promotes the phosphorylation of a Tie-2 molecule.

The present disclosure provides methods for treating a subject afflicted with vascular disorders with a therapeutically-effective amount of an activator of Tie-2 or an inhibitor of HPTPβ. The subject can be a human. Treatment can include treating a human in a clinical trial. A treatment can comprise administering to a subject a pharmaceutical composition comprising one or more of the activators of Tie-2 described throughout the disclosure. A treatment can comprise administering to a subject a therapy that promotes the phosphorylation of a Tie-2 molecule. A therapeutically-effective amount can be from about 0.1 mg to about 100 mg or from about 0.5 mg to about 30 mg.

Non-limiting examples of possible subjects for administration include the following. Subjects can be humans, non-human primates such as chimpanzees, and other apes and monkey species; farm animals such as cattle, horses, sheep, goats, and swine; domestic animals such as rabbits, dogs, and cats; and laboratory animals including rats, mice, and guinea pigs. A subject can be of any age. Subjects can be, for example, elderly adults, adults, adolescents, pre-adolescents, children, toddlers, and infants.

A subject described herein can express Ang-1. A subject described herein can express Ang-2. A subject described herein can express both Ang-1 and Ang-2.

### EXAMPLES

### Example 1: Preparation of Intermediate F-2

### Preparation of (methoxycarbonyl)-L-phenylalanine (F-2)

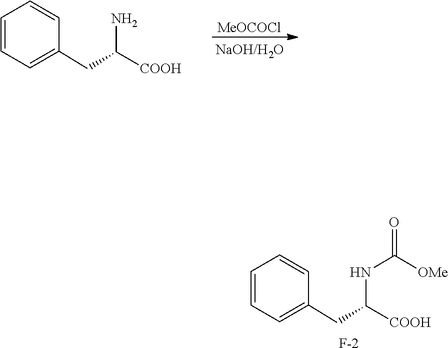

L-phenylalanine (13.6 kg) was suspended in water (39.2 kg), and the suspension was cooled to 3° C. under a nitrogen atmosphere. 30% (w/w) aqueous sodium hydroxide (33.3 kg) was then added via an addition line, and the temperature was maintained under 15° C. during the addition. Water (3.4 kg) was used to rinse any remaining sodium hydroxide solution from the addition line into the reaction vessel. The reaction mixture was cooled to −12° C., and methyl chloroformate (10.6 kg) was then slowly added to the reaction mixture over 2 hours. The temperature of the reaction mixture was maintained below 0° C. throughout the addition. After completion of the addition, MTBE (4.8 kg) was used to rinse any remaining methyl chloroformate from the addition line into the reaction vessel. After 20 minutes of stirring at 0° C., a sample was withdrawn and submitted for UPLC analysis (Method 1, EXAMPLE 6). Side-product G-1 was not detected, and 8% of the L-phenylalanine remained in the reaction mixture.

After stirring for a further 8.5 hours at 0° C., MTBE (37.4 kg) was added, and the reaction was allowed to warm to 18° C. over 1 hour. 15.5 kg of 37% (w/w) hydrochloric acid was added over 1.5 hours, and the temperature was maintained below 25° C. throughout the addition. After completion of the addition, nitrogen gas and water (10 kg) were used to rinse any remaining hydrochloric acid solution from the addition line into the reaction vessel, and the mixture was stirred for 10 minutes. The pH of the reaction mixture was determined to be 0 via colorimetric strips. The reaction was allowed to warm to 22° C. over 30 minutes and stirred for 1 hour, after which time stirring was stopped and the contents were allowed to settle for 40 minutes. The aqueous phase was drained from the biphasic mixture, and 25% aqueous sodium chloride (32.1 kg) was then added to the remaining organic phase. The mixture was stirred for 40 minutes, after which time stirring was stopped and the contents were allowed to settle for 30 minutes. The aqueous phase was then drained from the mixture. MTBE (81.6 kg) was then added to the remaining organic phase, and the resulting mixture was concentrated to 55 liters via distillation at 60° C. at ambient pressure. The water content of the concentrate was 0.9% (w/w) as determined by Karl Fischer analysis. The concentrate was filtered, and MTBE (23.7 kg) was used to rinse any remaining concentrate from the reaction vessel and filter into the filtrate receptacle to provide 70 kg of a 24.1% w/w solution of F-2 in MTBE, which was directly used in the next synthetic step.

### Dimer G-1 Formation Study.

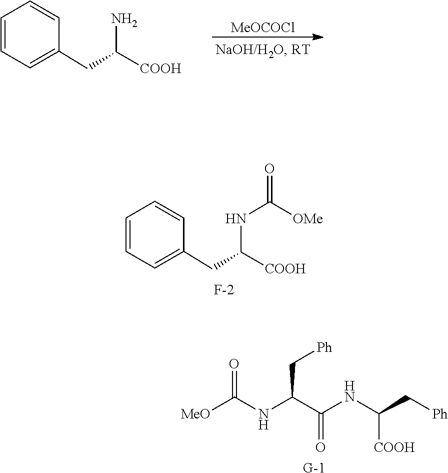

Addition of an excess of methyl chloroformate at temperatures above 0° C. can lead to formation of side-product G-1. Two portions of 1.33 eq. of methyl chloroformate were added to a solution of L-phenylalanine in aqueous NaOH (30% w/w) at room temperature. The reaction temperature rose from 20° C. to 39° C. during the addition. Analysis by UPLC (Method 1, EXAMPLE 6) revealed the presence of side-product G-1 (5% a/a).

The same reaction was then repeated at 0° C. using an amount of methyl chloroformate that provided a conversion of no more than 93% (1.33 eq). No side-product G-1 was detected.

### Example 2: Preparation of methyl ((S)-1-(((S)-2-(4-nitrophenyl)-1-(2-(thiophen-2-yl)thiazol-4-yl)ethyl)amino)-1-oxo-3-phenylpropan-2-yl)carbamate (F-3)

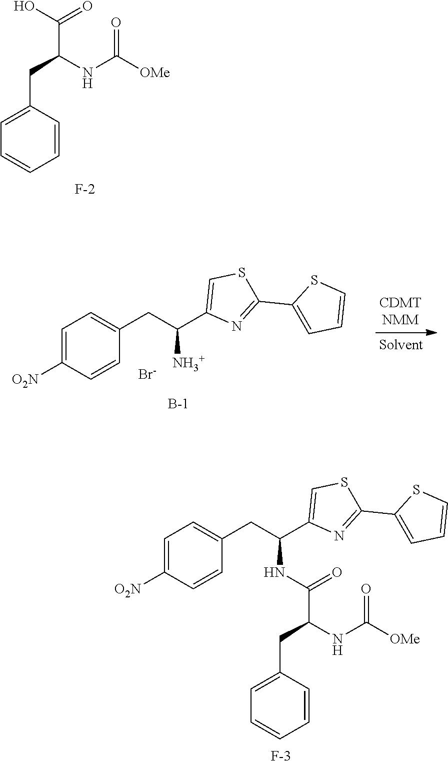

### Reaction in 2-MeTHF.

A reaction vessel is charged with B-1 (17 kg) and 2-chloro-4,6-dimethoxy-1,3,5-triazine (8 kg), and then purged with N₂ gas. 2-Methyltetrahydrofuran (236.3 kg) and 41 kg of the solution of F-2 in MTBE (24.1% w/w F-2) obtained from the previous step described in EXAMPLE 1 are added to a reaction vessel, and the contents are then warmed to 30° C. while stirring. N-methylmorpholine (9.8 kg) is then slowly added over 2 hours. The temperature of the reaction mixture is maintained below 35° C. throughout the addition. After completion of the addition, 2-methyltetrahydrofuran (8 kg) is used to rinse any remaining N-methylmorpholine from the addition line into the reaction vessel. The reaction mixture is stirred for 8 hours at 30° C. Water (84.5 kg) is then added, and the resulting mixture is heated to 40° C. The reaction mixture is allowed to stand for 30 minutes, stirred for 30 minutes, then allowed to stand for another 30 minutes. The bottom aqueous layer is drained from the reaction vessel, and the washing is repeated two additional times in a similar manner.

The remaining organic phase is then concentrated via distillation (approximately 50±5° C. at 300-400 mmHg) to 129 liters, and the resulting concentrate is then diluted with 139.4 kg of 2-methyltetrahydrofuran. The diluted mixture is then concentrated via distillation (approximately 50±5° C. at 300-400 mmHg) to 189 liters, diluted again with 139.4 kg of 2-methyltetrahydrofuran, and concentrated a final time to 196 liters via distillation (60° C. at 400 mmHg). The concentrated reaction mixture is cooled to approximately 25° C., MTBE (171.6 kg) is added, and the resulting suspension is stirred for 30 minutes at 25° C. The suspension is then filtered, the retentate is washed with 80.2 kg of MTBE, and the retentate is washed again with 79.9 kg of MTBE. The retentate is then dried under vacuum (45° C. at 10 mmHg) for 20 hours to afford F-3 (10.4 kg) as a white solid.

### Process Stream Analysis.

2-Chloro-4,6-dimethoxy-1,3,5-triazine (CDMT) (38.8 g), B-1 (85 g) and 2-MeTHF (1102 g) were added to a reaction vessel at room temperature. A solution of F-2 MTBE (37.22% w/w, 133.2 g) was rinsed into to the reactor with 2-MeTHF (200 mL) under a nitrogen atmosphere and moderate agitation. The temperature was adjusted to 22° C., and N-methylmorpholine (NMM) (47 g) was added slowly over 3 h 45 min such that the temperature was maintained between 20 and 30° C. The reaction mixture was stirred for a further 75 mins at 22° C., whereupon a sample of reaction mixture was analyzed via UPLC assay. B-1 was not detectable, and F-2 was present at 11.4 area % relative to F-3. The reaction mixture was then stirred at 22° C. for 18 hours (overnight), and again analyzed via UPLC assay. B-1 was still not detectable, and F-2 was present at 4.7 area % relative to F-3. Distilled water (856 g) was then added to the mixture. The mixture was stirred for 2 h at high speed under nitrogen, after which time the aqueous layer was drained slowly. The pH of the aqueous layer was 5-6. Distilled water (856 g) was added to the organic layer. The mixture was stirred for 30 minutes at moderate speed under nitrogen. The aqueous layer was drained slowly. The pH of the aqueous layer was 5-6. MTBE (856 g) was then added to the organic layer. After 30 minutes stirring under nitrogen at room temperature, the suspension was filtered, and the cake was rinsed with MTBE (408 g). The wet cake was further dried under vacuum at 45° C. for 10 h to afford F-3 as an off-white solid (86% yield, UPLC purity: 100%, (Method 2, EXAMPLE 6)).

The process streams were analyzed by UPLC (Method 2, EXAMPLE 6). The first and second aqueous washes removed N-methylmorpholine salts and HDMT, which were not detected in the subsequent MTBE wash and MTBE mother liquor. Less than 0.1% (w/w) F-3 was lost in the aqueous washes, and less than 6% (w/w) F-3 was lost in the MTBE mother liquor and MTBE rinse. The aqueous washes and MTBE rinse lowered unreacted levels of F-2 from 130.4% (a/a) at reaction completion to 0.1% (a/a) in the final isolated F-3.

### Reaction in THF.

B-1 (121.2 g) and THE (1573 mL) were combined in a 3-neck 5 L round-bottom flask equipped with a mechanical stirrer, agitated at room temperature for 10-30 minutes, and filtered. The filtrate was then combined with 2-chloro-4,6-dimethoxy-1,3,5-triazine (50.23 g) and THE (242 mL) in a second 3-neck, 5-L round bottom flask, and 236.63 g of a solution of F-2 in MTBE (27% w/w) was added. The resulting mixture was stirred at 20-25° C. to afford a clear, pale yellow solution, and N-methylmorpholine (NMM, 11 g, 0.4 eq) was added at a controlled rate such that the temperature of the reaction solution did not exceed 30° C. NMM-Br was observed to precipitate as a thin slurry during the addition of NMM. The mixture was stirred for 50-70 mins at 20-30° C., and a second portion of NMM was added (11 g, 0.4 eq) at 20-30° C. The mixture was further stirred for 50-70 minutes at 20-30° C., after which time a third portion of NMM (5 g, 0.2 eq) was added at 20-30° C., and the mixture was again stirred for 50-70 minutes at 20-30° C. A final portion of NMM (32 g, 1.2 eq) was then added, and the mixture was stirred for 90-120 minutes at 20-30° C., stirred at 20-25° C. for 4 to 6 hours, and stirred at 0-5° C. for 2 to 4 hours. The resulting thick white slurry was filtered, and the filter cake was washed with MTBE (2×242 mL), MeOH (2×242 mL) and another portion of MTBE (2×242 mL) to afford 292 g of a damp, off-white solid.

The off-white solid was then combined with DI water (1210 mL) in a 3-neck, 5-L round bottom flask and stirred. THE (605 mL) was added to afford a thick white slurry. The slurry was stirred at 20-25° C. for 4 to 6 hours, and then filtered. The filter cake was washed with 2:1 water/THF (v/v, 2×242 mL, 20-25° C.), and then washed with MTBE (2×242 mL, 20-25° C.) to afford 160 g of F-3 as a damp, off-white solid.

### Example 3: Preparation of Intermediate F-4

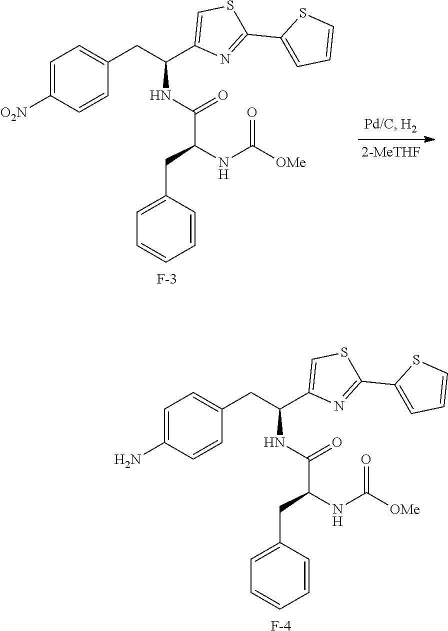

F-3 (16 kg) and 4.9% w/w palladium on carbon (3.7 kg, 56.2% H₂O) were added to a reaction vessel, which was then pressurized to approximately 40 psi with N₂ and then depressurized. The reactor was pressurized and depressurized in a similar manner twice more, and then 2-methyltetrahydrofuran (220.8 kg) was added. The mixture was heated to 45° C. and maintained at that temperature for approximately 30 minutes. The reactor was then pressurized to 30 psi with hydrogen and depressurized while maintaining a temperature of 45±5° C. The reactor was pressurized and depressurized two more times in a similar manner before the reactor was finally pressurized to 34 to 36 psi with hydrogen. Additional hydrogen gas was added to the vessel as needed to maintain the pressure between 34 psi and 36 psi throughout a 12 hour period while maintaining a temperature of 45° C. The vessel was then depressurized, purged with nitrogen gas, and a sample was withdrawn for HPLC analysis, which indicated that 0.1% of the starting material remained in the mixture. The reaction mixture was filtered to remove the palladium on carbon, and the retentate was rinsed with 2-methyltetrahydrofuran (38.4 kg). The combined filtrates were then concentrated to 127 liters via distillation at 75° C. at ambient pressure, ensuring that the distilland did not reach a temperature above 80° C. The concentrate was allowed to cool to below 60° C., and the concentrate was diluted with 89.6 kg of MTBE while maintaining a temperature of at least 48° C. The mixture was then stirred at 55° C. for 1 hour, cooled to 10° C. over 2.5 hours, and stirred for a further 30 minutes at 10° C. The resulting suspension was then filtered, and the retentate was washed with a first portion of MTBE (55.9 kg) chilled to 5° C., and then washed again with a second portion of MTBE (35.3 kg) chilled to 5° C. The washed retentate was then dried under vacuum for 22 hours (45° C. at 8-10 mmHg) to obtain F-4 (12.6 kg, 84% yield) as a white powder. UPLC: 100.00% Compound F-4 (Method 3, EXAMPLE 6).

### Example 4: Preparation of Compound 1

### Preparation of F-5 (trimethylammonium (4-((S)-2-((S)-2-((methoxycarbonyl)amino)-3-phenylpropanamido)-2-(2-(thiophen-2-yl)thiazol-4-yl)ethyl)phenyl)sulfamate)

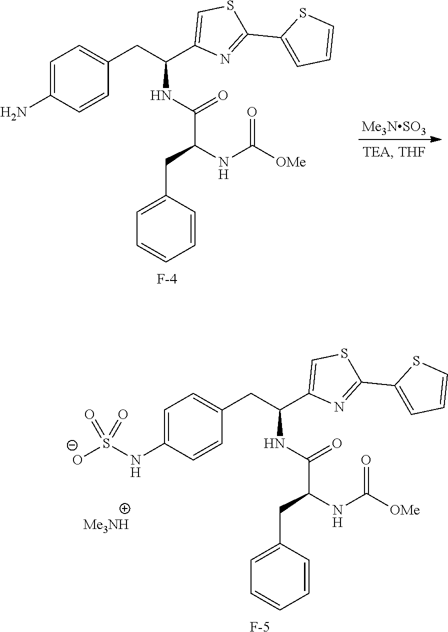

F-4 (12.4 kg), sulfur trioxide trimethylamine complex (5.7 kg), and tetrahydrofuran (65.8 kg) were added to a reaction vessel purged with nitrogen. The resulting mixture was stirred at 22° C. for 10 minutes, and triethylamine (0.2 kg) was then added. The addition line was washed with tetrahydrofuran (9 kg) to flush any remaining reagent into the reaction vessel. The resulting mixture was heated to 37° C. and stirred for 5 hours. A sample was then withdrawn for UPLC analysis (Method 4, EXAMPLE 6), which indicated that 1% of F-4 remained in the reaction mixture. The reaction mixture was cooled to 22° C. and stirred for 2 hours, and the contents were filtered and drained into another vessel using a tetrahydrofuran rinse (20.5 kg) to facilitate the transfer. MTBE (97.2 kg) was added to a separate reaction vessel and cooled to 15° C., and the tetrahydrofuran solution was added slowly over 2.5 hours while stirring. Upon completion of the addition, tetrahydrofuran (4.8 kg) was used to rinse any remaining solution from the addition line into the reaction vessel. The mixture was then stirred at 10° C. for 2 hours, and the resulting suspension was filtered at 10° C. The retentate was washed with a first portion of MTBE (30.4 kg), washed with a second portion of MTBE (31.9 kg), and then washed with a third portion of MTBE (10.6 kg). The retentate was dried under vacuum (30° C. at 10 mmHg) for 25 hours to afford F-5 (15.5 kg) as an off-white solid.

### Preparation of sodium (4-((S)-2-((S)-2-((methoxycarbonyl)amino)-3-phenylpropanamido)-2-(2-(thiophen-2-yl)thiazol-4-yl)ethyl)phenyl)sulfamate (Compound 1)

Reaction with MTBE Workup.

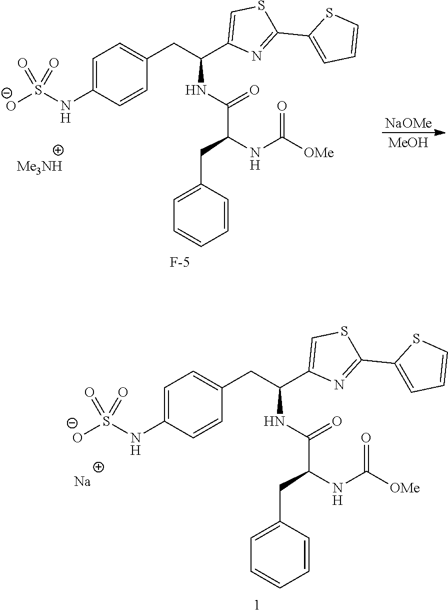

Sodium methoxide (NaOMe) (119.3 g) and methanol (137 kg) were added to a reaction vessel purged with nitrogen and stirred for 20 minutes at 22° C., and then half of the solution was transferred to a separate nitrogen-purged reaction vessel containing Compound F-5 (15.5 kg). The mixture was stirred for 20 minutes at 22° C., whereafter the remainder of the methanolic solution of NaOMe was added, and the resulting mixture was stirred for 1.5 hours at 22° C. Another portion of NaOMe (1.6 kg) was added to a separate nitrogen-purged vessel and diluted in methanol (53 kg) to provide a second methanolic NaOMe solution. The temperature of the second methanolic solution was then adjusted to 21° C. and slowly added to the solution containing Compound F-5 over 30 minutes. Upon completion of the addition, the mixture was stirred for 1 hour. The mixture was then filtered through an activated carbon filter cartridge, and the filter pad was rinsed with methanol (78 kg) before the filter pad could dry. The filtrates were combined in a reaction vessel using methanol (10 kg) to rinse any remaining filtrate into the vessel. The combined filtrates were then concentrated to 115 liters under vacuum (20° C. at 100 mmHg), and then MTBE (127.4 kg) was slowly added at 23° C. over 30 minutes. The resulting suspension was filtered, and the retentate was washed MTBE (48 kg). After stirring for 15 minutes, the washed retentate was dried under full vacuum at 35° C. for 140 hours to provide Compound 1 (11.9 kg, 81% yield from F-4) as a white to beige/tan solid. UPLC: 99% (a/a) Compound 1. Compound F-4 was below the limit of quantitation (Method 4, EXAMPLE 6).

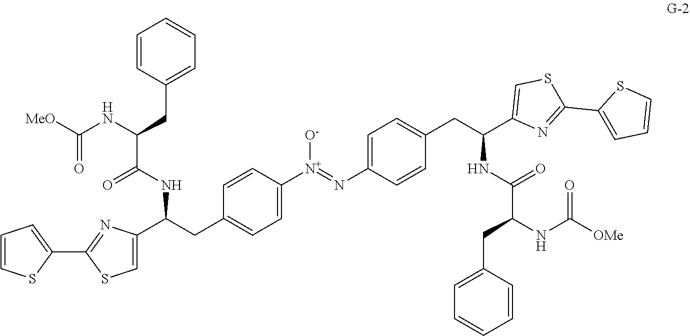 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

The isolated solid was also analyzed according to Method 5, EXAMPLE 6, and Compound G-2 was below the limit of detection (<0.8 ppm).

Reaction without Active Carbon Filtration.

25% (w/w) sodium methoxide in methanol (0.6017 kg) was diluted with methanol (328.6 kg) and stirred for 14 minutes under nitrogen and adjusted to a temperature of 22° C. A portion of the diluted sodium methoxide solution (7.0436 kg) was then added under nitrogen to a vessel containing Compound F-5 (20.7 kg) over a period of 4 minutes to afford a white precipitate. The resulting solution was then stirred for 40 minutes at 22° C., and a further portion of the diluted sodium methoxide solution (0.908 kg) was added over a period of 3 minutes. The resulting solution was then stirred for 30 minutes at 22° C., and another portion of the diluted sodium methoxide solution (0.4558 kg) was added over a period of 3 minutes. The resulting mixture was stirred for 30 minutes at 22° C. The mixture is then filtered under inert atmosphere, washing the reaction vessel and filter pad with 16.4 kg methanol. The combined filtrate and wash solution were filtered again, washing with 16.2 kg of methanol. The combined filtrate and washes were then cooled to 10° C., and the filtrate was concentrated under vacuum at a jacket temperature of 30-40° C. to a volume of 170 L. The concentrated filtrate was cooled to 22° C., and MTBE (161.4 kg) was added via an additional funnel over a period of 43 minutes to afford a thick white slurry. The slurry was stirred at 22° C. for 17 minutes and then filtered under nitrogen, washing with 61.5 kg MTBE. Vacuum was applied to the filter cake through the filter receiver, and the vacuum was counterbalanced with nitrogen flow. The vacuum was broken and the filter cake was agitated every 1 to 2 hours for 27 hours to afford a free flowing solid. Residual solvent content was further reduced by again applying vacuum to the filter receiver, counterbalancing the vacuum for 24 hours with nitrogen sparged through water, and then counterbalancing the vacuum with dry nitrogen for 22 hours to afford Compound 1 as a white solid (16.4 kg, 91% yield, UPLC: 99.38% (a/a)). 165 ppm of Compound G-2 was present in the isolated solid (UPLC Method 5, EXAMPLE 6).

Reaction with Alcohol Workup (from F-4).

A 50 L reactor, under a nitrogen atmosphere, was charged with Compound F-4 (1.3111 kg), N-methylmorpholine (0.4007 kg) and THE (6.0207 kg). To the resulting slurry was added sulfur trioxide trimethylamine complex (0.3944 kg). The reaction was heated to 55° C. and stirred for four hours, then cooled to 22° C. and stirred for 14 hours, at which point the reaction was judged to be complete by HPLC (Compound F-4<2%). The reactor was drained and rinsed with THE (1.3237 kg), and the rinse was added to the reaction mixture. The reaction mixture was polish filtered back into the reactor, the hold vessel was rinsed with THF (1.3210 kg), and the rinse was polish filtered into the reactor. The reaction mixture was concentrated under reduced pressure while maintaining the internal temperature below 35° C. The second HPLC injection showed that 3% starting material still remained.

In order to drive the reaction to completion, the distillation was stopped and the amount of THF removed was determined (2.83 kg). Fresh THE (2.8264 kg), N-methylmorpholine (0.012 kg) and sulfur trioxide trimethylamine complex (0.0118 kg) were charged. The reaction was reheated to 55° C. and stirred for four hours, then cooled to 22° C. and stirred for 16 hours, at which point the reaction was determined to be complete by HPLC (Compound F-4<2%). The reactor was rinsed with THF (1.3086 kg), and the rinse was added to the reaction mixture. The reaction mixture was polish filtered back into the reactor, and the hold vessel was rinsed with THE (1.3038 kg) and the rinse was polish filtered into the reactor. The reaction mixture was concentrated under reduced pressure to 7 L while maintaining the internal temperature below 35° C. A solution of aqueous sodium hydroxide (50% w/w, 0.2149 kg) in dehydrated alcohol (1.0491 kg) was polish filtered into the reaction vessel. The reaction mixture was concentrated under reduced pressure to 5 L while maintaining the internal temperature below 35° C. Dehydrated alcohol (5.2438 kg) was polish filtered into the reactor and the reaction mixture was vacuum distilled to approximately 8 L while maintaining the internal temperature below 35° C. Dehydrated alcohol (5.2482 kg) was polish filtered into the reactor, and the reaction mixture was vacuum distilled to approximately 12 L while maintaining the internal temperature below 35° C. Dehydrated alcohol (5.2469 kg) was polish filtered into the reactor. The temperature was adjusted to 20° C. and aged for 2 hours, and the solids were collected by vacuum filtration. The reactor and solids were washed with dehydrated alcohol (1.3088 kg) with the aid of a rubber dam. The solids were vacuum dried without heat for 73 hours, and then at 49° C. for 18 days at which point gas chromatography assay indicated that the amount of residual ethanol had plateaued at 24,046 ppm. The dried material was passed through a 300 μm sieve to afford Compound 1 (1.3367 kg, 85% yield, 97% pure by HPLC, 1.513 a/a % Compound F-4).

### Example 5: Solubility of Compounds Various Solvents

Selected intermediates of EXAMPLES 1-4 and Compound 1 were dissolved at varying concentrations in various solvents, and qualitative or quantitative solubilities were recorded at selected temperatures. The results are summarized in TABLE 1.

TABLE 1

| Compound | Solvent | Observation* |
|---|---|---|
| B-1 | THF | Soluble at 69 mg/mL |
| | 2-MeTHF | Not soluble at 69 mg/mL |
| | | S.C.~50 mg/mL at 55-60° C. |
| F-3 | THF | S.C.~5 mg/mL at 17-22° C. |
| | | S.C.~100 mg/mL at 50-55° C. |
| | 2-MeTHF | S.C.~15 mg/mL at 60-65° C. |
| | DMF | Soluble at 100 mg/mL |
| | Toluene | S.C. <5 mg/mL |
| | AcOH | |
| | AcOH, 20% aq. | |
| | MeCN | |
| | i-PrOAc | |
| F-4 | THF | S.C.~200 mg/mL |
| | Acetone | S.C.~40 mg/mL |
| | MeOH | S.C.~50 mg/mL |
| F-5 | MeOH | Soluble at 93 mg/mL |
| 1 | THF | Not soluble at 40 mg/mL |
| | Acetone | Not soluble at 60 mg/mL |
| | Acetone:H₂O (80:20) | Soluble at 53 mg/mL |
| | MeOH | S.C.~30 mg/mL |
| | MeOH:H₂O (80:20) | |
| | THF:H₂O (86:14) | |
| | DMSO | Soluble at 106 mg/mL |

*S.C. = saturation concentration. Unless otherwise noted, observations were conducted at 17-22° C.

### Example 6: Analytical Methods

### Method 1.

The content of the reaction mixtures and products of EXAMPLE 1 were evaluated using the following analytical method.
HPLC Detector: UV Visible or Diode Array Detector
UPLC Pump: UPLC Pump capable of 8000 psi or 600 bar or higher
Wavelength: 210 nm
Column: ACQUITY® UPLC HSS-C18 SB C-18, 1.8 μm, 2.1×50 mm
Mobile Phase A: 0.02% formic acid in water
Mobile Phase B: 0.02% formic acid in MeCN
Diluent: 50:50, acetonitrile:water
Mobile Phase gradient is detailed in TABLE 2:

TABLE 2

| Time | % A | % B |
|---|---|---|
| 0.0 | 99 | 1 |
| 0.5 | 99 | 1 |
| 2.5 | 50 | 50 |
| 3.5 | 15 | 85 |
| 7.0 | 15 | 85 |
| 7.1 | 99 | 1 |
| 10.0 | 99 | 1 |

Column Temperature: 20° C.
Sample Temperature: 20° C.
Flow Rate: 0.3 mL/min
Sampling rate: 5 points/sec
Injection Volume: 0.5 μL
Run Time: 10 min
Retention times are detailed in TABLE 3:

TABLE 3

| Compound | RT (min) | Approx. RRT |
|---|---|---|
| L-phenylalanine | 1.78 | 0.50 |
| F-2 | 3.60 | 1.00 |
| G-1 | 3.80 | 1.10 |

### Method 2.

The content of the reaction mixtures and products of EXAMPLE 2 were evaluated using the following analytical method.
HPLC Detector: UV Visible or Diode Array Detector
UPLC Pump: UPLC Pump capable of 8000 psi or 600 bar or higher
Wavelength: 200 nm
Pre-Column: Waters VanGuard® BEH C18, 1.7 μm, 2.1 mm×5 mm
Column: ACQUITY® UPLC BEH-C18 1.7 μm, 2.1×50 mm
Mobile Phase A: 0.02% formic acid in water
Mobile Phase B: 0.02% formic acid in MeCN
Mobile Phase gradient is detailed in TABLE 4:

TABLE 4

| Time | % A | % B |
|---|---|---|
| 0.0 | 99 | 1 |
| 4 | 15 | 85 |
| 7 | 15 | 85 |
| 7.1 | 99 | 1 |
| 10 | 99 | 1 |

Column Temperature: 40° C.
Sample Temperature: 20° C.
Flow Rate: 0.5 mL/min
Sampling rate: 5 points/sec
Injection Volume: 2 μL
Run Time: 10 min
Diluent: 100% MeOH
Retention times are detailed in TABLE 5:

TABLE 5

| Compound | RT (min) | Approx. RRT |
|---|---|---|
| HDMT* | 1.02 | 0.29 |
| CDMT** | 1.99 | 0.56 |
| F-2 | 2.05 | 0.57 |
| B-1 | 2.31 | 0.64 |
| F-3 | 3.58 | 1.00 |

*HDMT: 4,6,-dimethoxy-1,3,5-triazin-2-ol
**CDMT: 2-Chloro-4,6-dimethoxy-1,3,5-triazine

### Method 3.

The content of the reaction mixtures and products of EXAMPLE 3 were evaluated using the following analytical method.
HPLC Detector: UV Visible or Diode Array Detector
UPLC Pump: UPLC Pump capable of 8000 psi or 600 bar or higher
Wavelength: 318 nm
Pre-Column: Waters VanGuard® BEH C18, 1.7 μm, 2.1 mm×5 mm
Column: ACQUITY-UPLC-BEH-C18 1.7 μm, 2.1×50 mm
Mobile Phase A: 0.05% trifluoroacetic acid in water
Mobile Phase B: 0.05% trifluoroacetic acid in acetonitrile
Mobile Phase gradient is detailed in TABLE 6:

TABLE 6

| Time | % A | % B |
|---|---|---|
| 0.0 | 70 | 30 |
| 7 | 0 | 100 |
| 9 | 0 | 100 |
| 9.1 | 70 | 30 |
| 12 | 70 | 30 |

Column Temperature: 45° C.
Sample Temperature: 20° C.
Flow Rate: 0.5 mL/min
Injection Volume: 1 μL
Run Time: 12 min
Diluent: 100% MeOH
Retention times are detailed in TABLE 7:

TABLE 7

| Compound | RT (min) | Approx. RRT |
|---|---|---|
| F-4 | 1.58 | 1.00 |
| F-3 | 3.40 | 2.15 |

### Method 4.

The content of the reaction mixtures and products of EXAMPLE 4 were evaluated using the following analytical method.
HPLC Detector: UV Visible or Diode Array Detector
UPLC Pump: UPLC Pump capable of 8000 psi or 600 bar or higher
Wavelength: 318 nm
Column: ACQUITY® UPLC BEH-C18 1.7 μm, 2.1×50 mm
Mobile Phase A: 90:10-0.02 M Phosphate Buffer pH=8.0: MeOH
Mobile Phase B: 100% MeOH
Mobile Phase gradient is detailed in TABLE 8:

TABLE 8

| Time | % A | % B |
|---|---|---|
| 0.0 | 70 | 30 |
| 1 | 70 | 30 |
| 7 | 0 | 100 |
| 9 | 0 | 100 |
| 9.1 | 70 | 30 |
| 12 | 70 | 30 |

Column Temperature: 60° C.
Sample Temperature: 20° C.
Flow Rate: 0.5 mL/min
Injection Volume: 2 μL
Run Time: 12 min
Diluent: 50:50-0.02 M Phosphate Buffer pH=8.0: MeOH
Retention times are detailed in TABLE 9:

TABLE 9

| Compound | RT (min) | Approx. RRT |
|---|---|---|
| 1 | 2.92 | 0.91 |
| F-5 | 3.32 | 1.00 |
| F-4 | 4.08 | 1.26 |

### Method 5.

The content of the product of EXAMPLE 4 was evaluated using the following analytical method.
HPLC Detector: UV Visible or Diode Array Detector
UPLC Pump: UPLC Pump capable of 8000 psi or 600 bar or higher
Wavelength: 322 nm
Column: Phenomenex Kinetex® C18 2.6 μm, 4.6×150 mm
Mobile Phase A: 2:8 [1% AcOH in 50 mM aq. NH₄OAc]:[0.1% triethylamine in acetonitrile]
Mobile Phase B: 8:2 [1% AcOH in 50 mM aq. NH₄OAc]:[0.1% triethylamine in acetonitrile]
Mobile Phase gradient is detailed in TABLE 10:

TABLE 10

| Time | % A | % B |
|---|---|---|
| 0.0 | 20 | 80 |
| 6 | 100 | 0 |
| 15 | 100 | 0 |
| 16 | 20 | 80 |
| 20 | 20 | 80 |

Column Temperature: 45° C.
Sample Temperature: 25° C.
Flow Rate: 0.7 mL/min
Injection Volume: 5 μL
Run Time: 20 min
Diluent: 9:1 MeOH:DMSO
Retention times are detailed in TABLE 11:

TABLE 11

| Compound | RT (min) |
|---|---|
| 1 | 4.5 |
| G-2 | 9.9 |

A relative response factor of 0.52 for Compound G-2 with respect to Compound 1 was determined from the following linearity curves: Cpd G-2 (mg/mL)=([Signal area]-237.605)/17088312.948; Cpd 1 (mg/mL)=([Signal area]-350.964)/8910968.340.

### Embodiments

Embodiment A1. A pharmaceutical composition comprising a mixture of a Tie-2 modulator and a second compound, wherein:
(a) each of the Tie-2 modulator and the second compound has a core structure and a nitrogen atom substituent bound to the core structure at a position on the core structure;
(b) the core structure of the Tie-2 modulator is identical to the core structure of the second compound;
(c) the position on the core structure of the Tie-2 modulator to which the nitrogen atom substituent is bound is identical to the position on the core structure of the second compound to which the nitrogen atom substituent is bound;
(d) the nitrogen atom substituent of the Tie-2 modulator is —N(H)(E), wherein E is a group that contains a sulfur atom bound to the nitrogen atom;
(e) the nitrogen atom substituent of the second compound is —NH₂; and
(f) the pharmaceutical composition is substantially free of solvent.

Embodiment A2. The pharmaceutical composition of embodiment A1, wherein the Tie-2 modulator forms at least about 99.0% (a/a) of the mixture as determined by a liquid chromatography assay, and wherein the second compound forms from about 0.001% to about 0.5% (a/a) of the mixture as determined by the liquid chromatography assay.

Embodiment A3. The pharmaceutical composition of embodiment A1 or embodiment A2, wherein the nitrogen atom substituent of the Tie-2 modulator is a sulfamate group.

Embodiment A4. The pharmaceutical composition of any one of embodiments A1-A3, wherein the second compound is a desulfonylation congener of the Tie-2 modulator.

Embodiment A5. The pharmaceutical composition of any one of embodiments A1-A4, wherein:
the Tie-2 modulator has a structure of Q-Z; and
the second compound has a structure of W—Z, wherein
Q is

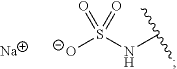

W is H₂N—; and
each Z is the core structure.

Embodiment A6. The pharmaceutical composition of embodiment A5, wherein each Z is.

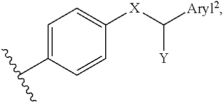

wherein
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; and
Y is H, aryl, heteroaryl, NH(aryl), NH(heteroaryl), or NHCOR^{g}, any of which is substituted or unsubstituted, or

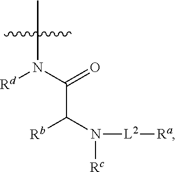

wherein:
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted.

Embodiment A7. The pharmaceutical composition of embodiment A6, wherein:
Aryl² is substituted or unsubstituted heteroaryl; and
X is alkylene.

Embodiment A8. The pharmaceutical composition of embodiment A6 or embodiment A7, wherein:
Aryl² is substituted heteroaryl; and
X is methylene.

Embodiment A9. The pharmaceutical composition of any one of embodiments A5-A8, wherein each Z is:

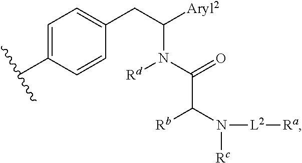

wherein:
Aryl² is substituted heteroaryl;
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

Embodiment A10. The pharmaceutical composition of any one of embodiments A6-A9, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

Embodiment A11. The pharmaceutical composition of any one of embodiments A6-A10, wherein Aryl² is:

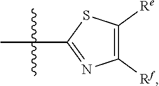

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment A12. The pharmaceutical composition of embodiment A11, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment A13. The pharmaceutical composition of embodiment A11, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment A14. The pharmaceutical composition of any one of embodiments A11-A13, wherein:
R^{a} is alkyl, which is substituted or unsubstituted;
R^{b} is arylalkyl, which is substituted or unsubstituted;
R^{e} is H; and
R^{f} is heteroaryl.

Embodiment A15. The pharmaceutical composition of any one of embodiments A5-A14, wherein each Z is:

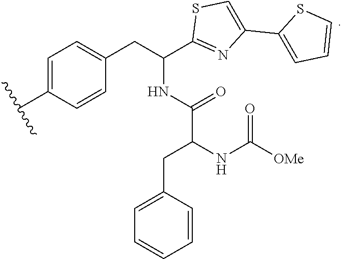 [SMILES: *c1ccc(CC(NC(=O)C(Cc2ccccc2)NC(=O)OC)c2nc(-c3cccs3)cs2)cc1]

Embodiment A16. The pharmaceutical composition of any one of embodiments A5-A15, wherein each Z is:

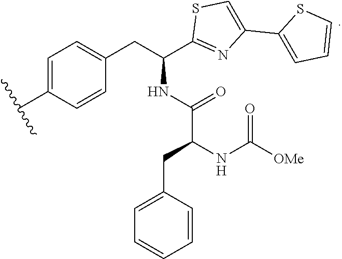 [SMILES: *c1ccc(C[C@H](NC(=O)[C@H](Cc2ccccc2)NC(=O)OC)c2nc(-c3cccs3)cs2)cc1]

Embodiment A17. The pharmaceutical composition of any one of embodiments A6-A10, wherein Aryl² is:

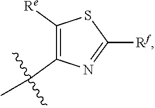

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment A18. The pharmaceutical composition of embodiment A17, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment A19. The pharmaceutical composition of embodiment A17, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment A20. The pharmaceutical composition of any one of embodiments A17-A19, wherein:
R^{a} is alkyl, which is substituted or unsubstituted;
R^{b} is arylalkyl, which is substituted or unsubstituted;
R^{e} is H; and
R^{f} is alkyl.

Embodiment A21. The pharmaceutical composition of any one of embodiments A5-A10 and A17-A20, wherein each Z is:

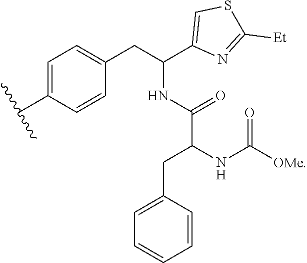 [SMILES: *c1ccc(CC(NC(=O)C(Cc2ccccc2)NC(=O)OC)c2csc(CC)n2)cc1]

Embodiment A22. The pharmaceutical composition of any one of embodiments A5-A10 and A17-A21, wherein each Z is:

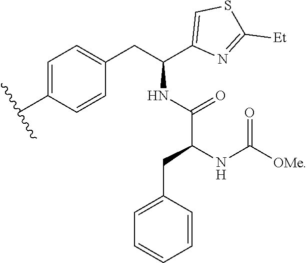 [SMILES: *c1ccc(C[C@H](NC(=O)[C@H](Cc2ccccc2)NC(=O)OC)c2csc(CC)n2)cc1]

Embodiment A23. The pharmaceutical composition of any one of embodiments A17-A19, wherein:
R^{a} is alkyl, which is substituted or unsubstituted;
R^{b} is arylalkyl, which is substituted or unsubstituted;
R^{e} is H; and
R^{f} is heteroaryl.

Embodiment A24. The pharmaceutical composition of any one of embodiments A5-A10, A17-A19, and A23, wherein each Z is:

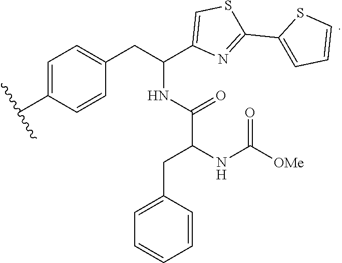 [SMILES: *c1ccc(CC(NC(=O)C(Cc2ccccc2)NC(=O)OC)c2csc(-c3cccs3)n2)cc1]

Embodiment A25. The pharmaceutical composition of any one of embodiments A5-A10, A17-A19, A23, and A24, wherein each Z is:

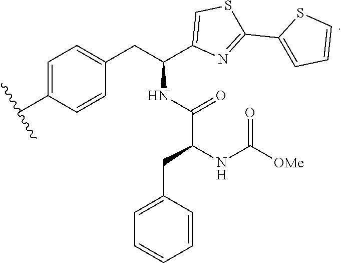 [SMILES: *c1ccc(C[C@H](NC(=O)[C@H](Cc2ccccc2)NC(=O)OC)c2csc(-c3cccs3)n2)cc1]

Embodiment A26. The pharmaceutical composition of any one of embodiments A1-A25, wherein the Tie-2 modulator forms at least about 99.2% (a/a) of the mixture as determined by the liquid chromatography assay.

Embodiment A27. The pharmaceutical composition of any one of embodiments A1-A25, wherein the Tie-2 modulator forms at least about 99.3% (a/a) of the mixture as determined by the liquid chromatography assay.

Embodiment A28. The pharmaceutical composition of any one of embodiments A1-A25, wherein the Tie-2 modulator forms at least about 99.4% (a/a) of the mixture as determined by the liquid chromatography assay.

Embodiment A29. The pharmaceutical composition of any one of embodiments A1-A25, wherein the Tie-2 modulator forms from about 99.3% to about 99.5% (a/a) of the mixture as determined by the liquid chromatography assay.

Embodiment A30. The pharmaceutical composition of any one of embodiments A1-A29, wherein the second compound forms from about 0.001% to about 0.1% (a/a) of the mixture as determined by the liquid chromatography assay.

Embodiment A31. The pharmaceutical composition of any one of embodiments A1-A29, wherein the second compound forms from about 0.01% to about 0.1% (a/a) of the mixture as determined by the liquid chromatography assay.

Embodiment A32. The pharmaceutical composition of any one of embodiments A1-A29, wherein the second compound forms from about 0.01% to about 0.03% (a/a) of the mixture as determined by the liquid chromatography assay.

Embodiment A33. The pharmaceutical composition of any one of embodiments A1-A32, wherein the Tie-2 modulator is a Tie-2 activator.

Embodiment A34. The pharmaceutical composition of any one of embodiments A1-A32, wherein the Tie-2 modulator is a HPTPβ inhibitor.

Embodiment A35. The pharmaceutical composition of any one of embodiments A1-A34, wherein the composition comprises no more than about 100 ppm of a third compound as determined by HPLC, wherein the third compound comprises an azoxy moiety.

Embodiment A36. The pharmaceutical composition of any one of embodiments A1-A34, wherein the composition comprises no more than about 10 ppm of a third compound as determined by HPLC, wherein the third compound comprises an azoxy moiety.

Embodiment A37. The pharmaceutical composition of any one of embodiments A1-A34, wherein the composition comprises no more than about 11 ppm of a third compound as determined by HPLC, wherein the third compound comprises an azoxy moiety.

Embodiment A38. The pharmaceutical composition of any one of embodiments A5-A25, wherein the composition comprises no more than about 100 ppm of a third compound as determined by HPLC, wherein the third compound has a structure of Z-J-Z, wherein J is

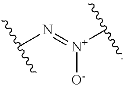

Embodiment A39. The pharmaceutical composition of any one of embodiments A5-A25, wherein the composition comprises no more than about 1 ppm of a third compound as determined by HPLC, wherein the third compound has a structure of Z-J-Z, wherein J is

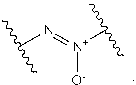 [SMILES: */N=[N+](/*)[O-]]

Embodiment B1. A process for preparing a composition, the process comprising:
(i) contacting an initial quantity of an amine with a sulfur trioxide source in a solvent to afford a first mixture, wherein the first mixture comprises a quantity of a first ion pair that is a sulfamate anion and an organic cation; and
(ii) contacting the first ion pair with a sodium cation source to provide a second mixture, wherein the second mixture comprises a second ion pair and the amine, wherein the second ion pair is a sodium cation and the sulfamate anion,
wherein the initial quantity of the amine is at least 1 kg, and a ratio of the sulfamate anion to the amine in the second mixture is at least 99:1 (a/a) as determined by a liquid chromatography assay.

Embodiment B2. The process of embodiment B1, wherein the initial quantity of amine is contacted with the sulfur trioxide source in the presence of an organic solvent.

Embodiment B3. The process of embodiment B1 or embodiment B2, wherein the initial quantity of amine is contacted with the sulfur trioxide source in the presence of tetrahydrofuran.

Embodiment B4. The process of any one of embodiments B1-B3, wherein the initial quantity of amine is contacted with the sulfur trioxide source in the presence of an amine base.

Embodiment B5. The process of any one of embodiments B1-B4, wherein the initial quantity of amine is contacted with the sulfur trioxide source in the presence of triethylamine.

Embodiment B6. The process of any one of embodiments B1-B5, further comprising filtering the second mixture through activated carbon to provide a filtrate.

Embodiment B7. The process of embodiment B6, further comprising treating the filtrate to provide a solid, wherein the solid comprises the second ion pair and the amine.

Embodiment B8. The process of embodiment B7, wherein the treating comprises adding an antisolvent to the filtrate to provide a suspension and isolating the solid from the suspension.

Embodiment B9. The process of embodiment B8, wherein the antisolvent is MTBE.

Embodiment B10. The process of any one of embodiments B1-B9, wherein the amine comprises an arylene moiety substituted with a primary amine.

Embodiment B11. The process of any one of embodiments B1-B10, wherein:
a) the sulfamate anion comprises the moiety:

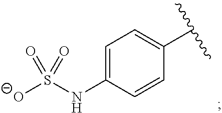 [SMILES: *c1ccc(NS(=O)(=O)[O-])cc1]

and
b) the amine comprises the moiety:

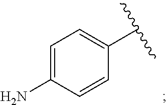 [SMILES: *c1ccc(N)cc1]

Embodiment B12. The process of any one of embodiments B1-B11, wherein:
a) the sulfamate anion is of formula (I):

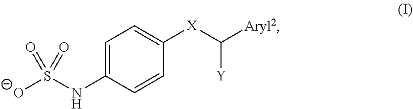

and
b) the amine is of formula (II):

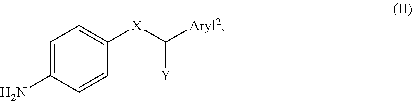

wherein
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; and
Y is H, aryl, heteroaryl, NH(aryl), NH(heteroaryl), NHSO₂R^{g}, or NHCOR^{g}, any of which is substituted or unsubstituted, or

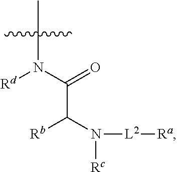

wherein:
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted.

Embodiment B13. The process of embodiment B12, wherein:
Aryl² is substituted or unsubstituted heteroaryl; and
X is alkylene.

Embodiment B14. The process of embodiment B12 or embodiment B13, wherein:
Aryl² is substituted heteroaryl; and
X is methylene.

Embodiment B15. The process of any one of embodiments B1-B14, wherein:
i) the sulfamate anion is of formula (Ia):

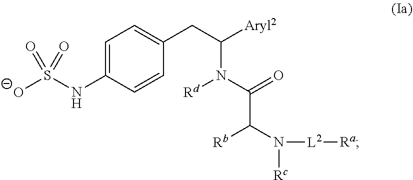

and
ii) the amine is of formula (IIa):

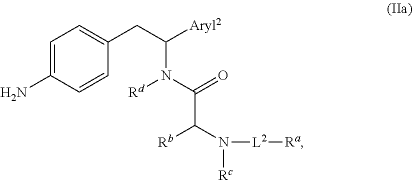

wherein:
Aryl² is substituted heteroaryl;
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

Embodiment B16. The process of any one of embodiments B12-B15, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

Embodiment B17. The process of any one of embodiments B12-B16, wherein Aryl² is:

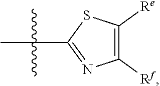

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment B18. The process of embodiment B17, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment B19. The process of embodiment B17, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment B20. The process of any one of embodiments B17-B19, wherein:
R^{a} is alkyl, which is substituted or unsubstituted;
R^{b} is arylalkyl, which is substituted or unsubstituted;
R^{e} is H; and
R^{f} is heteroaryl.

Embodiment B21. The process of any one of embodiments B1-B20, wherein:
i) the sulfamate anion is of formula (Ia1):

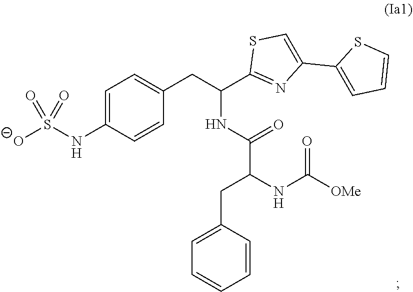 [SMILES: COC(=O)NC(Cc1ccccc1)C(=O)NC(Cc1ccc(NS(=O)(=O)[O-])cc1)c1nc(-c2cccs2)cs1]

and
ii) the amine is of formula (IIa1):

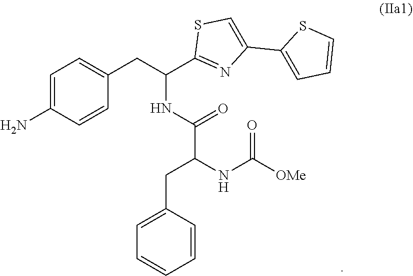 [SMILES: COC(=O)NC(Cc1ccccc1)C(=O)NC(Cc1ccc(N)cc1)c1nc(-c2cccs2)cs1]

Embodiment B22. The process of any one of embodiments B1-B20, wherein:
i) the sulfamate anion is of formula (Ia2):

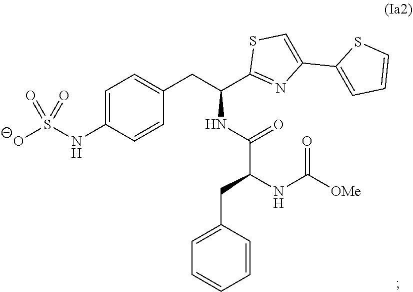 [SMILES: COC(=O)N[C@@H](Cc1ccccc1)C(=O)N[C@@H](Cc1ccc(NS(=O)(=O)[O-])cc1)c1nc(-c2cccs2)cs1]

and
ii) the amine is of formula (IIa2):

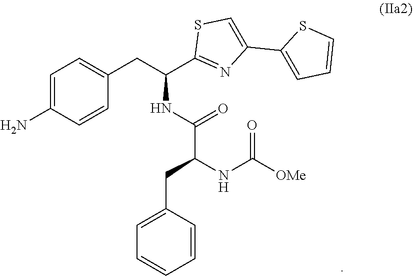 [SMILES: COC(=O)N[C@@H](Cc1ccccc1)C(=O)N[C@@H](Cc1ccc(N)cc1)c1nc(-c2cccs2)cs1]

Embodiment B23. The process of any one of embodiments B12-B16, wherein Aryl² is:

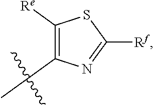

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment B24. The process of embodiment B23, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment B25. The process of embodiment B23, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment B26. The process of any one of embodiments B23-B25, wherein:
R^{a} is alkyl, which is substituted or unsubstituted;
R^{b} is arylalkyl, which is substituted or unsubstituted;
R^{e} is H; and
R^{f} is alkyl.

Embodiment B27. The process of any one of embodiments B1-B16 and B23-B26, wherein:
i) the sulfamate anion is of formula (Ia3):

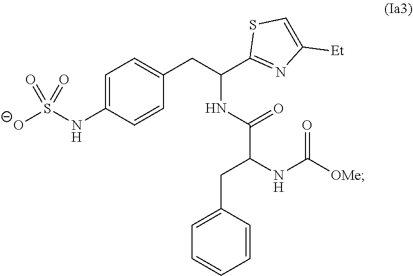 [SMILES: CCc1csc(C(Cc2ccc(NS(=O)(=O)[O-])cc2)NC(=O)C(Cc2ccccc2)NC(=O)OC)n1]

and
ii) the amine is of formula (IIa3):

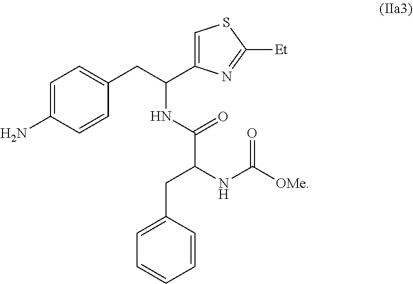 [SMILES: CCc1nc(C(Cc2ccc(N)cc2)NC(=O)C(Cc2ccccc2)NC(=O)OC)cs1]

Embodiment B28. The process of any one of embodiments B1-B16 and B23-B27, wherein:
i) the sulfamate anion is of formula (Ia4):

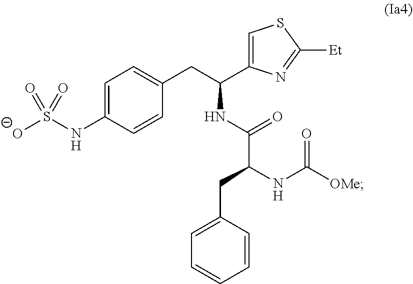 [SMILES: CCc1nc([C@H](Cc2ccc(NS(=O)(=O)[O-])cc2)NC(=O)[C@H](Cc2ccccc2)NC(=O)OC)cs1]

and
ii) the amine is of formula (IIa4):

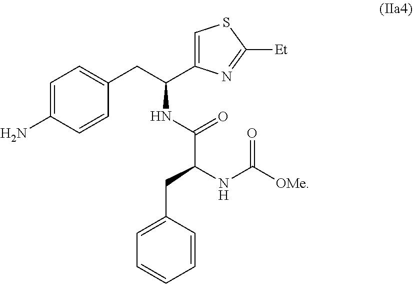 [SMILES: CCc1nc([C@H](Cc2ccc(N)cc2)NC(=O)[C@H](Cc2ccccc2)NC(=O)OC)cs1]

Embodiment B29. The process of any one of embodiments B23-B25, wherein:
R^{a} is alkyl, which is substituted or unsubstituted;
R^{b} is arylalkyl, which is substituted or unsubstituted;
R^{e} is H; and
R^{f} is heteroaryl.

Embodiment B30. The process of any one of embodiments B1-B16, B23-B25, and B29, wherein:
i) the sulfamate anion is of formula (Ia5):

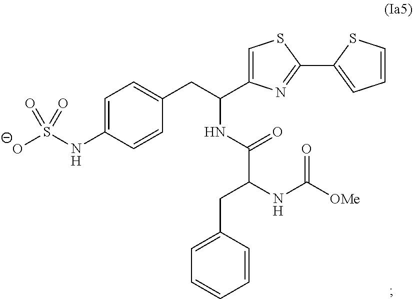 [SMILES: COC(=O)NC(Cc1ccccc1)C(=O)NC(Cc1ccc(NS(=O)(=O)[O-])cc1)c1csc(-c2cccs2)n1]

and
ii) the amine is of formula (IIa5):

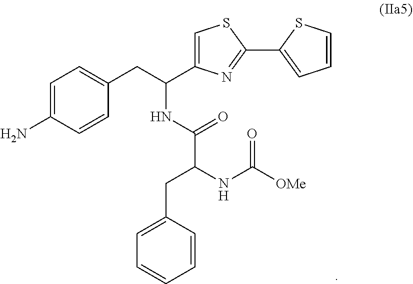 [SMILES: COC(=O)NC(Cc1ccccc1)C(=O)NC(Cc1ccc(N)cc1)c1csc(-c2cccs2)n1]

Embodiment B31. The process of any one of embodiments B1-B16, B23-B25, B29, and B30, wherein:
i) the sulfamate anion is of formula (Ia6):

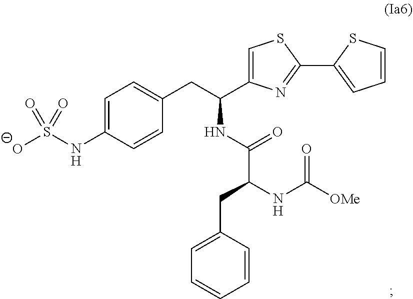 [SMILES: COC(=O)N[C@@H](Cc1ccccc1)C(=O)N[C@@H](Cc1ccc(NS(=O)(=O)[O-])cc1)c1csc(-c2cccs2)n1]

ii) the amine is of formula (IIa6):

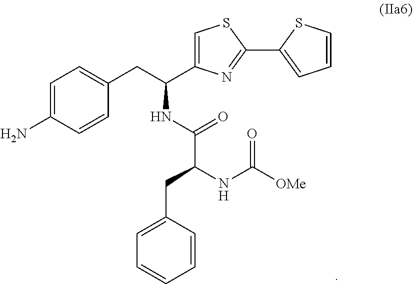 [SMILES: COC(=O)N[C@@H](Cc1ccccc1)C(=O)N[C@@H](Cc1ccc(N)cc1)c1csc(-c2cccs2)n1]

Embodiment B32. The process of any one of embodiments B1-B31, wherein the organic cation is a trialkylammonium cation.

Embodiment B33. The process of any one of embodiments B1-B32, wherein the organic cation is HNMe₃*.

Embodiment B34. The process of any one of embodiments B1-B33, wherein an area/area ratio of the sulfamate anion to the amine in the second mixture is at least 99.1:0.9 as determined by the liquid chromatography assay.

Embodiment B35. The process of any one of embodiments B1-B33, wherein an area/area ratio of the sulfamate anion to the amine in the second mixture is at least 99.2:0.8 as determined by the liquid chromatography assay.

Embodiment B36. The process of any one of embodiments B1-B33, wherein an area/area ratio of the sulfamate anion to the amine in the second mixture is at least 99.3:0.7 as determined by the liquid chromatography assay.

Embodiment B37. The process of any one of embodiments B6-B8, wherein the second ion pair forms at least about 99.2% (a/a) of the solid as determined by the liquid chromatography assay.

Embodiment B38. The process of any one of embodiments B6-B8, wherein the second ion pair forms at least about 99.3% (a/a) of the solid as determined by the liquid chromatography assay.

Embodiment B39. The process of any one of embodiments B6-B8, wherein the second ion pair forms at least about 99.4% (a/a) of the solid as determined by the liquid chromatography assay.

Embodiment B40. The process of any one of embodiments B6-B8, wherein the second ion pair forms from about 99.3% to about 99.5% (a/a) of the solid as determined by the liquid chromatography assay.

Embodiment B41. The process of any one of embodiments B6-B8 and B37-B40, wherein the amine forms about 0.001% to about 0.1% (a/a) of the solid as determined by the liquid chromatography assay.

Embodiment B42. The process of any one of embodiments B6-B8 and B37-B40, wherein the amine forms from about 0.01% to about 0.1% (a/a) of the solid as determined by the liquid chromatography assay.

Embodiment B43. The process of any one of embodiments B6-B8 and B37-B40, wherein the amine forms from about 0.01% to about 0.03% (a/a) of the solid as determined by the liquid chromatography assay.

Embodiment B44. The process of any one of embodiments B6-B8 and B37-B43, wherein the solid comprises no more than about 100 ppm of an azoxy compound.

Embodiment B45. The process of any one of embodiments B6-B8 and B37-B43, wherein the solid comprises no more than about 1 ppm of an azoxy compound.

Embodiment B46. The process of embodiment B44 and embodiment B45, wherein the azoxy compound is:

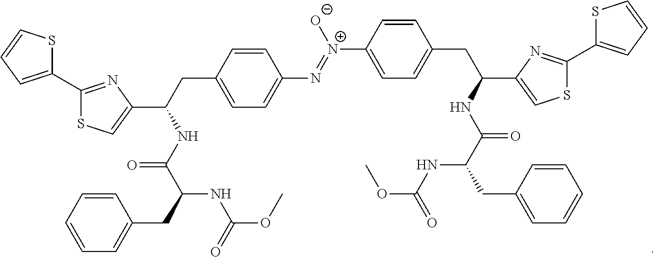 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

Embodiment B47. The process of any one of embodiments B1-B46, wherein the initial quantity of the amine is at least 10 kg.

Embodiment B48. The process of any one of embodiments B1-B46, wherein the initial quantity of the amine is from about 1 kg to about 100 kg.

Embodiment B49. The process of any one of embodiments B1-B48, wherein a chemical yield of the second ion pair is at least about 60%, with respect to the quantity of the initial quantity of the amine.

Embodiment B50. The process of any one of embodiments B1-B48, wherein a chemical yield of the second ion pair is at least about 70%, with respect to the quantity of the initial quantity of the amine.

Embodiment B51. The process of any one of embodiments B1-B48, wherein a chemical yield of the second ion pair is from about 70% to about 99%, with respect to the initial quantity of the amine.

Embodiment B52. The process of any one of embodiments B1-B48, wherein a chemical yield of the second ion pair is from about 70% to about 90%, with respect to the initial quantity of the amine.

Embodiment B53. The process of any one of embodiments B1-B46, wherein a chemical yield of the second ion pair is at least about 10 kg.

Embodiment B54. The process of any one of embodiments B1-B46, wherein a chemical yield of the second ion pair is from about 10 kg to about 100 kg.

Embodiment B55. The process of any one of embodiments B1-B46, wherein a chemical yield of the second ion pair is from about 10 kg to about 20 kg.

Embodiment B56. The process of any one of embodiments B1-B55, wherein the sulfur trioxide source is a complex of sulfur trioxide and an organic molecule that comprises a nitrogen atom.

Embodiment B57. The process of any one of embodiments B1-B56, wherein the sulfur trioxide source is a complex of sulfur trioxide and trimethylamine.

Embodiment B58. The process of any one of embodiments B1-B46, wherein a chemical yield of the second ion pair is at least about 80%, with respect to the quantity of the first ion pair.

Embodiment B59. The process of any one of embodiments B1-B46, wherein a chemical yield of the second ion pair is at least about 90%, with respect to the quantity of the first ion pair.

Embodiment B60. The process of any one of embodiments B1-B46, wherein a chemical yield of the second ion pair is at least about 95%, with respect to the quantity of the first ion pair.

Embodiment B61. The process of any one of embodiments B1-B46, wherein a chemical yield of the second ion pair is from about 95% to about 99%, with respect to the quantity of the first ion pair.

Embodiment B62. The process of any one of embodiments B1-B61, wherein the sodium cation source comprises an alkoxide salt.

Embodiment B63. The process of any one of embodiments B1-B62, wherein the sodium cation source comprises sodium methoxide.

Embodiment C1. A process comprising reducing a nitro compound in presence of a solvent to provide a reaction mixture comprising an amino compound, wherein the amino compound is a desulfonylation congener of a Tie-2 modulator, and a solubility of the solvent in water is less than about 20 grams of the solvent per 100 grams of water at 20° C.

Embodiment C2. The process of embodiment C1, wherein the nitro compound comprises a para-nitroarylene moiety.

Embodiment C3. The process of embodiment C1 or embodiment C2, wherein the nitro compound is of formula (IV):

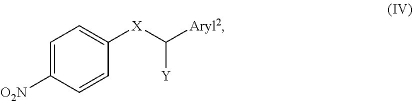

and
the amine is of formula (II):

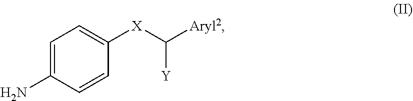

wherein
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; and
Y is H, aryl, heteroaryl, NH(aryl), NH(heteroaryl), NHSO₂R^{g}, or NHCOR^{g}, any of which is substituted or unsubstituted, or

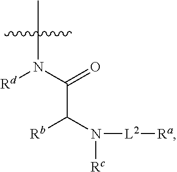

wherein:
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R_{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted.

Embodiment C4. The process of embodiment C3, wherein:
Aryl² is substituted or unsubstituted heteroaryl; and
X is alkylene.

Embodiment C5. The process of embodiment C3 or embodiment C4, wherein:
Aryl² is substituted heteroaryl; and
X is methylene.

Embodiment C6. The process of any one of embodiments C1-C5, wherein:
i) the nitro compound is of formula (IVa):

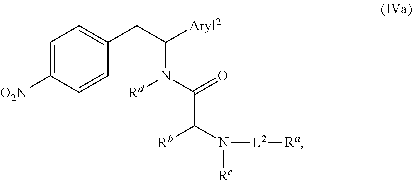

and
ii) the amine is of formula (IIa):

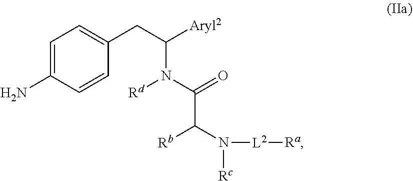

wherein:
Aryl² is substituted heteroaryl;
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

Embodiment C7. The process of any one of embodiments C3-C6, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

Embodiment C8. The process of any one of embodiments C3-C7, wherein Aryl² is:

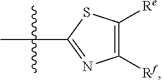

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment C9. The process of embodiment C8, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment C10. The process of embodiment C8, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment C11. The process of any one of embodiments C8-C10, wherein:
R^{a} is alkyl, which is substituted or unsubstituted;
R^{b} is arylalkyl, which is substituted or unsubstituted;
R^{e} is H; and
R^{f} is heteroaryl.

Embodiment C12. The process of any one of embodiments C1-C11, wherein:
i) the nitro compound is of formula (IVa1):

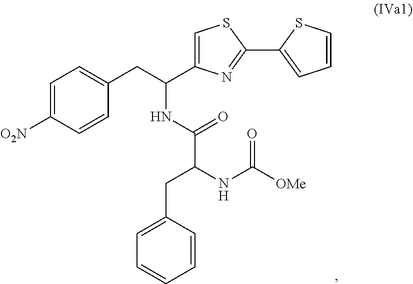 [SMILES: COC(=O)NC(Cc1ccccc1)C(=O)NC(Cc1ccc([N+](=O)[O-])cc1)c1csc(-c2cccs2)n1]

and
ii) the amine is of formula (IIa1):

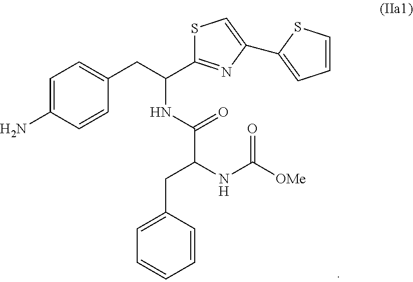 [SMILES: COC(=O)NC(Cc1ccccc1)C(=O)NC(Cc1ccc(N)cc1)c1nc(-c2cccs2)cs1]

Embodiment C13. The process of any one of embodiments C1-C12, wherein:
i) the nitro compound is of formula (Ia2):

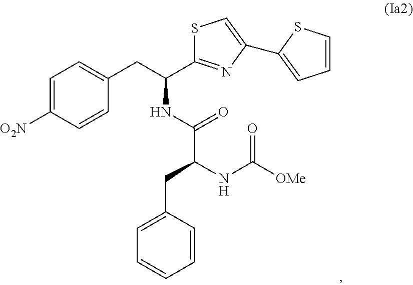 [SMILES: COC(=O)N[C@@H](Cc1ccccc1)C(=O)N[C@@H](Cc1ccc([N+](=O)[O-])cc1)c1nc(-c2cccs2)cs1]

and
ii) the amine is of formula (IIa2):

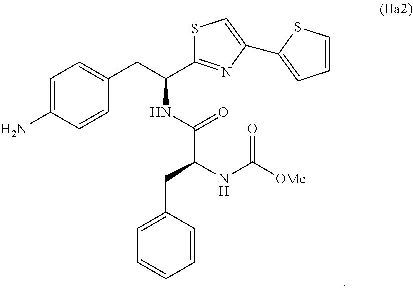 [SMILES: COC(=O)N[C@@H](Cc1ccccc1)C(=O)N[C@@H](Cc1ccc(N)cc1)c1nc(-c2cccs2)cs1]

Embodiment C14. The process of any one of embodiments C3-C7, wherein Aryl² is:

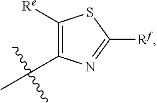

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment C15. The process of embodiment C14, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment C16. The process of embodiment C14, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment C17. The process of any one of embodiments C14-C16, wherein:
R^{a} is alkyl, which is substituted or unsubstituted;
R^{b} is arylalkyl, which is substituted or unsubstituted;
R^{e} is H; and
R^{f} is alkyl.

Embodiment C18. The process of any one of embodiments C1-C7 and C14-C17, wherein:
i) the nitro compound is of formula (IVa3):

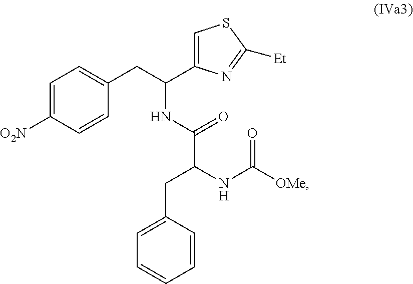 [SMILES: CCc1nc(C(Cc2ccc([N+](=O)[O-])cc2)NC(=O)C(Cc2ccccc2)NC(=O)OC)cs1]

and
ii) the amine is of formula (IIa3):

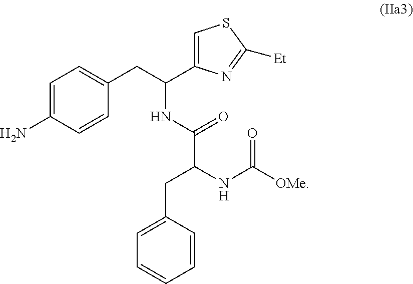 [SMILES: CCc1nc(C(Cc2ccc(N)cc2)NC(=O)C(Cc2ccccc2)NC(=O)OC)cs1]

Embodiment C19. The process of any one of embodiments C1-C7 and C14-C18, wherein:
i) the nitro compound is of formula (IVa4):

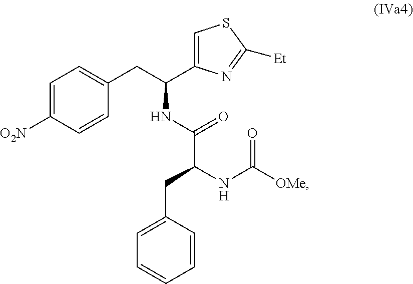 [SMILES: CCc1nc([C@H](Cc2ccc([N+](=O)[O-])cc2)NC(=O)[C@H](Cc2ccccc2)NC(=O)OC)cs1]

and
ii) the amine is of formula (IIa4):

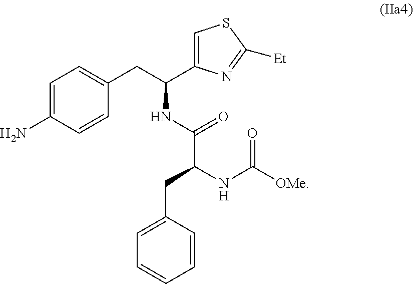 [SMILES: CCc1nc([C@H](Cc2ccc(N)cc2)NC(=O)[C@H](Cc2ccccc2)NC(=O)OC)cs1]

Embodiment C20. The process of any one of embodiments C14-C16, wherein:
R^{a} is alkyl, which is substituted or unsubstituted;
R^{b} is arylalkyl, which is substituted or unsubstituted;
R^{e} is H; and
R^{f} is heteroaryl.

Embodiment C21. The process of any one of embodiments C1-C7, C14-C16, and C20, wherein:
i) the nitro compound is of formula (IVa5):

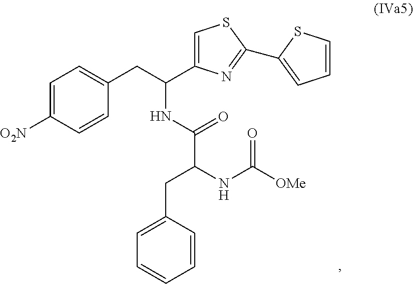 [SMILES: COC(=O)NC(Cc1ccccc1)C(=O)NC(Cc1ccc([N+](=O)[O-])cc1)c1csc(-c2cccs2)n1]

and
ii) the amine is of formula (IIa5):

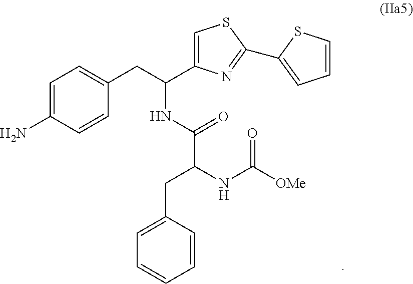 [SMILES: COC(=O)NC(Cc1ccccc1)C(=O)NC(Cc1ccc(N)cc1)c1csc(-c2cccs2)n1]

Embodiment C22. The process of any one of embodiments C1-C7, C14-C16, C20, and C21, wherein:
i) the nitro compound is of formula (IVa6):

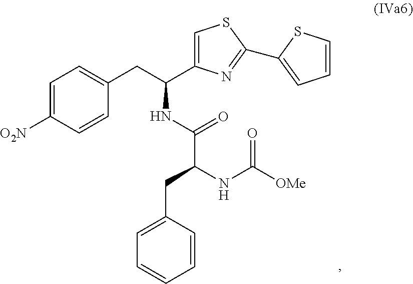 [SMILES: COC(=O)N[C@@H](Cc1ccccc1)C(=O)N[C@@H](Cc1ccc([N+](=O)[O-])cc1)c1csc(-c2cccs2)n1]

and
ii) the amine is of formula (IIa6):

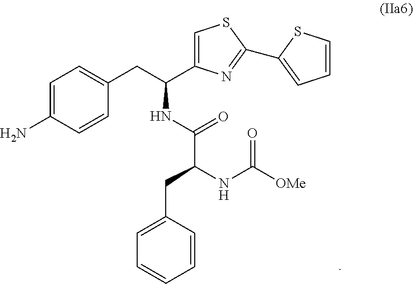 [SMILES: COC(=O)N[C@@H](Cc1ccccc1)C(=O)N[C@@H](Cc1ccc(N)cc1)c1csc(-c2cccs2)n1]

Embodiment C23. The process of any one of embodiments C1-C22, wherein the reaction mixture further comprises an azoxy compound, wherein the azoxy compound is a reduction congener of the nitro compound.

Embodiment C24. The process of embodiment C23, wherein the azoxy compound is:

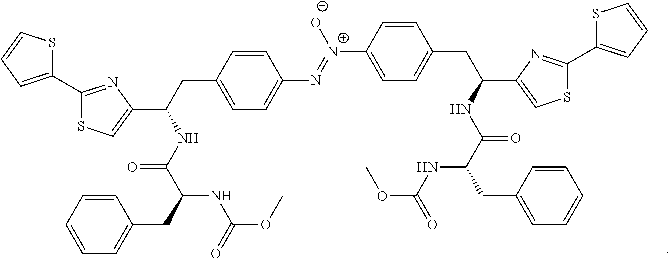 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

Embodiment C25. The process of any one of embodiments C1-C24, wherein the solvent comprises 2-methyltetrahydrofuran.

Embodiment C26. The process of any one of embodiments C1-C25, wherein the reducing comprises contacting the nitro compound with a catalyst in the presence of H₂.

Embodiment C27. The process of any one of embodiments C1-C26, wherein the reducing comprises contacting the nitro compound with a metal-containing catalyst in the presence of H₂.

Embodiment C28. The process of any one of embodiments C1-C27, wherein the reducing comprises contacting the nitro compound with a palladium-based catalyst in the presence of H₂.

Embodiment C29. The process of any one of embodiments C1-C28, wherein the reducing comprises contacting the nitro compound with a palladium on carbon in the presence of H₂.

Embodiment C30. The process of any one of embodiments C1-C29, wherein the reducing comprises contacting the nitro compound with palladium on carbon in the presence of H₂ wherein the palladium on carbon comprises from about 3% to about 8% palladium by weight.

Embodiment C31. The process of any one of embodiments C1-C30, wherein the reducing comprises contacting the nitro compound with palladium on carbon in the presence of H₂, wherein the palladium on carbon comprises about 5% palladium by weight.

Embodiment C32. The process of any one of embodiments C1-C31, wherein the reducing is conducted at a temperature of from about 35° C. to about 55° C.

Embodiment C33. The process of any one of embodiments C1-C32, wherein the reducing is conducted at a temperature of about 45° C.

Embodiment D1. A process comprising contacting an acid of formula (V):

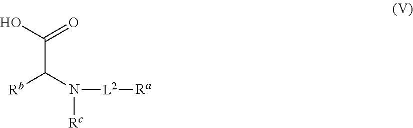

with an amine compound of formula (VI):

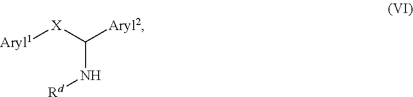

or a salt thereof,
in presence of an amide coupling reagent and a solvent to provide a reaction mixture, the reaction mixture comprising an amide of formula (VII):

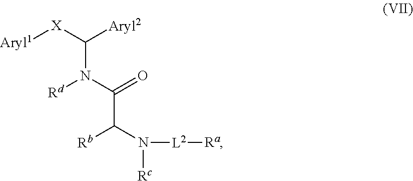

wherein
Aryl¹ is an aryl group which is substituted or unsubstituted;
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond;
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted, wherein a solubility of the solvent in water is less than about 20 grams of the solvent per 100 grams of water at 20° C.

Embodiment D2. The process of embodiment D1, wherein:
Aryl¹ is substituted or unsubstituted phenyl;
Aryl² is substituted or unsubstituted heteroaryl; and
X is alkylene.

Embodiment D3. The process of embodiment D1 or embodiment D2, wherein:
Aryl¹ is substituted phenyl;
Aryl² is substituted heteroaryl; and
X is methylene.

Embodiment D4. The process of any one of embodiments D1-D3, wherein:
i) the acid is of formula (Va):

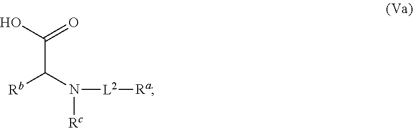

ii) the amine is of formula (VIa)

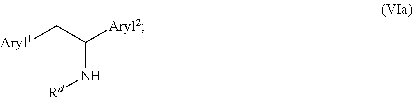

and
iii) the amide is of formula (VIIa)

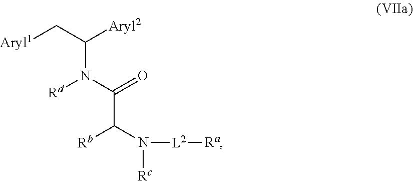

wherein:
Aryl¹ is para-substituted phenyl;
Aryl² is substituted heteroaryl;
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

Embodiment D5. The process of any one of embodiments D1-D4, wherein:
Aryl¹ is para-substituted phenyl;
Aryl² is a substituted thiazole moiety;
X is methylene;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

Embodiment D6. The process of any one of embodiments D1-D5, wherein Aryl² is:

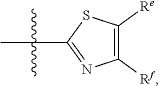

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment D7. The process of embodiment D6, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment D8. The process of embodiment D6, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment D9. The process of any one of embodiments D6-D8, wherein:
Aryl¹ is 4-nitrophenyl;
R^{a} is alkyl, which is substituted or unsubstituted;
R^{b} is arylalkyl, which is substituted or unsubstituted;
R^{e} is H; and
R^{f} is heteroaryl.

Embodiment D10. The process of any one of embodiments D1-D9, wherein:
i) the acid is of formula (Va):

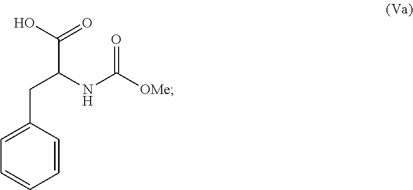 [SMILES: COC(=O)NC(Cc1ccccc1)C(=O)O]

ii) the amine is of formula (VIa):

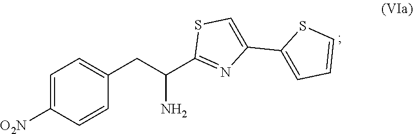 [SMILES: NC(Cc1ccc([N+](=O)[O-])cc1)c1nc(-c2cccs2)cs1]

and
iii) the amide is of formula (VIIa):

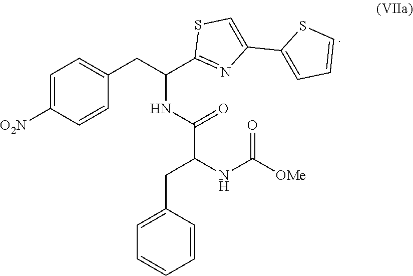 [SMILES: COC(=O)NC(Cc1ccccc1)C(=O)NC(Cc1ccc([N+](=O)[O-])cc1)c1nc(-c2cccs2)cs1]

Embodiment D11. The process of any one of embodiments D1-D10, wherein:
i) the acid is of formula (Va1):

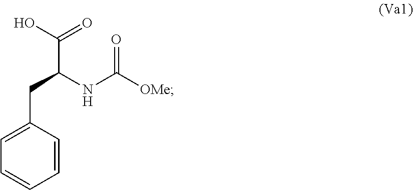 [SMILES: COC(=O)N[C@@H](Cc1ccccc1)C(=O)O]

ii) the amine is of formula (VIa1):

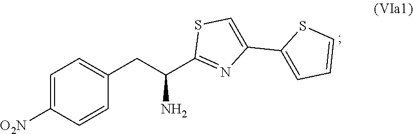 [SMILES: N[C@@H](Cc1ccc([N+](=O)[O-])cc1)c1nc(-c2cccs2)cs1]

and
iii) the amide is of formula (VIIa1):

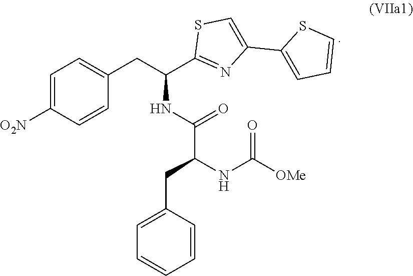 [SMILES: COC(=O)N[C@@H](Cc1ccccc1)C(=O)N[C@@H](Cc1ccc([N+](=O)[O-])cc1)c1nc(-c2cccs2)cs1]

Embodiment D12. The process of any one of embodiments D1-D5, wherein Aryl² is:

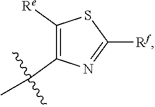

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment D13. The process of embodiment D12, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment D14. The process of embodiment D12, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment D15. The process of any one of embodiments D12-D14, wherein:
Aryl¹ is 4-nitrophenyl;
R^{a} is alkyl, which is substituted or unsubstituted;
R^{b} is arylalkyl, which is substituted or unsubstituted;
R^{e} is H; and
R^{f} is alkyl.

Embodiment D16. The process of any one of embodiments D1-D5 and D12-D14, wherein:
i) the acid is of formula (Va1):

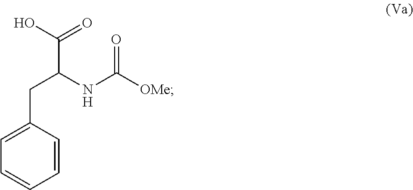 [SMILES: COC(=O)NC(Cc1ccccc1)C(=O)O]

ii) the amine is of formula (VIb):

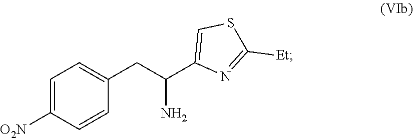 [SMILES: CCc1nc(C(N)Cc2ccc([N+](=O)[O-])cc2)cs1]

and
iii) the amide is of formula (VIIb):

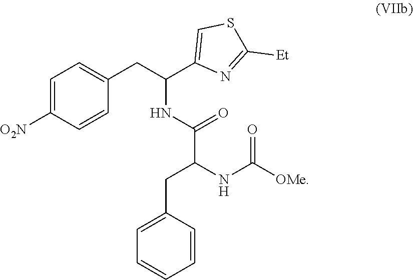 [SMILES: CCc1nc(C(Cc2ccc([N+](=O)[O-])cc2)NC(=O)C(Cc2ccccc2)NC(=O)OC)cs1]

Embodiment D17. The process of any one of embodiments D1-D5 and D12-D15, wherein:
i) the acid is of formula (Va1):

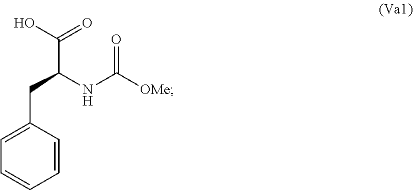 [SMILES: COC(=O)N[C@@H](Cc1ccccc1)C(=O)O]

ii) the amine is of formula (VIb1):

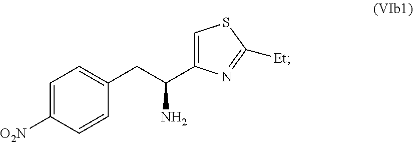 [SMILES: CCc1nc([C@@H](N)Cc2ccc([N+](=O)[O-])cc2)cs1]

and
iii) the amide is of formula (VIIb1):

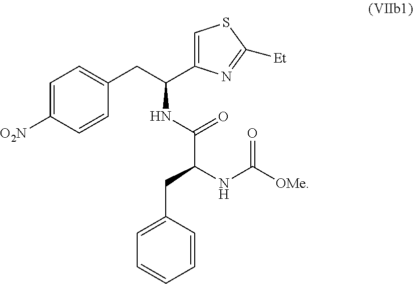 [SMILES: CCc1nc([C@H](Cc2ccc([N+](=O)[O-])cc2)NC(=O)[C@H](Cc2ccccc2)NC(=O)OC)cs1]

Embodiment D18. The process of any one of embodiments D12-D14, wherein:
Aryl¹ is 4-nitrophenyl;
R^{a} is alkyl, which is substituted or unsubstituted;
R^{b} is arylalkyl, which is substituted or unsubstituted;
R^{e} is H; and
R^{f} is heteroaryl.

Embodiment D19. The process of any one of embodiments D1-D5, D12-D14, and D18, wherein:
i) the acid is of formula (Va):

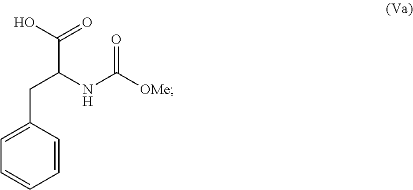 [SMILES: COC(=O)NC(Cc1ccccc1)C(=O)O]

ii) the amine is of formula (VIc):

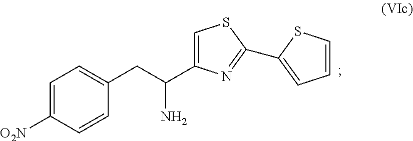 [SMILES: NC(Cc1ccc([N+](=O)[O-])cc1)c1csc(-c2cccs2)n1]

and
iii) the amide is of formula (VIIc):

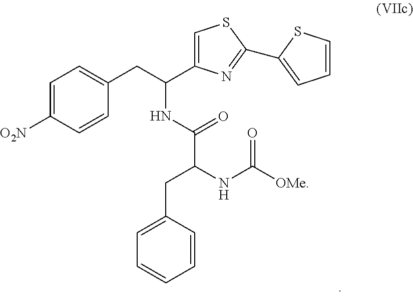 [SMILES: COC(=O)NC(Cc1ccccc1)C(=O)NC(Cc1ccc([N+](=O)[O-])cc1)c1csc(-c2cccs2)n1]

Embodiment D20. The process of any one of embodiments D1-D5, D12-D14, D18, and D19, wherein:
i) the acid is of formula (Va1):

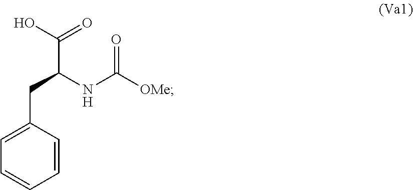 [SMILES: COC(=O)N[C@@H](Cc1ccccc1)C(=O)O]

ii) the amine is of formula (VIc1):

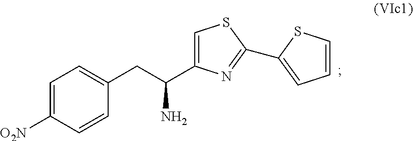 [SMILES: N[C@@H](Cc1ccc([N+](=O)[O-])cc1)c1csc(-c2cccs2)n1]

and
iii) the amide is of formula (VIIc1):

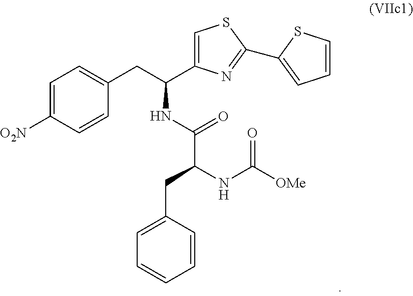 [SMILES: COC(=O)N[C@@H](Cc1ccccc1)C(=O)N[C@@H](Cc1ccc([N+](=O)[O-])cc1)c1csc(-c2cccs2)n1]

Embodiment D21. The process of any one of embodiments D1-D20, wherein the amine has a solubility of less than about 50 mg/mL in the solvent at a temperature of from about 55° C. to about 60° C.

Embodiment D22. The process of any one of embodiments D1-D20, wherein the amide has a solubility of less than about 30 mg/mL in the solvent at a temperature of from about 60° C. to about 65° C.

Embodiment D23. The process of any one of embodiments D1-D22, wherein the solvent comprises 2-methyltetrahydrofuran.

Embodiment D24. The process of any one of embodiments D1-D23, wherein the amide coupling reagent is a substituted 1,3,5-triazine.

Embodiment D25. The process of any one of embodiments D1-D24, wherein the amide coupling reagent is 4-(4,6-dimethoxy-1,3,5-triazin-2-yl)-4-methyl-morpholinium chloride.

Embodiment D26. The process of any one of embodiments D1-D24, wherein the amide coupling reagent is 4-(4,6-dimethoxy-1,3,5-triazin-2-yl)-4-methyl-morpholinium tetrafluoroborate.

Embodiment D27. The process of any one of embodiments D1-D24, wherein the amide coupling reagent is 2-chloro-4,6-dimethoxy-1,3,5-triazine, wherein the contacting is conducted in the presence of a base.

Embodiment D28. The process of embodiment D27, wherein the base comprises an amine moiety.

Embodiment D29. The process of embodiment D27 or embodiment D28, wherein the base is N-methylmorpholine.

Embodiment E1. A process comprising contacting a quantity of L-phenylalanine with a quantity of methyl chloroformate in presence of a base and a solvent to form a reaction mixture, wherein the reaction mixture comprises a quantity of a compound of formula (Va1):

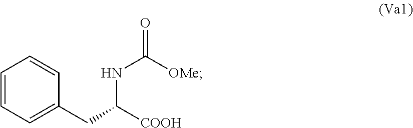 [SMILES: COC(=O)N[C@@H](Cc1ccccc1)C(=O)O]

and a quantity of a side-product of formula (VIII):

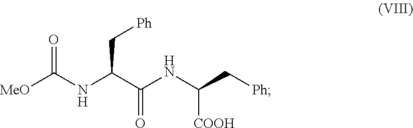 [SMILES: COC(=O)N[C@@H](Cc1ccccc1)C(=O)N[C@@H](Cc1ccccc1)C(=O)O]

and
wherein an area/area ratio of the quantity of the compound of formula (Va1) to the quantity of the side-product of formula (VIII) is at least about 95:5 as determined by a liquid chromatography assay, wherein the assay is performed on a sample of the reaction mixture that is obtained at least about 1 hour after initiation of the contacting, and wherein the quantity of methyl chloroformate is at least 1 kg.

Embodiment E2. The process of embodiment E1, wherein the area/area ratio of the quantity of the compound of formula (Va1) to the quantity of the side-product of formula (VIII) in the reaction mixture at least about 99:1.

Embodiment E3. The process of embodiment E1, wherein the area/area ratio of the quantity of the compound of formula (Va1) to the quantity of the side-product of formula (VIII) in the reaction mixture at least about 99.5:0.5.

Embodiment E4. The process of embodiment E1, wherein the area/area ratio of the quantity of the compound of formula (Va1) to the quantity of the side-product of formula (VIII) in the reaction mixture at least about 99.8:0.2.

Embodiment E5. The process of embodiment E1, wherein the area/area ratio of the quantity of the compound of formula (Va1) to the quantity of the side-product of formula (VIII) in the reaction mixture at least about 99.9:0.1.

Embodiment E6. The process of any one of embodiments E1-E5, wherein the quantity of methyl chloroformate is less than about 1.5 molar equivalents with respect to the quantity of L-phenylalanine.

Embodiment E7. The process of any one of embodiments E1-E5, wherein the quantity of methyl chloroformate is from about 1.3 to about 1.4 molar equivalents with respect to the quantity of L-phenylalanine.

Embodiment E8. The process of any one of embodiments E1-E7, wherein a quantity of the base is at least about 2 molar equivalents with respect to the quantity of L-phenylalanine.

Embodiment E9. The process of any one of embodiments E1-E7, wherein a quantity of the base is from about 2.8 to about 3.3 molar equivalents with respect to the quantity of L-phenylalanine.

Embodiment E10. The process of any one of embodiments E1-E9, wherein the contacting comprises:
(i) dissolving the quantity of L-phenylalanine and the base in the solvent to provide a basic solution; and
(ii) adding the quantity of the methyl chloroformate to the basic solution to form the reaction mixture.

Embodiment E11. The process of embodiment E10, wherein the reaction mixture is maintained at a temperature of less than about 10° C. during (ii).

Embodiment E12. The process of embodiment E10, wherein the reaction mixture is maintained at a temperature from about −20° C. to about 0° C. during (ii).

Embodiment E13. The process of any one of embodiments E10-E12, wherein the adding of (ii) occurs at rate of less than about 2 molar equivalents of methyl chloroformate with respect to the quantity of L-phenylalanine per hour.

Embodiment E14. The process of any one of embodiments E10-E12, wherein the adding of (ii) occurs at rate from about 0.05 to about 1 molar equivalents of methyl chloroformate with respect to the quantity of L-phenylalanine per hour.

Embodiment E15. The process of any one of embodiments E1-E14, further comprising adding an organic solvent that is immiscible with water to the reaction mixture when less than about 95% of the quantity of L-phenylalanine has been consumed.

Embodiment E16. The process of embodiment E15, wherein the organic solvent that is immiscible with water is methyl tert-butyl ether (MTBE).

Embodiment F1. A composition comprising:
a) a compound of formula (Ia6):

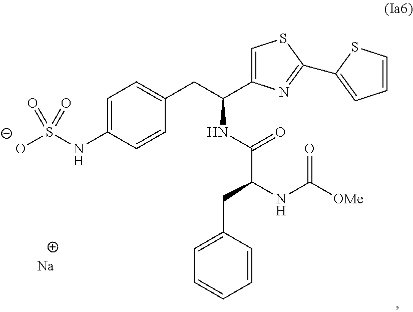 [SMILES: COC(=O)N[C@@H](Cc1ccccc1)C(=O)N[C@@H](Cc1ccc(NS(=O)(=O)[O-])cc1)c1csc(-c2cccs2)n1 ; [Na+]]

and
b) a compound of formula (IIa6):

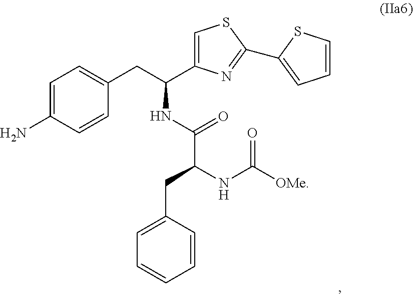 [SMILES: COC(=O)N[C@@H](Cc1ccccc1)C(=O)N[C@@H](Cc1ccc(N)cc1)c1csc(-c2cccs2)n1]

in a mixture, wherein the compound of formula (Ia6) forms at least about 99.0% (a/a) of the composition as determined by UPLC, and wherein the compound of formula (IIa6) forms from about 0.001% to about 0.5% (a/a) of the composition as determined by UPLC, wherein the composition is substantially free of solvent.

Embodiment F2. The composition of embodiment F1, wherein the compound of formula (Ia6) forms at least about 99.2% (a/a) of the composition as determined by UPLC.

Embodiment F3. The composition of embodiment F1, wherein the compound of formula (Ia6) forms at least about 99.3% (a/a) of the composition as determined by UPLC.

Embodiment F4. The composition of embodiment F1, wherein the compound of formula (Ia6) forms at least about 99.4% (a/a) of the composition as determined by UPLC.

Embodiment F5. The composition of embodiment F1, wherein the compound of formula (Ia6) forms from about 99.3% to about 99.5% (a/a) of the composition as determined by UPLC.

Embodiment F6. The composition of any one of embodiments F1-F5, wherein the compound of formula (IIa6) forms from about 0.001% to about 0.1% (a/a) of the composition as determined by UPLC.

Embodiment F7. The composition of any one of embodiments F1-F5, wherein the compound of formula (IIa6) forms from about 0.01% to about 0.1% (a/a) of the composition as determined by UPLC.

Embodiment F8. The composition of any one of embodiments F1-F5, wherein the compound of formula (IIa6) forms from about 0.01% to about 0.03% (a/a) of the composition as determined by UPLC.

Embodiment F9. The composition of any one of embodiments F1-F8, comprising no more than 100 ppm of a compound of formula (G-2):

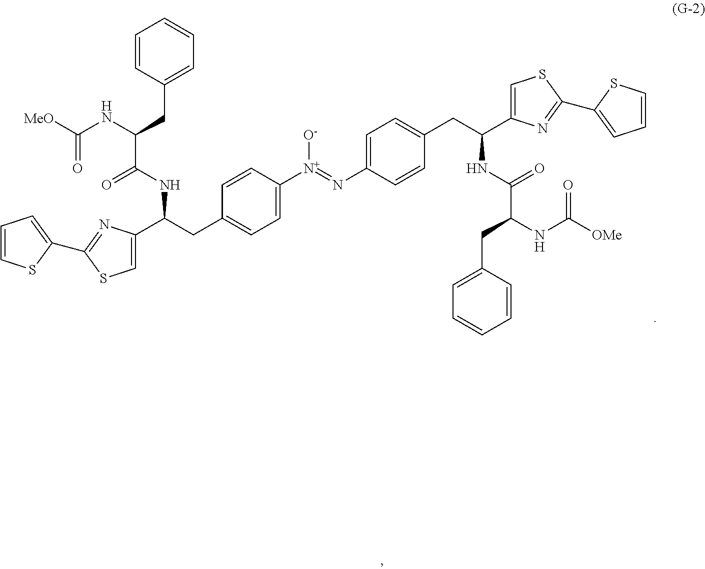 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

Embodiment F10. The composition of embodiment F9, comprising no more than 1 ppm of the compound of formula (G-2).

Embodiment G1. A compound of formula (G-2):

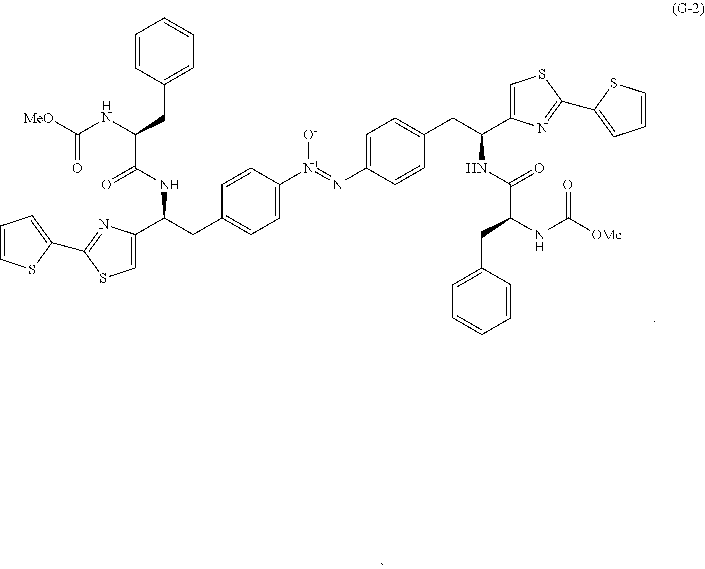 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

Embodiment H1. A process comprising:
a) contacting a compound of formula (IIa6) with a sulfur trioxide source to form a compound of formula (III):

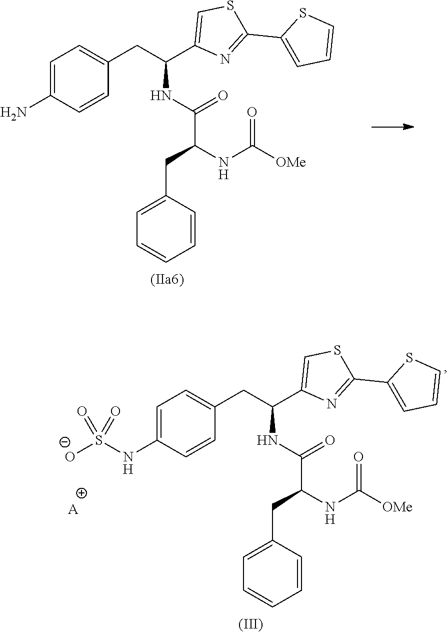

wherein A+ is an organic cation;
b) contacting the compound of formula (III) with a sodium cation source to form a reaction mixture comprising a compound of formula (Ia6):

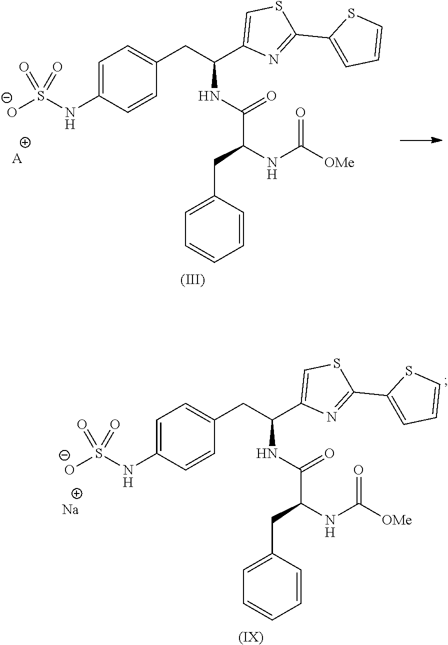

c) treating the reaction mixture to form a composition comprising the compound of formula (Ia6),
wherein the compound of formula (IX) forms at least about 99.0% (a/a) of the composition as determined by UPLC, and wherein the compound of formula (IIa6) forms from about 0.001% to about 0.5% (a/a) of the composition as determined by UPLC, wherein the composition is substantially free of solvent.

Embodiment H2. The process of embodiment H1, wherein the compound of formula (IX) forms at least about 99.2% (a/a) of the composition as determined by UPLC.

Embodiment H3. The process of embodiment H1, wherein the compound of formula (IX) forms at least about 99.3% (a/a) of the composition as determined by UPLC.

Embodiment H4. The process of embodiment H1, wherein the compound of formula (IX) forms at least about 99.4% (a/a) of the composition as determined by UPLC.

Embodiment H5. The process of embodiment H1, wherein the compound of formula (IX) forms from about 99.3% to about 99.5% (a/a) of the composition as determined by UPLC.

Embodiment H6. The process of any one of embodiments H1-H5, wherein the compound of formula (IIa6) forms from about 0.001% to about 0.1% (a/a) of the composition as determined by UPLC.

Embodiment H7. The process of any one of embodiments H1-H5, wherein the compound of formula (IIa6) forms from about 0.01% to about 0.1% (a/a) of the composition as determined by UPLC.

Embodiment H8. The process of any one of embodiments H1-H5, wherein the compound of formula (IIa6) forms from about 0.01% to about 0.03% (a/a) of the composition as determined by UPLC.

Embodiment H9. The process of any one of embodiments H1-H8, wherein the contacting of b) comprises contacting the compound of formula (III) with a basic solution, wherein the basic solution comprises a C1-C6 sodium alkoxide base at an amount of no more than about 15% (w/w), to provide the reaction mixture.

Embodiment H10. The process of any one of embodiments H1-H9, wherein the contacting of b) comprises contacting the compound of formula (III) with a basic solution, wherein the basic solution comprises sodium methoxide at an amount of no more than about 15% (w/w), to provide the reaction mixture.

Embodiment H11. The process of any one of embodiments H1-H9, wherein the contacting of b) comprises contacting the compound of formula (III) with a basic solution, wherein the basic solution comprises sodium methoxide at an amount of about 0.1% to about 5% (w/w), to provide the reaction mixture.

Embodiment H12. The process of any one of embodiments H1-H8, wherein the contacting of b) comprises contacting the compound of formula (III) with a basic solution, wherein the basic solution comprises no more than about 0.5% (w/w) of a C1-C6 sodium alkoxide base, to provide an intermediate mixture, and then contacting the intermediate mixture with a second basic solution, wherein the second basic solution comprises a C1-C6 sodium alkoxide base at an amount of no more than about 15% (w/w) to provide the reaction mixture.

Embodiment H13. The process of any one of embodiments H1-H8, wherein the contacting of b) comprises contacting the compound of formula (III) with a basic solution provide an intermediate mixture, and then contacting the intermediate mixture with a second basic solution, wherein the basic solution comprises sodium methoxide at an amount of no more than about 15% (w/w), to provide the reaction mixture.

Embodiment H14. The process of any one of embodiments H1-H8, wherein the contacting of b) comprises contacting the compound of formula (III) with a basic solution provide an intermediate mixture, and then contacting the intermediate mixture with a second basic solution, wherein the second basic solution comprises sodium methoxide at an amount of about 0.1% to about 5% (w/w) to provide the reaction mixture.

Embodiment H15. The process of any one of embodiments H1-H8, wherein the contacting of b) comprises contacting the compound of formula (III) with a basic solution, wherein the basic solution comprises sodium methoxide at an amount of about 0.01% to about 0.5% (w/w), to provide an intermediate mixture, and then contacting the intermediate mixture with a second basic solution, wherein the second basic solution comprises sodium methoxide at an amount of about 0.1% to about 5% (w/w), to provide the reaction mixture.

Embodiment H16. The process of any one of embodiments H1-H15, wherein the compound of formula (IIa6) is contacted with a sulfur trioxide source in the presence of a solvent and a base, wherein the solvent has a water content below 0.1% w/w.

Embodiment H17. The process of any one of embodiments H1-H16, wherein the treating of c) comprises filtering the reaction mixture to provide a retentate, washing the retentate with a solvent, and drying the retentate to provide the composition.

Embodiment H18. The process of any one of embodiments H1-H17, wherein a chemical yield of the compound of formula (IX) is at least about 60%, with respect to an initial quantity of the compound of formula (III).

Embodiment H19. The process of any one of embodiments H1-H17, wherein a chemical yield of the compound of formula (IX) is at least about 70%, with respect to an initial quantity of the compound of formula (III).

Embodiment H20. The process of any one of embodiments H1-H17, wherein a chemical yield of the compound of formula (IX) is from about 70% to about 90%, with respect to an initial quantity of the compound of formula (III).

Embodiment H21. The process of any one of embodiments H1-H20, wherein a chemical yield of the compound of formula (IX) is at least about 10 kg.

Embodiment H22. The process of any one of embodiments H1-H20, wherein a chemical yield of the compound of formula (IX) is from about 10 kg to about 100 kg.

Embodiment H23. The process of any one of embodiments H1-H20, wherein a chemical yield of the compound of formula (IX) is from about 10 kg to about 20 kg.

Embodiment H24. The process of any one of embodiments H1-H23, wherein the sulfur trioxide source is a complex of sulfur trioxide and an organic molecule that comprises a nitrogen atom.

Embodiment H25. The process of any one of embodiments H1-H24, wherein the sulfur trioxide source is a complex of sulfur trioxide and trimethylamine.

Embodiment H26. The process of any one of embodiments H1-H17, wherein a chemical yield of the compound of formula (III) is at least about 80%, with respect to the compound of formula (IIa6).

Embodiment H27. The process of any one of embodiments H1-H17, wherein a chemical yield of the compound of formula (III) is at least about 90%, with respect to the compound of formula (IIa6).

Embodiment H28. The process of any one of embodiments H1-H17, wherein a chemical yield of the compound of formula (III) is at least about 95%, with respect to the compound of formula (IIa6).

Embodiment H29. The process of any one of embodiments H1-H17, wherein a chemical yield of the compound of formula (III) is from about 95% to about 99%, with respect to the compound of formula (IIa6).

Embodiment H30. The process of any one of embodiments H1-H8, wherein the sodium cation source comprises an alkoxide salt.

Embodiment H31. The process of any one of embodiments H1-H8, wherein the sodium cation source comprises sodium methoxide.

Embodiment I1. A process comprising reducing a compound of formula (IVa6):

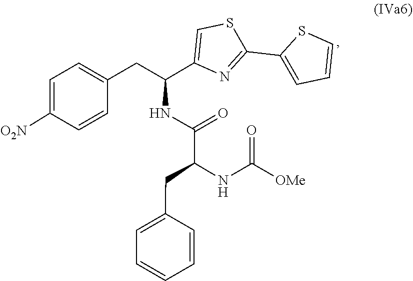 [SMILES: COC(=O)N[C@@H](Cc1ccccc1)C(=O)N[C@@H](Cc1ccc([N+](=O)[O-])cc1)c1csc(-c2cccs2)n1]

in presence of a solvent to provide a reaction mixture, wherein the reaction mixture comprises a compound of formula (IIa6):

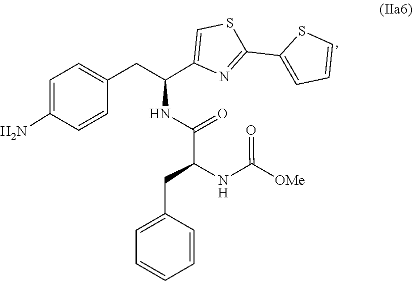 [SMILES: COC(=O)N[C@@H](Cc1ccccc1)C(=O)N[C@@H](Cc1ccc(N)cc1)c1csc(-c2cccs2)n1]

wherein a solubility of the solvent in water is less than about 20 grams of the solvent per 100 grams of water at 20° C.

Embodiment I2. The process of embodiment I1, wherein the solvent comprises 2-methyltetrahydrofuran.

Embodiment I3. The process of embodiment I1 or embodiment 12, wherein the reducing comprises contacting the nitro compound with a catalyst in the presence of H₂.

Embodiment I4. The process of any one of embodiments I1-I3, wherein the reducing comprises contacting the nitro compound with a metal-containing catalyst in the presence of H₂.

Embodiment I5. The process of any one of embodiments I1-I4, wherein the reducing comprises contacting the nitro compound with a palladium-based catalyst in the presence of H₂.

Embodiment I6. The process of any one of embodiments I1-I5, wherein the reducing comprises contacting the nitro compound with a palladium on carbon in the presence of H₂.

Embodiment I7. The process of any one of embodiments I1-I6, wherein the reducing comprises contacting the nitro compound with palladium on carbon in the presence of H₂ wherein the palladium on carbon comprises from about 3% to about 8% palladium by weight.

Embodiment I8. The process of any one of embodiments I1-I7, wherein the reducing comprises contacting the nitro compound with palladium on carbon in the presence of H₂, wherein the palladium on carbon comprises about 5% palladium by weight.

Embodiment I9. The process of any one of embodiments I1-I8, wherein the reducing is conducted at a temperature of from about 35° C. to about 55° C.

Embodiment I10. The process of any one of embodiments I1-I8, wherein the reducing is conducted at a temperature of about 45° C.

Embodiment J1. A process comprising contacting a compound of formula (V):

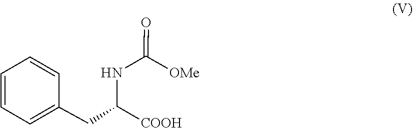 [SMILES: COC(=O)N[C@@H](Cc1ccccc1)C(=O)O]

with a compound of formula (VI):

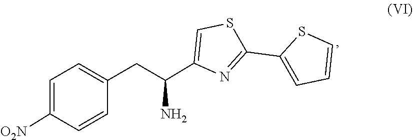 [SMILES: N[C@@H](Cc1ccc([N+](=O)[O-])cc1)c1csc(-c2cccs2)n1]

or a salt thereof,
in presence of an amide coupling reagent and a solvent to provide a reaction mixture, the reaction mixture comprising a compound of formula (IV):

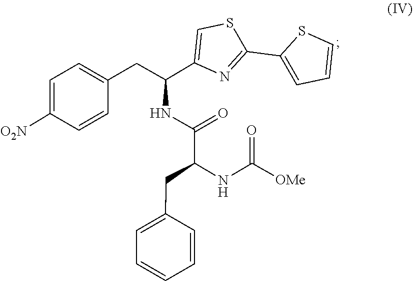 [SMILES: COC(=O)N[C@@H](Cc1ccccc1)C(=O)N[C@@H](Cc1ccc([N+](=O)[O-])cc1)c1csc(-c2cccs2)n1]

wherein a solubility of the solvent in water is less than about 20 grams of the solvent per 100 grams of water at 20° C.

Embodiment J2. The process of embodiment J1, wherein the compound of formula (VI) has a solubility of less than about 50 mg/mL in the solvent at a temperature of from about 55° C. to about 60° C.

Embodiment J3. The process of embodiment J1, wherein the compound of formula (IV) has a solubility of less than about 30 mg/mL in the solvent at a temperature of from about 60° C. to about 65° C.

Embodiment J4. The process of any one of embodiments J1-J3, wherein the solvent comprises 2-methyltetrahydrofuran.

Embodiment J5. The process of any one of embodiments J1-J4, wherein the amide coupling reagent is a substituted 1,3,5-triazine.

Embodiment J6. The process of any one of embodiments J1-J5, wherein the amide coupling reagent is 4-(4,6-dimethoxy-1,3,5-triazin-2-yl)-4-methyl-morpholinium chloride.

Embodiment J7. The process of any one of embodiments J1-J5, wherein the amide coupling reagent is 4-(4,6-dimethoxy-1,3,5-triazin-2-yl)-4-methyl-morpholinium tetrafluoroborate.

Embodiment J8. The process of any one of embodiments J1-J5, wherein the amide coupling reagent is 2-chloro-4,6-dimethoxy-1,3,5-triazine, wherein the contacting is conducted in the presence of a base.

Embodiment J9. The process of embodiment J8, wherein the base comprises an amine moiety.

Embodiment J10. The process of embodiment J8 or embodiment J9, wherein the base is N-methylmorpholine.

1. A pharmaceutical composition comprising a mixture of a Tie-2 modulator and a second compound, wherein:
(a) each of the Tie-2 modulator and the second compound has a core structure and a nitrogen atom substituent bound to the core structure at a position on the core structure;
(b) the core structure of the Tie-2 modulator is identical to the core structure of the second compound;
(c) the position on the core structure of the Tie-2 modulator to which the nitrogen atom substituent is bound is identical to the position on the core structure of the second compound to which the nitrogen atom substituent is bound;
(d) the nitrogen atom substituent of the Tie-2 modulator is —N(H)(E), wherein E is a group that contains a sulfur atom bound to the nitrogen atom;
(e) the nitrogen atom substituent of the second compound is —NH₂; and
(f) the pharmaceutical composition is substantially free of solvent.

2. The pharmaceutical composition of claim 1, wherein the Tie-2 modulator forms at least about 99.0% (a/a) of the mixture as determined by a liquid chromatography assay, and wherein the second compound forms from about 0.001% to about 0.5% (a/a) of the mixture as determined by the liquid chromatography assay.

3. The pharmaceutical composition of claim 1, wherein the nitrogen atom substituent of the Tie-2 modulator is a sulfamate group.

4. The pharmaceutical composition of claim 1, wherein the second compound is a desulfonylation congener of the Tie-2 modulator.

5. The pharmaceutical composition of claim 1, wherein:
the Tie-2 modulator has a structure of Q-Z; and
the second compound has a structure of W—Z, wherein
Q is 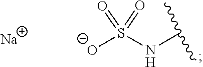 [SMILES: [Na+] ; *NS(=O)(=O)[O-]]
W is H₂N—; and
each Z is the core structure.

6. The pharmaceutical composition of claim 5, wherein each Z is: 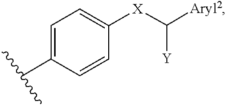
wherein
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond; and
Y is H, aryl, heteroaryl, NH(aryl), NH(heteroaryl), or NHCOR^{g}, any of which is substituted or unsubstituted, or 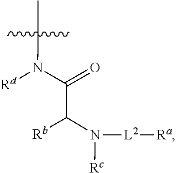
wherein:
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted.

7. The pharmaceutical composition of claim 6, wherein:
Aryl² is substituted or unsubstituted heteroaryl; and
X is alkylene.

8. (canceled)

9. The pharmaceutical composition of claim 5, wherein each Z is: 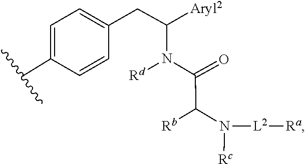
wherein:
Aryl² is substituted heteroaryl;
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

10. The pharmaceutical composition of claim 9, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

11. The pharmaceutical composition of claim 10, wherein Aryl² is: 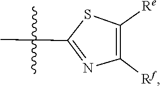
wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

12-14. (canceled)

15. The pharmaceutical composition of claim 6, wherein each Z is:

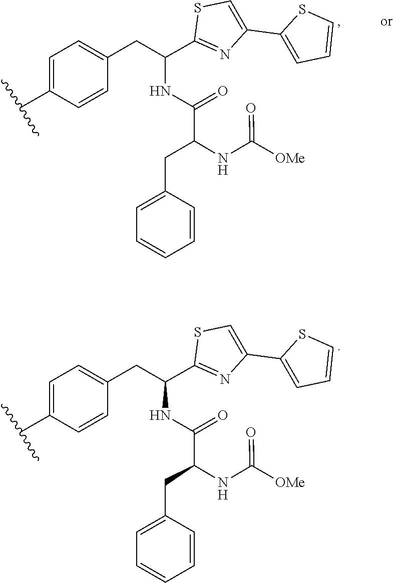 [SMILES: *c1ccc(CC(NC(=O)C(Cc2ccccc2)NC(=O)OC)c2nc(-c3cccs3)cs2)cc1 ; *c1ccc(C[C@H](NC(=O)[C@H](Cc2ccccc2)NC(=O)OC)c2nc(-c3cccs3)cs2)cc1]

16. (canceled)

17. The pharmaceutical composition of claim 10, wherein Aryl² is: 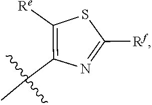
wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a ureido group, a thioether group, a thioester group, a thioacid group, aryl, arylalkyl, heterocyclyl, heterocyclylalkyl, heteroaryl, or heteroarylalkyl, any of which is substituted or unsubstituted.

18. The pharmaceutical composition of claim 17, wherein:
R^{c} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

19-20. (canceled)

21. The pharmaceutical composition of claim 6, wherein each Z is:

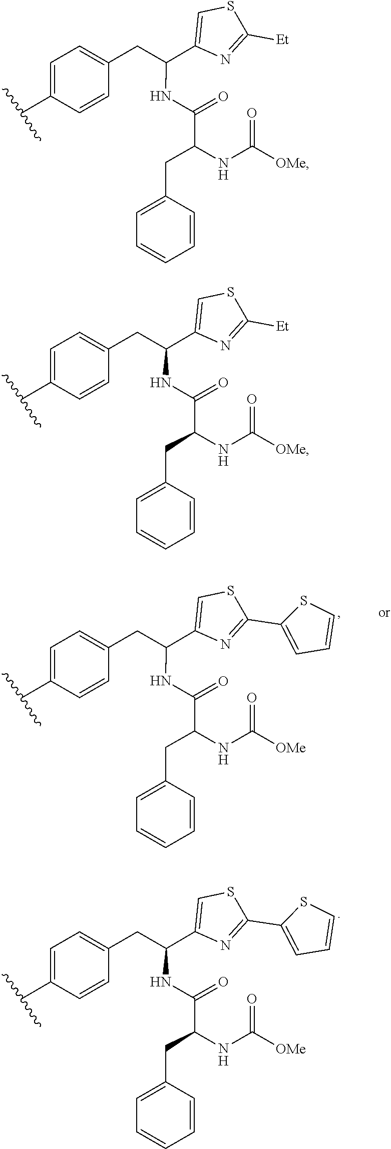 [SMILES: *c1ccc(CC(NC(=O)C(Cc2ccccc2)NC(=O)OC)c2csc(CC)n2)cc1 ; *c1ccc(C[C@H](NC(=O)[C@H](Cc2ccccc2)NC(=O)OC)c2csc(CC)n2)cc1 ; *c1ccc(CC(NC(=O)C(Cc2ccccc2)NC(=O)OC)c2csc(-c3cccs3)n2)cc1 ; *c1ccc(C[C@H](NC(=O)[C@H](Cc2ccccc2)NC(=O)OC)c2csc(-c3cccs3)n2)cc1]

22-25. (canceled)

26. The pharmaceutical composition of claim 2, wherein the Tie-2 modulator forms at least about 99.2% to about 99.5% (a/a) of the mixture as determined by the liquid chromatography assay.

27-29. (canceled)

30. The pharmaceutical composition of claim 2, wherein the second compound forms from about 0.001% to about 0.1% (a/a) of the mixture as determined by the liquid chromatography assay.

31-34. (canceled)

35. The pharmaceutical composition of claim 1, wherein the composition comprises no more than about 100 ppm of a third compound as determined by HPLC, wherein the third compound comprises an azoxy moiety.

36-37. (canceled)

38. The pharmaceutical composition of claim 5, wherein the composition comprises no more than about 100 ppm of a third compound as determined by HPLC, wherein the third compound has a structure of Z-J-Z, wherein J is

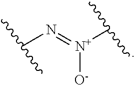 [SMILES: */N=[N+](/*)[O-]]

39. (canceled)

40. A process for preparing a composition, the process comprising:
(i) contacting an initial quantity of an amine with a sulfur trioxide source in a solvent to afford a first mixture, wherein the first mixture comprises a quantity of a first ion pair that is a sulfamate anion and an organic cation; and
(ii) contacting the first ion pair with a sodium cation source to provide a second mixture, wherein the second mixture comprises a second ion pair and the amine, wherein the second ion pair is a sodium cation and the sulfamate anion,
wherein the initial quantity of the amine is at least 1 kg, and a ratio of the sulfamate anion to the amine in the second mixture is at least 99:1 (a/a) as determined by a liquid chromatography assay.

41-76. (canceled)

77. The process of claim 40, wherein:
i) the sulfamate anion is of formula (Ia5): 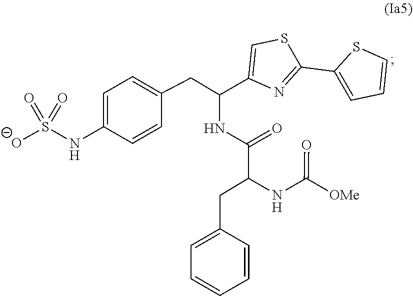 [SMILES: COC(=O)NC(Cc1ccccc1)C(=O)NC(Cc1ccc(NS(=O)(=O)[O-])cc1)c1csc(-c2cccs2)n1]
and
ii) the amine is of formula (IIa5):

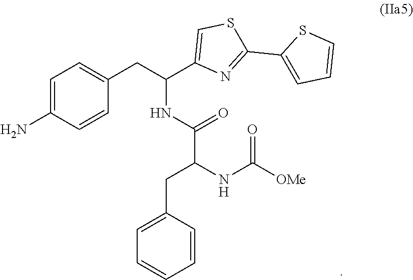 [SMILES: COC(=O)NC(Cc1ccccc1)C(=O)NC(Cc1ccc(N)cc1)c1csc(-c2cccs2)n1]

78-112. (canceled)

113. A process comprising reducing a nitro compound in presence of a solvent to provide a reaction mixture comprising an amino compound, wherein the amino compound is a desulfonylation congener of a Tie-2 modulator, and a solubility of the solvent in water is less than about 20 grams of the solvent per 100 grams of water at 20° C.

114-123. (canceled)

124. The process of claim 113, wherein:
i) the nitro compound is of formula (IVa1): 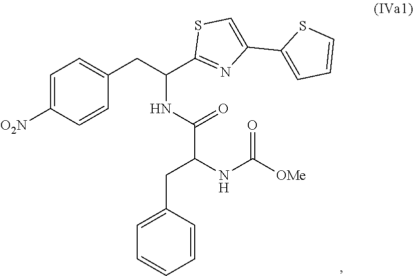 [SMILES: COC(=O)NC(Cc1ccccc1)C(=O)NC(Cc1ccc([N+](=O)[O-])cc1)c1nc(-c2cccs2)cs1]
and
ii) the amine is of formula (IIa1):

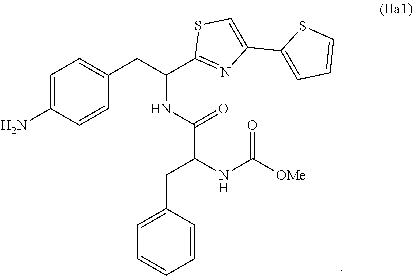 [SMILES: COC(=O)NC(Cc1ccccc1)C(=O)NC(Cc1ccc(N)cc1)c1nc(-c2cccs2)cs1]

125-145. (canceled)

146. A process comprising contacting an acid of formula (V): 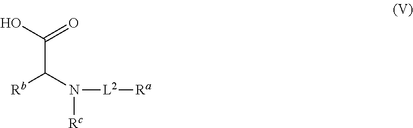
with an amine compound of formula (VI): 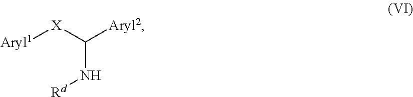
or a salt thereof,
in presence of an amide coupling reagent and a solvent to provide a reaction mixture, the reaction mixture comprising an amide of formula (VII): 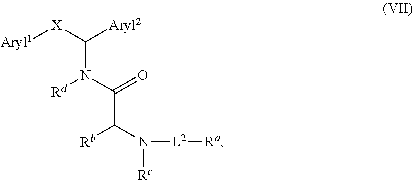
wherein:
Aryl¹ is an aryl group which is substituted or unsubstituted;
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a ureido linkage, a sulfone linkage, any of which is substituted or unsubstituted, or a chemical bond;
L² is alkylene, alkenylene, or alkynylene, any of which is substituted or unsubstituted, or together with the nitrogen atom to which L² is bound forms an amide linkage, a carbamate linkage, a ureido linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}R^{b}, and R^{d} forms a ring that is substituted or unsubstituted;
R^{d} is H or alkyl which is substituted or unsubstituted, or together with any of L², R^{a}R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted, wherein a solubility of the solvent in water is less than about 20 grams of the solvent per 100 grams of water at 20° C.

147-164. (canceled)

165. The process of claim 146, wherein:
i) the acid is of formula (Va1): 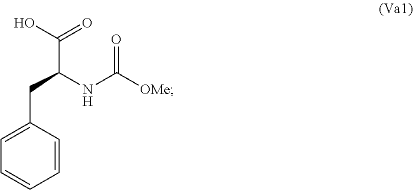 [SMILES: COC(=O)N[C@@H](Cc1ccccc1)C(=O)O]
ii) the amine is of formula (VIc1): 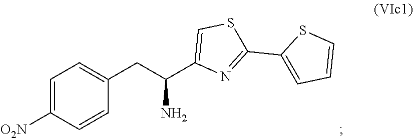 [SMILES: N[C@@H](Cc1ccc([N+](=O)[O-])cc1)c1csc(-c2cccs2)n1]
and
iii) the amide is of formula (VIIc1):

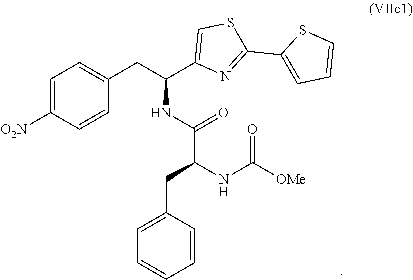 [SMILES: COC(=O)N[C@@H](Cc1ccccc1)C(=O)N[C@@H](Cc1ccc([N+](=O)[O-])cc1)c1csc(-c2cccs2)n1]

166-174. (canceled)

175. A process comprising contacting a quantity of L-phenylalanine with a quantity of methyl chloroformate in presence of a base and a solvent to form a reaction mixture, wherein the reaction mixture comprises a quantity of a compound of formula (Va1): 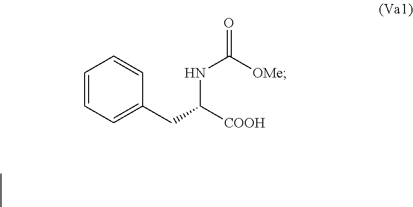
and a quantity of a side-product of formula (VIII): 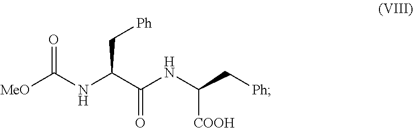 [SMILES: COC(=O)N[C@@H](Cc1ccccc1)C(=O)N[C@@H](Cc1ccccc1)C(=O)O]
and
wherein an area/area ratio of the quantity of the compound of formula (Va1) to the quantity of the side-product of formula (VIII) is at least about 95:5 as determined by a liquid chromatography assay, wherein the assay is performed on a sample of the reaction mixture that is obtained at least about 1 hour after initiation of the contacting, and wherein the quantity of methyl chloroformate is at least 1 kg.

176-183. (canceled)

184. The process of claim 175, wherein the contacting comprises:
(i) dissolving the quantity of L-phenylalanine and the base in the solvent to provide a basic solution; and
(ii) adding the quantity of the methyl chloroformate to the basic solution to form the reaction mixture.

185-190. (canceled)

191. A composition comprising:
a) a compound of formula (Ia6): 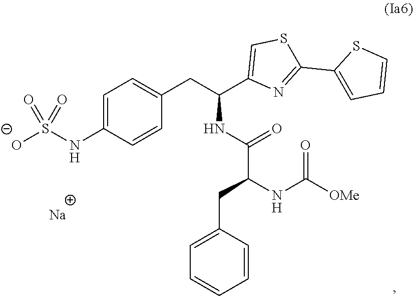 [SMILES: COC(=O)N[C@@H](Cc1ccccc1)C(=O)N[C@@H](Cc1ccc(NS(=O)(=O)[O-])cc1)c1csc(-c2cccs2)n1 ; [Na+]]
and
b) a compound of formula (IIa6): 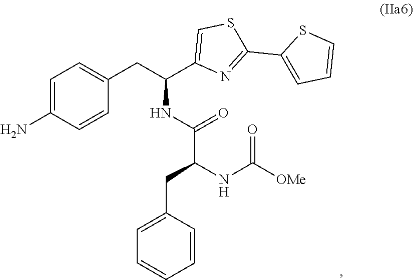 [SMILES: COC(=O)N[C@@H](Cc1ccccc1)C(=O)N[C@@H](Cc1ccc(N)cc1)c1csc(-c2cccs2)n1]
in a mixture, wherein the compound of formula (Ia6) forms at least about 99.0% (a/a) of the composition as determined by UPLC, and wherein the compound of formula (IIa6) forms from about 0.001% to about 0.5% (a/a) of the composition as determined by UPLC, wherein the composition is substantially free of solvent.

192-198. (canceled)

199. The composition of claim 191, comprising no more than 100 ppm of a compound of formula (G-2):

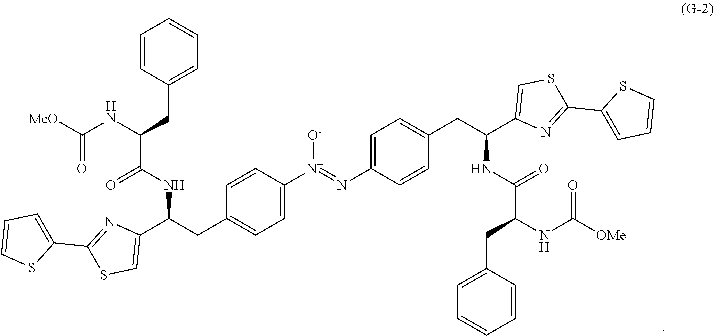 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]

200. A compound of formula (G-2): 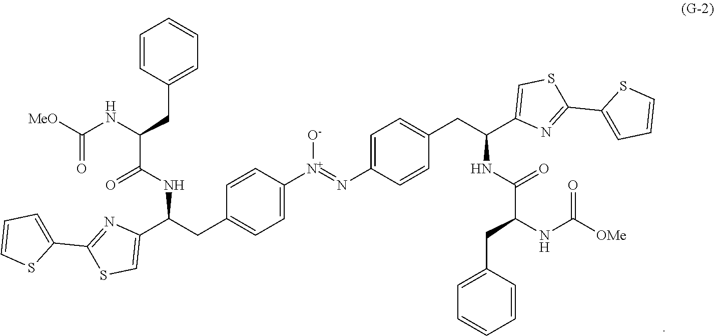 [SMILES: COC(=O)N[C@@H](Cc1ccccc1)C(=O)N[C@@H](Cc1ccc(/N=[N+](\[O-])c2ccc(C[C@H](NC(=O)[C@H](Cc3ccccc3)NC(=O)OC)c3csc(-c4cccs4)n3)cc2)cc1)c1csc(-c2cccs2)n1]
